SEQUENCE LISTING (18 sequences)

SEQ ID NO: 1 - protein, 165 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYENNPKGRSSKYGLLHCGNTEKDGKSPLTAHDARHTKAICL

SEQ ID NO: 2 - protein, 204 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEYIEEKKRVDICRETPNICPHSGENTEYDTIPHTNRTILKEDPANTVYSTVEIPKKMENPHSLLTMPDTPRLFAYENVI

SEQ ID NO: 3 - protein, 227 aa, Homo sapiens
MAGSPTCLTLIYILWQLTEHLSKPKVTMGLQSNKNGTCVTNLTCCMEHGEEDVIYTWKALGAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 4 - protein, 188 aa, Homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEEYIEEKKRVDICRETPNICPHSGENTEYDTIPHTNRTILKEDPANTVYSTVEIPKKMENPHSLLTMPDTPRLFAYENVI

SEQ ID NO: 5 - protein, 296 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEENNPKGRSSKYGLLHCGNTEKDGKSPLTAHDARHTKAICL

SEQ ID NO: 6 - protein, 149 aa, Homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEENNPKGRSSKYGLLHCGNTEKDGKSPLTAHDARHTKAICL

SEQ ID NO: 7 - protein, 241 aa, Homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEGDCLSPLHRRLCPGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 8 - protein, 201 aa, Homo sapiens
MGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 9 - protein, 189 aa, Homo sapiens
MAGSPTCLTLIYILWQLTEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEENNPKGRSSKYGLLHCGNTEKDGKSPLTAHDARHTKAICL

SEQ ID NO: 10 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 11 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 12 - DNA, 20 bp, Artificial Sequence
  Description of Artificial Sequence Synthetic primer
agggcgccagggggaagacg

SEQ ID NO: 13 - DNA, 20 bp, Artificial Sequence
  Description of Artificial Sequence Synthetic primer
gtctgccacaagatcatcca

SEQ ID NO: 14 - DNA, 19 bp, Artificial Sequence
  Description of Artificial Sequence Synthetic primer
tcgtagctgtgcaggaagc

SEQ ID NO: 15 - DNA, 20 bp, Artificial Sequence
  Description of Artificial Sequence Synthetic primer
cagaagctggaggaccagac

SEQ ID NO: 16 - DNA, 20 bp, Artificial Sequence
  Description of Artificial Sequence Synthetic primer
gtcggagaatgggttcagtt

SEQ ID NO: 17 - DNA, 20 bp, Artificial Sequence
  Description of Artificial Sequence Synthetic primer
atgttcgtcatgggtgtgaa

SEQ ID NO: 18 - DNA, 20 bp, Artificial Sequence
  Description of Artificial Sequence Synthetic primer
gtcttctgggtggcagtgat

(19) United States
(12) Patent Application Publication
(10) Pub. No.: US 2016/0159918 A1
(43) Pub. Date: Jun. 9, 2016

(54) METHODS FOR DIAGNOSING AND TREATING IMMUNE DISEASE

(71) Applicant: THE GENERAL HOSPITAL CORPORATION, Boston, MA (US)

(72) Inventors: Shiv PILLAI, Lexington, MA (US); John H. STONE, Sudbury, MA (US); Hamid MATTOO, Arlington, MA (US); Vinay S. MAHAJAN, Winthrop, MA (US)

(21) Appl. No.: 14/906,659

(22) PCT Filed: Jul. 24, 2014
(86) PCT No.: PCT/US14/48006

§ 371 Date: Jan. 21, 2016

Related U.S. Application Data
(60) Provisional application No. 61/857,807, filed on Jul. 24, 2013.

(51) Int. Cl. C07K 16/28; G01N 33/564

(52) U.S. Cl. CPC C07K 16/2896; G01N 33/564; G01N 2333/70596; G01N 2333/70546; G01N 2333/96436; G01N 2333/4703; G01N 2800/7095; G01N 2800/7052

(57) ABSTRACT

Provided are methods and assays relating to the treatment of an immune disease or disorder by administering an inhibitor that binds SLAMF7. Also provided are methods and assays related to the diagnosis of an immune disease or disorder by measuring expression level of SLAMF7 in a biological sample obtained from a subject.

### CROSS-REFERENCE TO RELATED APPLICATIONS

This application claims benefit under 35 U.S.C. §119(e) of U.S. Provisional Application Ser. No. 61/857,807 filed on Jul. 24, 2013, the contents of which are herein incorporated by reference in its entirety.

### TECHNICAL FIELD

The technical field relates to the diagnosis and treatment of immune diseases.

### GOVERNMENT SUPPORT

This invention was made with government support under Grant No. AI64930 awarded by the National Institutes of Health (NIH). The government has certain rights in the invention.

### BACKGROUND

IgG4-related disease (IgG4-RD) is a multi-organ chronic inflammatory condition characterized by tumefactive lesions, storiform fibrosis, and mild to moderate tissue eosinophilia (Stone JH, Zen Y, Deshpande V. IgG4-related disease. N Engl J Med 2012;366:539-51). The majority of patients also have substantial elevations in serum IgG4 concentrations. IgG4-RD includes subjects previously diagnosed with other disorders that were typically defined by the dominant pattern of organ involvement. Examples of such diagnoses that are now classified as part of the IgG4-RD spectrum are type I autoimmune pancreatitis, Mikulicz's syndrome, Reidel's thyroiditis, retroperitoneal fibrosis, Küttner's tumor, and sclerosing cholangitis, among others. The clinical manifestations of this syndrome have been reviewed elsewhere (Hamano H, Kawa S, Horiuchi A, et al. High serum IgG4 concentrations in patients with sclerosing pancreatitis. N Engl J Med 2001;344:732-8). Little is known about the pathogenesis of IgG4-RD, but autoantibodies have been identified in subsets of subjects (Kamisawa T, Egawa N, Nakajima H. Autoimmune pancreatitis is a systemic autoimmune disease. Am J Gastroenterol 2003;98:2811-2; Stone JH, Khosroshahi A, Deshpande V, et al. Recommendations for the nomenclature of IgG4-related disease and its individual organ system manifestations. Arthritis Rheum 2012;64:3061-7; Umehara H, Okazaki K, Masaki Y, et al. A novel clinical entity, IgG4-related disease (IgG4RD): general concept and details. Mod Rheumatol 2012;22:1-14) and depletion of B cells with rituximab results in striking clinical improvement (Khosroshahi A, Stone JH. A clinical overview of IgG4-related systemic disease. Curr Opin Rheumatol 2011;23:57-66).

Cytokines such as IL-4 and IL-10 have been reported within tissue lesions (Aoki S, Nakazawa T, Ohara H, et al Immunohistochemical study of autoimmune pancreatitis using anti-IgG4 antibody and patients' sera. Histopathology 2005;47:147-58; Nishimori I, Miyaji E, Morimoto K, Nagao K, Kamada M, Onishi S. Serum antibodies to carbonic anhydrase IV in patients with autoimmune pancreatitis. Gut 2005;54:274-81) and it is likely that CD4+ T cells play a role in disease pathogenesis (Okazaki K, Uchida K, Ohana M, et al. Autoimmune-related pancreatitis is associated with autoantibodies and a Th1/Th2-type cellular immune response. Gastroenterology 2000;118:573-81). However, these cytokines can be secreted by a number of cell types other than CD4+ T cells, including innate immune cells, innate lymphoid cells and B cells, and direct evidence implicating CD4+ T cells is lacking.

### SUMMARY

The methods and assays described herein are based, in part, on the discovery of the SLAMF7 receptor on the surface of T-cells in subjects having an immune disease (e.g., IgG4-RD or other fibrotic or inflammatory diseases) but not on the surface of T-cells of healthy subjects. We report herein that CD4⁺ T cells with a cytotoxic T lymphoid phenotype are clonally expanded in IgG4-RD subjects. These unusual CD4⁺ T cells express SLAMF7 and can synthesize and secrete IL-1β following TCR or TLR triggering and, apart from being expanded in the blood, are also found within diseased tissue sites. Their numbers decline concomitant with a clinical response to rituximab therapy, indicating a contributory role for these SLAMF7 expressing, IL-1β secreting CD4⁺ cytotoxic T cells in the pathogenesis of this systemic fibrotic disease. Thus, provided herein are methods and assays for diagnosing and/or treating an immune disease or disorder using an inhibitor that binds to SLAMF7 and/or IL-1β.

From a therapeutic standpoint, depleting SLAMF7-expressing cells, as well as, or, in some embodiments of the aspects described herein, inhibiting or neutralizing IL1β, represent novel, rational strategies for a range of immune-mediated conditions associated with severe tissue damage and fibrosis. A few biologics targeting SLAMF7 or IL1-β are already in the market or under advanced stages of drug development, and can be used in the aspects and embodiments related to therapeutic treatment methods described herein. For example, a humanized monoclonal antibody directed against the human SLAMF7, elotuzumab, has shown promise in patients with advanced multiple myeloma and is being pursued in phase III clinical trials (48). Anakinra, a non-glycosylated recombinant form of the naturally occurring IL-1β receptor antagonist which blocks inflammasome dependent IL1-β signaling has been successfully used in type 2 diabetes, asbestosis, and other conditions (49). Canakinumab is a moncolonal antibody that binds to and antagonizes IL-1β and is being studied in a number of clinical trials (50).

In summary, the studies described herein indicate that CD4⁺ CTLs or cytotoxic CD4+ T cells with a unique, hitherto undescribed phenotype clonally expand in the circulation and tissue sites and can mediate the pathological changes seen in IgG4-RD. These cells make a unique combination of cytokines, some of which have been shown to contribute to fibrosis in animal models, and the numbers of these cells correlate with clinical disease activity. Furthermore, therapeutic improvement in IgG4-RD mediated by B cell depletion is linked to a specific reduction of these CD4⁺ CTLs and not of naive T cells, regulatory T cells or memory T follicular helper cells. Examining untreated active disease has allowed the identification and characterization of clonally expanded effector T cells linked to disease and to the observation of their attenuation by rituximab.

Accordingly, in one aspect, the methods and assays provided herein relate to a method for treating a subject having an immune disease or disorder, the method comprising: administering a therapeutically effective amount of an inhibitor that binds SLAMF7 to a subject having an immune disease or disorder, thereby treating the immune disease or disorder.

In one embodiment of this aspect and all other aspects described herein, the inhibitor that binds SLAMF7 comprises an antibody or an antigen-binding fragment thereof. In some such embodiments, the antibody or an antigen-binding fragment thereof comprises elotuzumab.

In some embodiments of this aspect and all other aspects described herein, the method further comprises administering a therapeutically effective amount of an IL-1β inhibitor.

In another embodiment of this aspect and all other aspects described herein, the immune disease or disorder comprises an IgG4-RD spectrum disorder, a fibrotic disease, or other chronic inflammatory disease.

In another embodiment of this aspect and all other aspects described herein, the method further comprises a step of diagnosing the subject as having the immune disease or disorder.

Also provided herein, in another aspect, is a method for diagnosing an immune disease or disorder, the method comprising: (a) measuring the amount of SLAMF7 in a biological sample obtained from a suspected of having an immune disease or disorder, and (b) comparing the amount of SLAMF7 with a reference value, and if the amount of SLAMF7 is increased relative to the reference value, identifying the subject as having the immune disease or disorder.

In one embodiment of this aspect and all other aspects described herein, the immune disease or disorder comprises an IgG4-RD spectrum disorder or a fibrotic disease.

In another embodiment of this aspect and all other aspects described herein, the step of measuring the amount of SLAMF7 comprises contacting the biological sample with an antibody specific for SLAMF7.

In another embodiment of this aspect and all other aspects described herein, the reference value is obtained from a subject or population of subjects lacking a detectable immune disease or disorder.

In another embodiment of this aspect and all other aspects described herein, the method further comprises measuring expression of at least one additional cytotoxic CD4+ T-cell marker.

In another embodiment of this aspect and all other aspects described herein, the at least one additional T-cell marker is selected from the group consisting of: CD4, CD11b, 2B4, granzyme, perform, and T-bet transcription factor.

In another aspect, provided herein is an assay comprising: (a) measuring the amount of SLAMF7 in a biological sample obtained from a subject having, or suspected of having, an immune disease or disorder, and (b) comparing the amount of SLAMF7 with a reference value, and if the amount of SLAMF7 is increased relative to the reference value, identifying the subject as having, or at risk of developing, an immune disease or disorder.

In one embodiment of this aspect and all other aspects described herein, the immune disease or disorder comprises an IgG4-RD spectrum disorder, a fibrotic disorder or a chronic inflammatory disease.

In another embodiment of this aspect and all other aspects described herein, the step of measuring the amount of SLAMF7 comprises contacting the biological sample with an antibody specific for SLAMF7.

In another embodiment of this aspect and all other aspects described herein, the reference value is obtained from a subject or population of subjects lacking a detectable immune disease or disorder.

In another embodiment of this aspect and all other aspects described herein, the assay further comprises measuring expression of at least one additional cytotoxic CD4+ T-cell marker.

In another embodiment of this aspect and all other aspects described herein, the at least one additional cytotoxic CD4+ T-cell marker is selected from the group consisting of: CD4, CD11b, 2B4, granzyme, perform, and T-bet transcription factor.

Also provided herein, in some aspects are methods and uses for inhibiting or targeting cytotoxic CD4+ T cells in a subject in need thereof, the methods comprising administering to a subject an effective amount of a pharmaceutical composition comprising a SLAMF7 inhibitor, an IL-1β inhibitor, or a combination thereof.

In some embodiments of these methods and all such methods described herein, the SLAMF7 inhibitor binds SLAMF7.

In some embodiments of these methods and all such methods described herein, the SLAMF7 inhibitor reduces mRNA or protein expression of one or more SLAMF7 isoforms.

In some embodiments of these methods and all such methods described herein, the SLAMF7 inhibitor is an anti-SLAMF7 antibody or antigen-binding fragment thereof, a small molecule SLAMF7 inhibitor, an RNA or DNA aptamer that binds or physically interacts with one or more SLAMF7 isoforms, a SLAMF7 structural analog, a SLAMF7specific antisense molecule, or a SLAMF7 specific siRNA molecule. In some such embodiments, the SLAMF7 inhibitor is the humanized monoclonal antibody elotuzumab.

In some embodiments of these methods and all such methods described herein, the IL-1β inhibitor binds IL-1β.

In some embodiments of these methods and all such methods described herein, the IL-1β inhibitor reduces mRNA or protein expression of IL-1β.

In some embodiments of these methods and all such methods described herein, IL-1β inhibitor is an anti-IL-1β antibody or antigen-binding fragment thereof, a small molecule IL-1β inhibitor, an RNA or DNA aptamer that binds or physically interacts with IL-1β, an IL-1β structural analog, an IL-1β specific antisense molecule, or an IL-1β specific siRNA molecule. In some such embodiments, the IL-1β inhibitor is the monoclonal antibody canakinumab. In some such embodiments, the IL-1β inhibitor is a IL-1β receptor antagonist, such as, for example, anakinra.

In some embodiments of these methods and all such methods described herein, the subject being administered the SLAMF7 inhibitor, the IL-1β inhibitor, or the combination thereof, is diagnosed as having an immune disease or disorder. In some such embodiments, the immune disease or disorder comprises an IgG4-RD spectrum disorder, a fibrotic disorder or a chronic inflammatory disease.

In some embodiments of these methods and all such methods described herein, the subject being administered the SLAMF7 inhibitor, the IL-1β inhibitor, or the combination thereof has an immune disease or disorder that comprises a population of cytotoxic CD4+ T cells. In some embodiments of these methods, the population of cytotoxic CD4+ T cells expresses two or more cytotoxic CD4+ T cell markers. In some such embodiment, the two or more cytotoxic CD4+ T cell markers are selected from the group consisting of CD4, T-bet, SLAMF7, CD11b, 2B4, CD28, perform, granzyme, ThPOK, and Runx3.

In some embodiments of these methods and all such methods described herein, the method further comprises identifying a population of cytotoxic CD4+ T cells in the subject.

In some embodiments of these methods and all such methods described herein, the method comprises further administering an anti-CD20 monoclonal antibody, such as, for example, rituximab.

Also provided herein, in some aspects, are pharmaceutical compositions comprising a SLAMF7 inhibitor, an IL-1β inhibitor, or a combination thereof for use in inhibiting or targeting cytotoxic CD4+ T cells in a subject in need thereof.

In some embodiments of these uses and all such uses described herein, the SLAMF7 inhibitor binds SLAMF7.

In some embodiments of these uses and all such uses described herein, the SLAMF7 inhibitor reduces mRNA or protein expression of one or more SLAMF7 isoforms.

In some embodiments of these uses and all such uses described herein, SLAMF7 inhibitor is an anti-SLAMF7 antibody or antigen-binding fragment thereof, a small molecule SLAMF7 inhibitor, an RNA or DNA aptamer that binds or physically interacts with one or more SLAMF7 isoforms, a SLAMF7 structural analog, a SLAMF7specific antisense molecule, or a SLAMF7 specific siRNA molecule. In some such embodiments, the SLAMF7 inhibitor is the humanized monoclonal antibody elotuzumab.

In some embodiments of these uses and all such uses described herein, the IL-1β inhibitor binds IL-1β.

In some embodiments of these uses and all such uses described herein, the IL-1β inhibitor reduces mRNA or protein expression of IL-1β.

In some embodiments of these uses and all such uses described herein, the IL-1β inhibitor is an anti-IL-1β antibody or antigen-binding fragment thereof, a small molecule IL-1β inhibitor, an RNA or DNA aptamer that binds or physically interacts with IL-1β, an IL-1β structural analog, an IL-1β specific antisense molecule, or an IL-1β specific siRNA molecule. In some such embodiments, the IL-1β inhibitor is the monoclonal antibody canakinumab. In some such embodiments, the IL-1β inhibitor is an IL-1β receptor antagonist, such as, for example, anakinra.

In some embodiments of these uses and all such uses described herein, the subject in need thereof is diagnosed as having an immune disease or disorder. In some such embodiments, the immune disease or disorder comprises an IgG4-RD spectrum disorder, a fibrotic disorder or a chronic inflammatory disease.

In some embodiments of these uses and all such uses described herein, the subject in need thereof has an immune disease or disorder that comprises a population of cytotoxic CD4+ T cells. In some embodiments of these uses, the population of cytotoxic CD4+ T cells expresses two or more cytotoxic CD4+ T cell markers. In some such embodiment, the two or more cytotoxic CD4+ T cell markers are selected from the group consisting of CD4, T-bet, SLAMF7, CD11b, 2B4, CD28, perform, granzyme, ThPOK, and Runx3.

In some embodiments of these uses and all such uses described herein, the use comprises further administering an anti-CD20 monoclonal antibody, such as, for example, rituximab.

### BRIEF DESCRIPTION OF THE FIGURES

FIGS. 1A-1C show oligoclonal expansions of T_{EM} (T effector memory) cells in IgG4-RD. The gating scheme used for flow cytometry analysis of T_{EM} cells and an expansion of antigen-experienced CD4⁺CD45RA⁻CD45RO⁺T CD62^{lo}VD27^{lo}T_{EM} cells in an IgG4-RD subject is depicted in FIG. 1A. FIG. 1B. The T-cell receptor β-chain (TCRB) repertoire of sorted T_{EM} cells from three subjects with IgG4-RD is shown as 3D histograms. FIG. 1C. The dominant expanded T_{EM} clone (Vβ19⁺) identified by Next-Generation Sequencing and shown in panel B for index patient 414, is observed in the peripheral blood.

FIGS. 2A-2D show data indicating expanded T_{EM} cells show a distinct gene expression profile. Heat-map depicts a conserved pattern of differentially expressed immunology-related genes in T_{EM} cells from two of the originally analyzed IgG4-RD patients from FIG. 1 compared to CD4⁺CD45RO⁺ T cells from healthy controls (FIG. 2A). Key hits from the gene expression analysis were validated using flow cytometry on T_{EM} cells from subjects studied in FIG. 1 (FIG. 2B) and clonally expanded subsets of T cells were identified using TCR-Vβ specific antibodies (FIG. 2C). FIG. 2C depicts the expanded clone identified from the repertoire analysis of subject 414 shown in FIG. 1B. FIG. 2D is a micrograph comparing flow cytometry data of T-bet vs. SLAMF7 in a healthy control and in an index subject with IgG4-RD showing SLAMF7 expression in the expanded T_{EM} cells.

FIG. 3 is a micrograph comparing flow cytometry data of T-bet vs. SLAMF7 in a healthy control and in a subject having granulomatosis with polyangiitis (also known as Wegener's disease) showing SLAMF7 on T_{EM} cells in granulomatosis with polyangiitis (formerly Wegener's disease).

FIGS. 4A-4D demonstrate expansions of T_{EM} cells in IgG4-RD. FIG. 4A shows the frequency of GATA-3⁺ Th2 cells in atopic and non-atopic subsets of IgG4-RD subjects compared to healthy controls (mean±s.d., p-values for unpaired t-tests are shown) FIG. 4B shows the gating scheme used for flow cytometry analysis of CD4⁺CD45RO⁺ antigen-experienced cells and CD4⁺CD62L^{lo}CD27^{lo} T_{EM} cells is depicted in a representative IgG4-RD patient (P21) and a healthy control subject. FIGS. 4C-4D shows the numbers of CD4⁺CD45RO⁺CD62L^{lo}CD27^{lo} TEM cells (4C), and CD4⁺CD45RO⁺CD39⁺CD25⁺Foxp3⁺ T_{reg} cells (4D) in peripheral blood of IgG4-RD subjects (mean±s.d., number of subjects tested and p-values for unpaired t-tests are shown).

FIGS. 5A-5D show TCR Vβ repertoire of expanded T_{EM} cells. FIG. 5A shows TCR Vβ repertoire of the expanded circulating T_{EM} subset in a IgG4-RD subjects represented as bubble charts, where the size and color corresponds to the frequency of the observed Vβ-Jβ rearrangements. FIG. 5B shows cumulative distribution of clone frequencies in CD4⁺ T_{EM} cells from 5 IgG4-RD subjects. The minimum number of clones accounting for 10% (D10) and 50% (D50) of the clonal diversity are shown. FIG. 5C shows the dominant expanded _{TEM} clone (Vβ19⁺) identified by Next-Generation Sequencing in FIG. 5A detected by flow cytometry in the peripheral blood. FIG. 5D shows dominant clones in subjects P21, P23 and P27 as detected using Vβ-specific antibodies compared with background frequencies in controls and non-T_{EM} cells in respective patients.

FIGS. 6A-6G demonstrate that expanded T_{EM} cells in IgG4-RD subjects show a distinct gene expression profile. FIG. 6A shows a heat map depicting differentially expressed immune-related genes in T_{EM} cells from four patients compared to CD4⁺CD45RO⁺ T cells from four healthy controls. FIGS. 6B-6C show key hits from the gene expression analysis from (6A) validated using flow cytometry in T_{EM} cells (6B) as well as in expanded clones of T cells identified using TCR Vβ-specific antibodies (6C). FIG. 5D shows the levels of ThPOK and Runx3 and were quantified by qRT-PCR in CD4+SLAMF7+ CTLs from IgG4-RD subjects compared to CD4⁺CD45RO⁺ T cells healthy controls. Error bars show SEM. FIG. 5E shows gating strategy to depict the CD8α expression on CD4⁺T-bet⁺ CTLs from a representative patient (P21). FIG. 5F shows Granzyme B and CD107a staining on CD4⁺ CTLs from an IgG4-RD patient, before and after stimulation with anti-CD3 (3 μg/ml). FIG. 5G shows the cytotoxicity of in vitro expanded CD4+ CTLs derived from two subjects (P1 and P21) against an allogeneic EBV-transformed B cell target cell line was measured after 12 hours of co-culture with or without anti-CD3 (10 μg/mL) using Annexin V staining at varying CD4⁺ CTL: target ratios.

FIGS. 7A-7E demonstrate expansion of CD4⁺SLAMF7⁺ CTLs in IgG4-RD subjects. FIGS. 7A-7B show expansion of CD4⁺SLAMF7⁺ CTLs in IgG4-RD subjects (mean±s.d.) (7A) and in atopic and non-atopic subsets of IgG4-RD subjects (7B). FIG. 7C shows immunofluorescence staining of CD4⁺SLAMF7⁺ CTLs in the affected tissues of IgG4-RD subjects (submandibular salivary gland biopsy from P25, nasal palate biopsy from P31 and retroperitoneal biopsy from P40). CD4, DAPI and SLAMF7 staining are shown. FIGS. 7D-7E show CD4⁺CD62L^{lo}CD27^{lo}SLAMF7⁺ CTLs in the aortic wall of a subject with IgG4-related aortitis (7D) and the involved nasal septum of a subject with IgG4-RD (7E).

FIGS. 8A-8C demonstrate that Th2 cell expansions in IgG4-RD are polyclonal. FIG. 8A shows TCR Vβ repertoire of the expanded circulating CD4⁺SLAMF7⁺ CTLs in an IgG4-RD subject (P8), contrasted with the expanded Th2 subset from the same individual. The repertoire is represented as bubble charts, where the size and color corresponds to the frequency of the observed Vβ-Jβ rearrangements. FIG. 8B shows cumulative distribution of clone frequencies in CD4⁺SLAMF7⁺ CTLs and CD4⁺GATA-3⁺ cells. FIG. 8C shows intracellular staining for IFN-γ and IL-4 in CD4⁺ CTLs identified using T-bet staining from seven IgG4-RD patients after restimulation with PMA (100 ng/mL) and ionomycin (100 ng/mL). The filled histogram depicts an unstimulated control.

FIGS. 9A-9D demonstrate that expanded CD4⁺ CTLs from IgG4-RD patients secrete IL1-β. FIGS. 9A-9B show ELISPOT detection of the frequency of IL1-13 producers among re-stimulated CD4⁺CD45RO⁺ T cells from seven IgG4-RD subjects compared to healthy donors (error bars show SEM, unpaired t-test) (9A) and CD4⁺ CTLs from four IgG4-RD subjects compared to CD4⁺CD45RA⁺ T cells from healthy controls (p<0.05, paired t-test) (9B). FIG. 9C shows western Blot detection of IL1-β from culture supernatants of in vitro expanded T cells, maintained in IL-2 for two weeks (10 ng/mL). Supernatants from CD4⁺CD45RO⁺ T cells from a healthy donor and CD4⁺CD45RO⁺SLAMF7⁺ or CD4⁺CD45RO⁺SLAMF7 T cells from an IgG4-RD subject were used without any stimulation (US), with 5 μg/mL LPS or 3 μg/mL anti-CD3. LPS stimulated PBMCs from a healthy donor were used as a positive control. FIG. 9D shows IL-1β-producing CD4⁺ cells in the tissues of IgG4-RD subjects (Lacrimal gland biopsy from P3 and submandibular gland biopsy from P25). CD4, DAPI and IL-1β staining are shown.

FIGS. 10A-10D show Rituximab-mediated depletion of CD4⁺ CTLs. FIG. 10A shows change in the number of CD4⁺ SLAMF7⁺ CTLs (n=8 subjects) followed for 6-14 months after rituximab infusion. FIG. 10B shows effect of rituximab on CD4⁺ CD45RA⁺ naïve T cell counts in the peripheral blood of IgG4-RD Patients. FIG. 10C shows decline in the number and proportion of the expanded CD4⁺ CTL clone tracked using a TCR-Vβ specific antibody following rituximab therapy in an IgG4-RD subject (P21). FIG. 10D shows decline in circulating CD4⁺ CTL number at day 70-95 following rituximab therapy (normalized to the pretreatment levels) is plotted against the IgG4-RD Responder Index, a clinical measure of disease activity.

FIGS. 11A-11B demonstrate that CD4⁺ SLAMF7⁺ CTLs are expanded in IgG4-RD and other inflammatory fibrotic diseases. FIG. 11A shows circulating CD4⁺SLAMF7⁺ counts in healthy controls, IgG4-RD subjects and in subjects with other immune-mediated fibrotic diseases (sarcoidosis, scleroderma, rheumatoid arthritis Wegener's granulomatosis) plotted both as a group and individually. Error bars show SEM, Wilcoxon rank sum test, p<0.01. FIG. 11B shows a model of inflammatory fibrosis driven by CD4⁺CTLs contrasted with the Th2 cell-mediated fibrosis seen in allergic disorders and helminthic infestations.

FIG. 12 demonstrates increased frequency of CD4⁺CD45RO⁺ cells in peripheral blood of IgG4-RD patients. Frequency of total CD4⁺CD45RO⁺ T effector/memory cells in IgG4-RD subjects (n=19) compared to healthy controls (n=14). Data are plotted as mean±standard deviation, p values are shown.

FIG. 13 shows in vitro culture of CD4⁺ CTLs from IgG4-RD subjects. Flow-sorted CD4⁺SLAMF7⁺ CTLs from an IgG4-RD patient were stimulated with anti-human CD3 (3 μg/mL)+anti-human CD28 (1 μg/mL) in presence of recombinant human IL-2 (20 ng/mL) and their phenotype was checked after 2 weeks of culture.

FIG. 14 demonstrates cytotoxicity of CD4⁺ CTLs from IgG4-RD subjects. The cytotoxicity of CD4⁺ CTLs from two patients against allogeneic EBV-transformed B cell target was measured 12 hours of co-culture with or without anti-CD3 (10 μg/mL) using DAPI and Annexin V staining at varying CD4+ CTL to target ratios.

FIGS. 15A-15B demonstrates cytotoxicity of CD4⁺ CTLs from IgG4-RD subjects. Immunofluorescence staining of CD4⁺SLAMF7⁺ CTLs in the affected tissues of IgG4-RD subjects (Lacrimal gland biopsy from P3, Lymph node biopsy from P11, Laryngeal biopsy from P27, and nasal septum biopsy from P43). CD4, DAPI and SLAMF7 staining are shown.

FIG. 16 demonstrates expanded CD4⁺ CTLs clones from IgG4-RD patients secrete IFN-γ. Intracellular staining for IFN-γ and IL-4 in expanded clones of CD4⁺ CTLs identified using Vβ and T-bet staining from two IgG4-RD patients after restimulation with PMA (100 ng/mL) and ionomycin (100 ng/mL).

FIG. 17 demonstrates effect of rituximab on CD4⁺ T cell subsets. Effect of rituximab on CD4⁺CXCR5⁺ T_{FH} cells, CD4⁺CD25⁺Foxp3⁺ T_{reg} cells and CD4⁺GATA-3⁺ Th2 cell counts in the peripheral blood of IgG4-RD patients 90-120 days after rituximab therapy.

### DETAILED DESCRIPTION

Methods and assays are provided herein that relate to a method for treating a subject having an immune disease or disorder, the methods comprising: administering a therapeutically effective amount of an inhibitor that binds SLAMF7, an inhibitor that binds IL1-β, or an inhibitor that binds SLAMF7 and an inhibitor that binds IL1-β, to a subject having an immune disease or disorder, thereby treating the immune disease or disorder. In addition, methods and assays are provided herein that relate to a method of diagnosing an immune disease or disorder, for example, by measuring the level of expression of SLAMF7 in a biological sample obtained from a subject.

The studies described herein indicate that CD4+ CTLs or cytotoxic CD4+ T cells with a unique, hitherto undescribed phenotype clonally expand in the circulation and tissue sites and can mediate the pathological changes seen in IgG4-RD. These cells make a unique combination of cytokines, some of which have been shown to contribute to fibrosis in animal models, and the numbers of these cells correlate with clinical disease activity. Furthermore, therapeutic improvement in IgG4-RD mediated by B cell depletion is linked to a specific reduction of these CD4+ CTLs and not of naive T cells, regulatory T cells or memory T follicular helper cells. Examining untreated active disease has allowed the identification and characterization of clonally expanded effector T cells linked to disease and to the observation of their attenuation by rituximab. Accordingly, as demonstrated herein, depleting SLAMF7-expressing cells, as well as/or, in some embodiments of the aspects described herein, inhibiting or neutralizing IL1-β, represent novel, rational strategies for a range of immune-mediated conditions associated with severe tissue damage and fibrosis.

### Definitions

As used herein the term “IgG4-related disease (IgG4-RD)” refers to a fibroinflammatory condition characterized, in part, by tumefactive lesions and storiform fibrosis (see e.g., Stone, JH. et al. Arthritis and Rheumatism (2012) 64(10):3061-3067, which is herein incorporated by reference in its entirety). Exemplary disorders that fall within the IgG4-RD spectrum of disorders include, but are not limited to, type I autoimmune pancreatitis, Mikulicz's syndrome, Reidel's thyroiditis, retroperitoneal fibrosis, Küttner's tumor, sclerosing cholangitis, eosinophilic angiocentric fibrosis, multifocal fibrosclerosis, lymphoplasmacytic sclerosing pancreatitis, autoimmune pancreatitis, inflammatory pseudotumor, fibrosing medistinitis, sclerosing mesenteritis, retroperitoneal fibrosis (Ormond disease), periarteritis/periortitis, inflammatory aortic aneurysm, cutaneous pseudolymphoma, idiopathic hypertrophic pachymeningitis, idiopathic tubulointerstitial nephritis, idiopathic hypocomplementemic tubulointerstitial nephritis with extensive tubulointerstitial deposits, and idiopathic cervical fibrosis.

As used herein, the term “fibrotic disease” refers to a condition that is associated with the formation of excess or aberrant fibrous connective tissue in an organ or tissue. Such fibrous connective tissue can be due to a reparative or reactive process and can affect nearly all tissues and organ systems. Exemplary fibrotic diseases can include, but are not limited to, interstitial lung disease(s), liver cirrhosis, liver fibrosis resulting from chronic hepatitis B or C infection, kidney disease, heart or cardiovascular diseases (such as, for example, coronary artery disease, cardiomyopathy, hypertensive heart disease, cor pulmonale, cardiac dysrhythmias, inflammatory heart disease, endocarditis, inflammatory cardiomegaly, myocarditis, valvular heart disease, cerebrovascular disease, peripheral arterial disease, and rheumatic heart disease); systemic sclerosis (e.g., both diffuse and limited variants), systemic scleroderma, local scleroderma (e.g., morphea), keloids and hypertrophic scars, atherosclerosis, restenosis, eye diseases including macular degeneration and retinal and vitreal retinopathy, excessive scarring resulting from surgery, chemotherapeutic drug-induced fibrosis, radiation-induced fibrosis, injuries, burns, inappropriate fibrotic tissue remodeling, cancer metastasis, chronic graft rejection in transplant recipients, chronic lung disorders, including asthma, pneumoconioses, lung infections, fibrotic lung diseases such as fibrotic interstitial lung diseases, among which are included usual interstitial pneumonia (UIP), and the fibrotic variant of non-specific interstitial pneumonia (NSIP), among others.

Other immune diseases and disorders for use with the methods and assays described herein include, but are not limited to, Acquired Immunodeficiency Disease Syndrome; Acquired Immunodeficiency Related Diseases; acquired pernicious anaemia; acute coronary syndromes; acute and chronic pain (different forms of pain); acute idiopathic polyneuritis; acute immune disease associated with organ transplantation; acute or chronic immune disease associated with organ transplantation; acute inflammatory demyelinating polyradiculoneuropathy; acute ischemia; acute liver disease; acute rheumatic fever; acute transverse myelitis; Addison's disease; adult (acute) respiratory distress syndrome; adult Still's disease; alcoholic cirrhosis; alcohol-induced liver injury; allergic diseases; allergy; alopecia; alopecia areata; Alzheimer's disease; anaphylaxis; ankylosing spondylitis; ankylosing spondylitis associated lung disease; anti-phospholipid antibody syndrome; aplastic anemia; arteriosclerosis; arthropathy; asthma; atheromatous disease/arteriosclerosis; atherosclerosis; atopic allergy; atopic eczema; atopic dermatitis; atrophic autoimmune hypothyroidism; autoimmune bullous disease; autoimmune dermatitis; autoimmune diabetes; autoimmune disorder associated with streptococcus infection; autoimmune enteropathy; autoimmune haemolytic anaemia; autoimmune hepatitis; autoimmune hearing loss; autoimmune lymphoproliferative syndrome (ALPS); autoimmune mediated hypoglycaemia; autoimmune myocarditis; autoimmune neutropenia; autoimmune premature ovarian failure; autoimmune thrombocytopenia (AITP); autoimmune thyroid disease; autoimmune uveitis; bronchiolitis obliterans; Behget's disease; blepharitis; bronchiectasis; bullous pemphigoid; cachexia; cardiovascular disease; catastrophic antiphospholipid syndrome; celiac disease; cervical spondylosis; chlamydia; choleosatatis; chronic active hepatitis; chronic eosinophilic pneumonia; chronic fatigue syndrome; chronic immune disease associated with organ transplantation; chronic ischemia; chronic liver diseases; chronic mucocutaneous candidiasis; cicatricial pemphigoid; clinically isolated syndrome (CIS) with risk for multiple sclerosis; common varied immunodeficiency (common variable hypogammaglobulinaemia); connective tissue disease associated interstitial lung disease; conjunctivitis; Coombs positive haemolytic anaemia; childhood onset psychiatric disorder; chronic obstructive pulmonary disease (COPD); Crohn's disease; cryptogenic autoimmune hepatitis; cryptogenic fibrosing alveolitis; dacryocystitis; depression; dermatitis scleroderma; dermatomyositis; dermatomyositis/polymyositis associated lung disease; diabetic retinopathy; diabetes mellitus; dilated cardiomyopathy; discoid lupus erythematosus; disk herniation; disk prolapse; disseminated intravascular coagulation; drug-induced hepatitis; drug-induced interstitial lung disease; drug induced immune hemolytic anemia; endocarditis; endometriosis; endophthalmitis; enteropathic synovitis; episcleritis; erythema multiforme; erythema multiforme major; female infertility; fibrosis; fibrotic lung disease; gestational pemphigoid; giant cell arteritis (GCA); glomerulonephritides; goitrous autoimmune hypothyroidism (Hashimoto's disease); Goodpasture's syndrome; gouty arthritis; graft versus host disease (GVHD); Grave's disease; group B streptococci (BGS) infection; Guillain-Barré syndrome (BGS); haemosiderosis associated lung disease; hay fever; heart failure; hemolytic anemia; Henoch-Schoenlein purpura; hepatitis B; hepatitis C; Hughes syndrome; Huntington's chorea; hyperthyroidism; hypoparathyroidism; idiopathic leucopaenia; idiopathic thrombocytopaenia; idiopathic Parkinson's disease; idiopathic interstitial pneumonia; idiosyncratic liver disease; IgE-mediated allergy; immune hemolytic anemia; inclusion body myositis; infectious diseases; infectious ocular inflammatory disease; inflammatory bowel disease; inflammatory demyelinating disease; inflammatory heart disease; inflammatory kidney disease; insulin dependent diabetes mellitus; interstitial pneumonitis; IPF/UIP; iritis; juvenile chronic arthritis; juvenile pernicious anaemia; juvenile rheumatoid arthritis (JRA); Kawasaki's disease; keratitis; keratojunctivitis sicca; Kussmaul disease or Kussmaul-Meier disease; Landry's paralysis; Langerhan's cell histiocytosis; linear IgA disease; livedo reticularis; Lyme arthritis; lymphocytic infiltrative lung disease; macular degeneration; male infertility idiopathic or NOS; malignancies; microscopic vasculitis of the kidneys; microscopic polyangiitis; mixed connective tissue disease associated lung disease; Morbus Bechterev; motor neuron disorders; mucous membrane pemphigoid; multiple sclerosis (all subtypes: primary progressive, secondary progressive, relapsing remitting etc.); multiple organ failure; myalgic encephalitis/royal free disease; myasthenia gravis; myelodysplastic syndrome; myocardial infarction; myocarditis; nephrotic syndrome; nerve root disorders; neuropathy; non-alcoholic steatohepatitis; non A non B hepatitis; optic neuritis; organ transplant rejection; osteoarthritis; osteolysis; ovarian cancer; ovarian failure; pancreatitis; parasitic diseases; Parkinson's disease; pauciarticular JRA; pemphigoid; pemphigus foliaceus; pemphigus vulgaris; peripheral artery occlusive disease (PAOD); peripheral vascular disease (PVD); peripheral artery disease (PAD); phacogenic uveitis; phlebitis; polyarteritis nodosa (or periarteritis nodosa); polychondritis; polymyalgia rheumatica; poliosis; polyarticular JRA; polyendocrine deficiency syndrome; polymyositis; polyglandular deficiency type I and polyglandular deficiency type II; polymyalgia rheumatica (PMR); postinfectious interstitial lung disease; post-inflammatory interstitial lung disease; post-pump syndrome; premature ovarian failure; primary biliary cirrhosis; primary myxoedema; primary Parkinsonism; primary sclerosing cholangitis; primary sclerosing hepatitis; primary vasculitis; prostate and rectal cancer and hematopoietic malignancies (leukemia and lymphoma); prostatitis; psoriasis; psoriasis type 1; psoriasis type 2; psoriatic arthritis; psoriatic arthropathy; pulmonary hypertension secondary to connective tissue disease; pulmonary manifestation of polyarteritis nodosa; pure red cell aplasia; primary adrenal insufficiency; radiation fibrosis; reactive arthritis; Reiter's disease; recurrent neuromyelitis optica; renal disease NOS; restenosis; rheumatoid arthritis; rheumatoid arthritis associated interstitial lung disease; rheumatic heart disease; SAPHO (synovitis, acne, pustulosis, hyperostosis, and osteitis); sarcoidosis; schizophrenia; Schmidt's syndrome; scleroderma; secondary amyloidosis; shock lung; scleritis; sciatica; secondary adrenal insufficiency; sepsis syndrome; septic arthritis; septic shock; seronegative arthropathy; silicone associated connective tissue disease; Sjogren's disease associated lung disease; Sjorgren's syndrome; Sneddon-Wilkinson dermatosis; sperm autoimmunity; spondyloarthropathy; spondylitis ankylosans; Stevens-Johnson syndrome (SJS); Still's disease; stroke; sympathetic ophthalmia; systemic inflammatory response syndrome; systemic lupus erythematosus; systemic lupus erythematosus associated lung disease; systemic sclerosis; systemic sclerosis associated interstitial lung disease; Takayasu's disease/arteritis; temporal arteritis; Th2 Type and Th1 Type mediated diseases; thyroiditis; toxic shock syndrome; toxoplasmic retinitis; toxic epidermal necrolysis; transverse myelitis; TRAPS (Tumor-necrosis factor receptor type 1 (TNFR)-Associated Periodic Syndrome); type B insulin resistance with acanthosis nigricans; type 1 allergic reaction; type-1 autoimmune hepatitis (classical autoimmune or lupoid hepatitis); type-2 autoimmune hepatitis (anti-LKM antibody hepatitis); type II diabetes; ulcerative colitic arthropathy; ulcerative colitis; urticaria; usual interstitial pneumonia (UIP); uveitis; vasculitic diffuse lung disease; vasculitis; vernal conjunctivitis; viral retinitis; vitiligo; Vogt-Koyanagi-Harada syndrome (VKH syndrome); Wegener's granulomatosis; wet macular degeneration; wound healing; _{Y}ersinia and salmonella associated arthropathy.

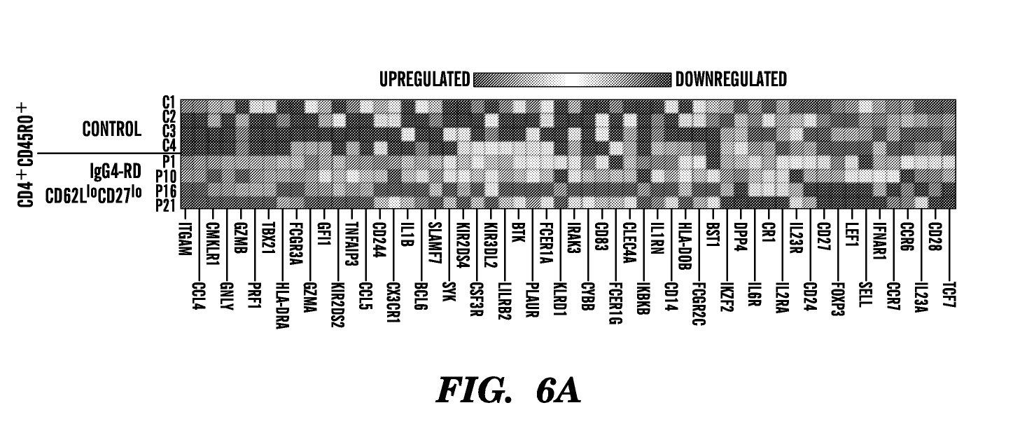

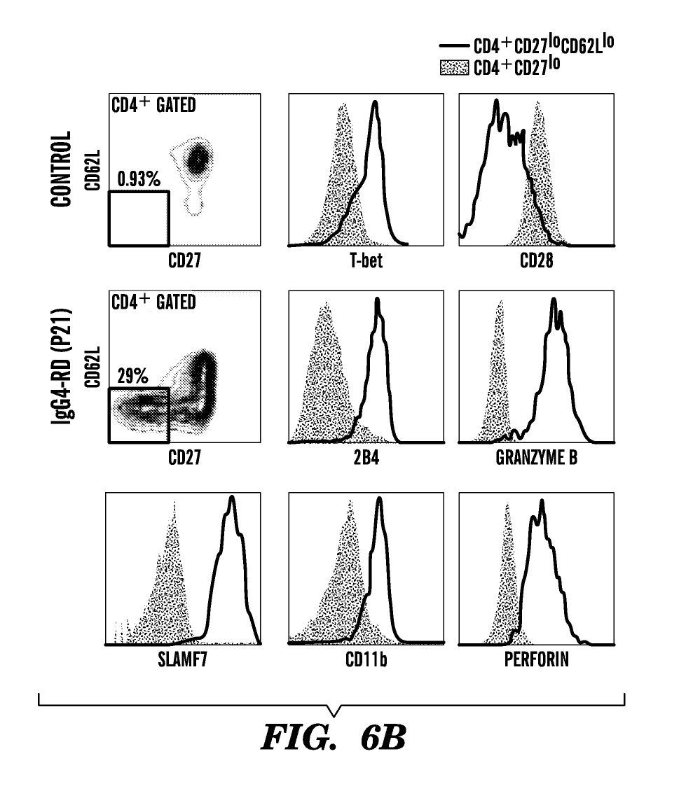

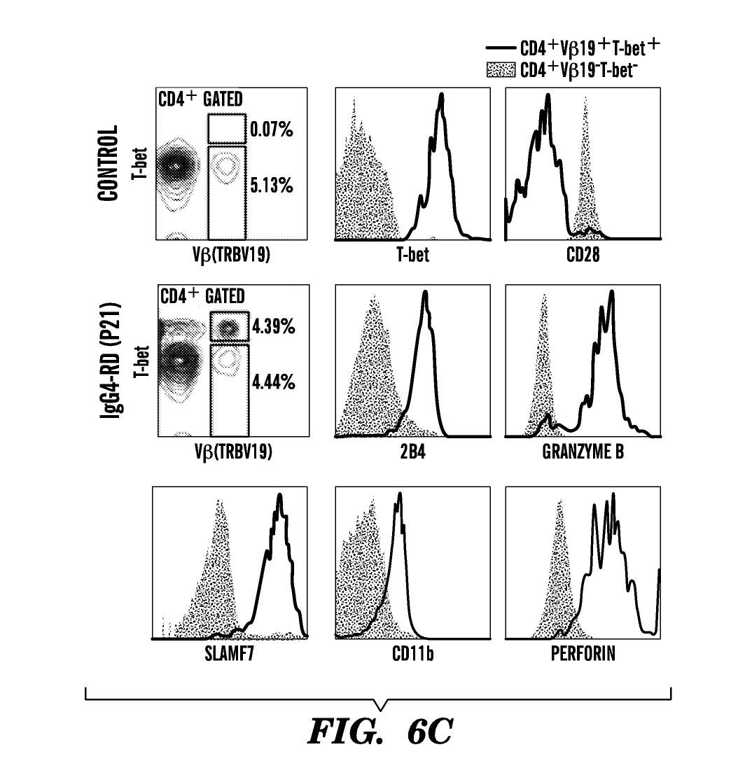

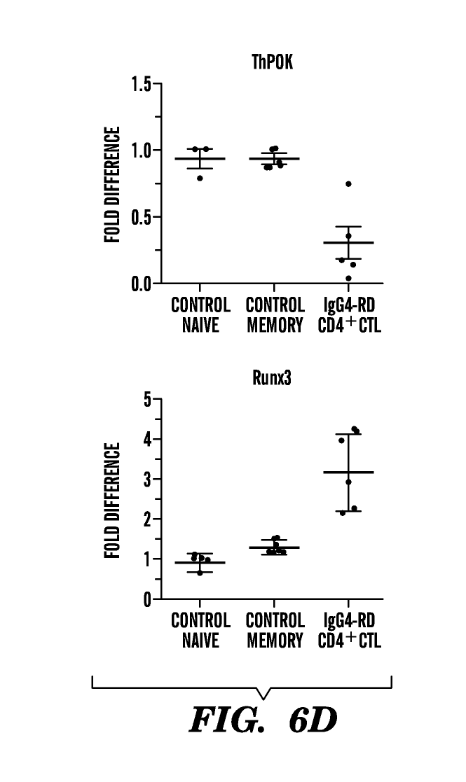

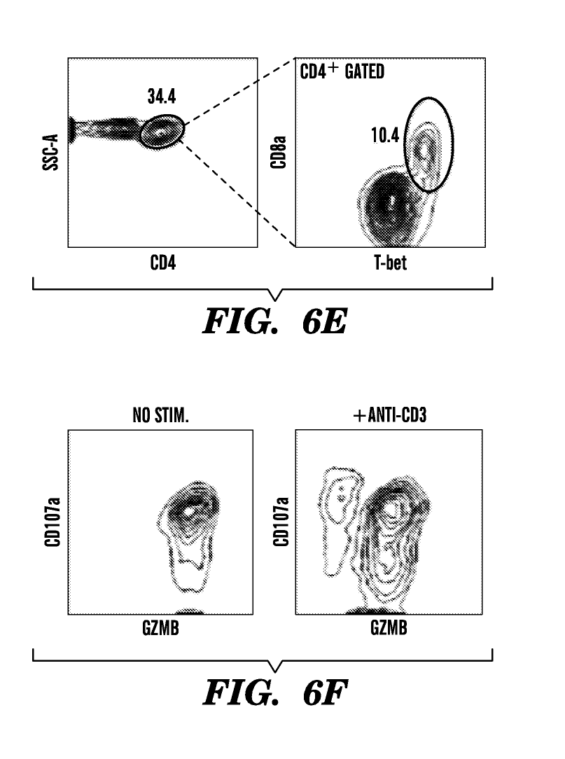

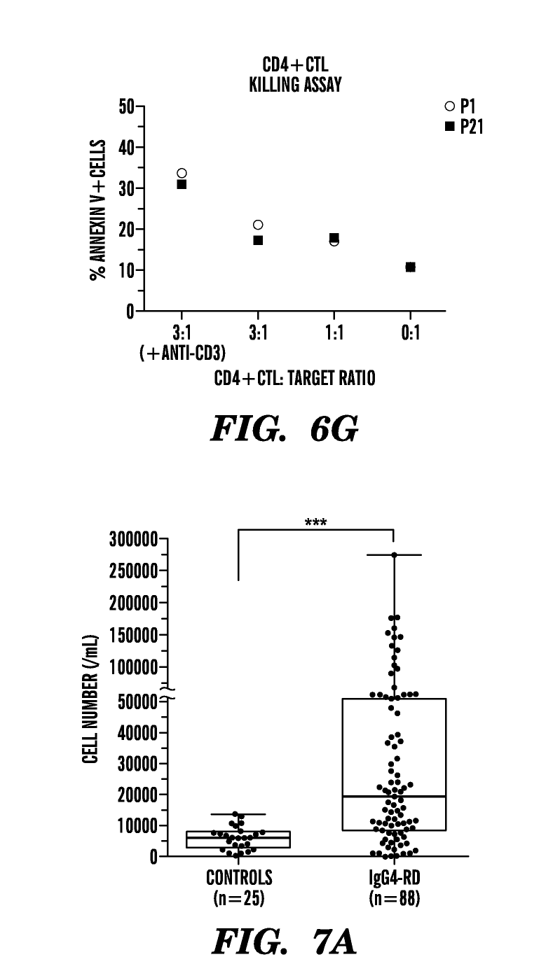

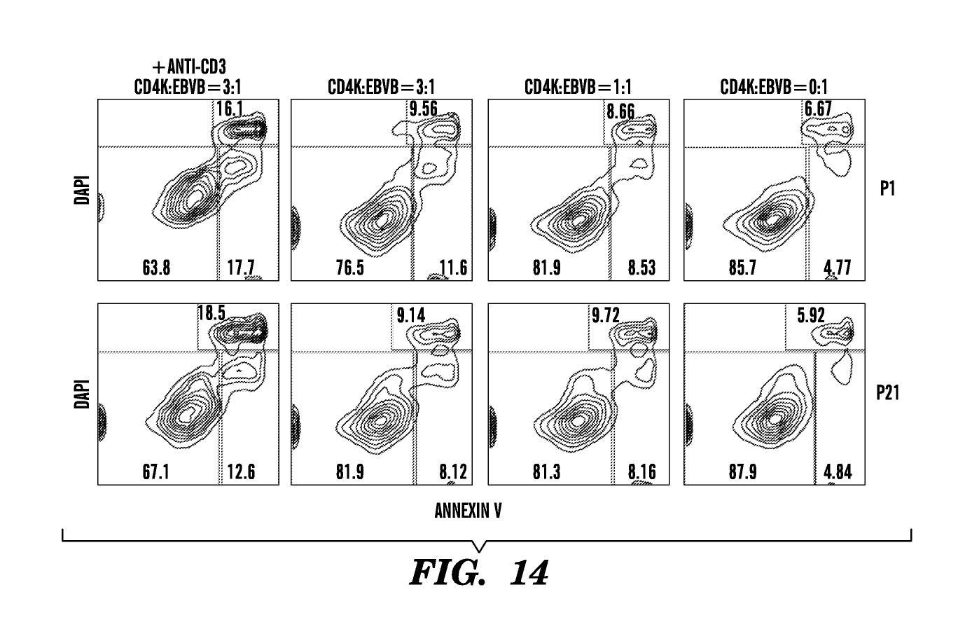

As used herein, the terms “cytotoxic CD4+ T lymphocyte,” “cytotoxic CD4+ T cell” or “cytotoxic CD4+ T cell population” refers to a CD4+ T cell or population thereof having a cell-surface phenotype of CD4+, T-bet+, SLAMF7+, CD11b+, 2B4+, and CD28lo and exhibiting decreased levels of ThPOK and increased expression of Runx3 when compared to CD4+CD45RA+ naïve cells and CD4+CD45RO+ memory cells from healthy controls. These cells also can express surface CD8α and upon in vitro stimulation with anti-CD3, undergo degranulation as inferred from the surface expression of CD107α. These cells also can exhibit cytotoxic activity against co-cultured allogeneic EBV-transformed B cell targets (as described herein in the assays used in FIG. 6G and FIG. 14).

As used herein, the terms “cytotoxic CD4+ T lymphocyte marker” or “T cell marker” refer to a cell-surface or intracellular protein expressed by a cell having a cytotoxic CD4+ T lymphocyte cell-surface phenotype, as described herein. Cytotoxic CD4+ T lymphocyte markers include two or more of CD4, T-bet, SLAMF7, CD11b, 2B4, CD28, perforin, granzyme, ThPOK, and Runx3.

As used herein, SLAMF7, also known as 19A, CS1, CD319, CRACC, refers to a polypeptide having the amino acid sequence of:

(SEQ ID NO: 1)
MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYENNPKGRSSKYGLLHCGNTEKDGKS
PLTAHDARHTKAICL,

as described by, e.g., NP_001269517.1, or

(SEQ ID NO: 2)
MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYEYIEEKKRVDICRETPNICPHSGEN
TEYDTIPHTNRTILKEDPANTVYSTVEIPKKMENPHSLLTMPDTPRLFAY
ENVI,

as described by, e.g., NP_001269518.1, or

(SEQ ID NO: 3)
MAGSPTCLTLIYILWQLTEHLSKPKVTMGLQSNKNGTCVTNLTCCMEHGE
EDVIYTWKALGAANESHNGSILPISWRWGESDMTFICVARNPVSRNFSSP
ILARKLCEGAADDPDSSMVLLCLLLVPLLLSLFVLGLFLWFLKRERQEEY
IEEKKRVDICRETPNICPHSGENTEYDTIPHTNRTILKEDPANTVYSTVE
IPKKMENPHSLLTMPDTPRLFAYENVI,

as described by, e.g., NP_001269519.1, or

(SEQ ID NO: 4)
MAGSPTCLTLIYILWQLTEHLSKPKVTMGLQSNKNGTCVTNLTCCMEHGE
EDVIYTWKALGQAANESHNGSILPISWRWGESDMTFICVARNPVSRNFSS
PILARKLCEEYIEEKKRVDICRETPNICPHSGENTEYDTIPHTNRTILKE
DPANTVYSTVEIPKKMENPHSLLTMPDTPRLFAYENVI,

as described by, e.g., NP_001269520.1, or

(SEQ ID NO: 5)
MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYEHLSKPKVTMGLQSNKNGTCVTNLT
CCMEHGEEDVIYTWKALGQAANESHNGSILPISWRWGESDMTFICVARNP
VSRNFSSPILARKLCEGAADDPDSSMVLLCLLLVPLLLSLFVLGLFLWFL
KRERQEENNPKGRSSKYGLLHCGNTEKDGKSPLTAHDARHTKAICL,

as described by, e.g., NP_001269521.1, or

(SEQ ID NO: 6)
MAGSPTCLTLIYILWQLTEHLSKPKVTMGLQSNKNGTCVTNLTCCMEHGE
EDVIYTWKALGQAANESHNGSILPISWRWGESDMTFICVARNPVSRNFSS
PILARKLCEENNPKGRSSKYGLLHCGNTEKDGKSPLTAHDARHTKAICL,

as described by, e.g., NP_001269522.1,

(SEQ ID NO: 7)
MAGSPTCLTLIYILWQLTEHLSKPKVTMGLQSNKNGTCVTNLTCCMEHGE
EDVIYTWKALGQAANESHNGSILPISWRWGESDMTFICVARNPVSRNFSS
PILARKLCEGDCLSPLHRRLCPGAADDPDSSMVLLCLLLVPLLLSLFVLG
LFLWFLKRERQEEYIEEKKRVDICRETPNICPHSGENTEYDTIPHTNRTI
LKEDPANTVYSTVEIPKKMENPHSLLTMPDTPRLFAYENVI,

as described by, e.g., NP_001269523.1, or

(SEQ ID NO: 8)
MGLQSNKNGTCVTNLTCCMEHGEEDVIYTWKALGQAANESHNGSILPISW
RWGESDMTFICVARNPVSRNFSSPILARKLCEGAADDPDSSMVLLCLLLV
PLLLSLFVLGLFLWFLKRERQEEYIEEKKRVDICRETPNICPHSGENTEY
DTIPHTNRTILKEDPANTVYSTVEIPKKMENPHSLLTMPDTPRLFAYENV
I,

as described by, e.g., NP_001269524.1, or

(SEQ ID NO: 9)
MAGSPTCLTLIYILWQLTEHLSKPKVTMGLQSNKNGTCVTNLTCCMEHGE
EDVIYTWKALGQAANESHNGSILPISWRWGESDMTFICVARNPVSRNFSS
PILARKLCEGAADDPDSSMVLLCLLLVPLLLSLFVLGLFLWFLKRERQEE
NNPKGRSSKYGLLHCGNTEKDGKSPLTAHDARHTKAICL,

as described by, e.g., NP_001269525.1, or

(SEQ ID NO: 10)
MAGSPTCLTLIYILWQLTGSAASGPVKELVGSVGGAVTFPLKSKVKQVDS
IVWTFNTTPLVTIQPEGGTIIVTQNRNRERVDFPDGGYSLKLSKLKKNDS
GIYYVGIYSSSLQQPSTQEYVLHVYEHLSKPKVTMGLQSNKNGTCVTNLT
CCMEHGEEDVIYTWKALGQAANESHNGSILPISWRWGESDMTFICVARNP
VSRNFSSPILARKLCEGAADDPDSSMVLLCLLLVPLLLSLFVLGLFLWFL
KRERQEEYIEEKKRVDICRETPNICPHSGENTEYDTIPHTNRTILKEDPA
NTVYSTVEIPKKMENPHSLLTMPDTPRLFAYENVI,

as described by, e.g., NP_067004.3, together with any other naturally occurring allelic, splice variants, and processed forms (e.g., the mature forms) thereof. Typically, SLAMF7 refers to human SLAMF7. Specific residues of SLAMF7 can be referred to as, for example, “SLAMF7(62).”

As used herein, the terms “SLAMF7 inhibitor,” “SLAMF7 antagonist,” “SLAMF7 inhibitor agent,” and “SLAMF7 antagonist agent” refer to a molecule or agent that significantly blocks, inhibits, reduces, or interferes with SLAMF7 (mammalian, such as a human SLAMF7 of SEQ ID NOs: 1-10) biological activity in vitro, in situ, and/or in vivo, including activity of downstream pathways mediated by SLAMF7 signaling, such as, for example, SLAMF7 mRNA or protein upregulation, and/or elicitation of a cellular response to SLAMF7. Exemplary SLAMF7inhibitors contemplated for use in the various aspects and embodiments described herein include, but are not limited to, anti-SLAMF7 antibodies or antigen-binding fragments thereof that specifically bind to one or more or all SLAMF7 isoforms; anti-sense molecules directed to a nucleic acid encoding SLAMF7; short interfering RNA (“siRNA”) molecules directed to a nucleic acid encoding SLAMF7; a SLAMF7inhibitory compound; RNA or DNA aptamers that bind to one or more or all SLAMF7 isoforms, and inhibit/reduce/block SLAMF7 mediated signaling; SLAMF7 structural analogs; soluble SLAMF7 proteins or fusion polypeptides thereof. In some embodiments of these aspects and all such aspects described herein, a SLAMF7 inhibitor (e.g., an antibody or antigen-binding fragment thereof) binds (physically interacts with) SLAMF7, targets downstream SLAMF7 signaling, and/or inhibits (reduces) SLAMF7 synthesis, production or release. In some embodiments of these aspects and all such aspects described herein, a SLAMF7 inhibitor binds SLAMF7and prevents its binding to its receptor. In some embodiments of these aspects and all such aspects described herein, a SLAMF7 inhibitor specifically reduces or eliminates expression (i.e., transcription or translation) of one or more or all SLAMF7 isoforms. In some embodiments, a SLAMF7 inhibitor indirectly inhibits cytotoxic CD4+ T cells expressing SLAMF7, such as, for example, Rituximab.

As used herein, a SLAMF7 inhibitor or antagonist has the ability to reduce the activity and/or expression of SLAMF7 in a cell (e.g., T cells, such as CD4+ cytotoxic T cells) by at least 5%, at least 10%, at least 20%, at least 30%, at least 40%, at least 50%, at least 60%, at least 70%, at least 80%, at least 90%, at least 95%, at least 98%, at least 99%, or more, relative to the activity or expression level in the absence of the SLAMF7 inhibitor or antagonist.

In some embodiments of the methods and assays described herein, a SLAMF7 inhibitor or antagonist is a monoclonal antibody.

In some embodiments of the compositions, methods, and uses described herein, a SLAMF7 inhibitor or antagonist is an antibody fragment or antigen-binding fragment. The terms “antibody fragment,” “antigen binding fragment,” and “antibody derivative” as used herein, refer to a protein fragment that comprises only a portion of an intact antibody, generally including an antigen binding site of the intact antibody and thus retaining the ability to bind antigen, and as described elsewhere herein.

In some embodiments of the compositions, methods, and uses described herein, a SLAMF7 inhibitor or antagonist is a chimeric antibody derivative of a SLAMF7 antagonist antibody or antigen-binding fragment thereof.

The SLAMF7 inhibitor or antagonist antibodies and antigen-binding fragments thereof described herein can also be, in some embodiments, a humanized antibody derivative.

In some embodiments, the a SLAMF7 inhibitor or antagonist antibodies and antigen-binding fragments thereof described herein, i.e., antibodies that are useful for targeting cytotoxic CD4+ T cells, include derivatives that are modified, i.e., by the covalent attachment of any type of molecule to the antibody, provided that the covalent attachment does not prevent the antibody from binding to the target antigen, e.g., SLAMF7.

In some embodiments of the methods, and uses described herein, completely human antibodies are used, which are particularly desirable for the therapeutic treatment of human patients. A non-limiting example of a publicly available SLAMF7 antibody or antigen-binding fragments thereof that can be used as inhibitory agents in the methods described herein include a humanized monoclonal antibody directed against the human SLAMF7, termed elotuzumab, which has shown promise in patients with advanced multiple myeloma and is being pursued in phase III clinical trials (48).

In some embodiments of the compositions, methods, and uses described herein, a SLAMF7 inhibitor or antagonist is a small molecule inhibitor or antagonist, including, but is not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da. In some embodiments, a SLAMF7 inhibitor or antagonist comprises a small molecule that binds to SLAMF7 and inhibits a SLAMF7 biological activity.

In some embodiments of the compositions, methods, and uses described herein, a SLAMF7 inhibitor or antagonist is an RNA or DNA aptamer that binds or physically interacts with SLAMF7, and blocks interactions between SLAMF7 and its receptor. In some embodiments of the compositions, methods, and uses described herein, a SLAMF7 inhibitor or antagonist is an RNA or DNA aptamer that reduces, impedes, or blocks downstream SLAMF7 signaling.

In some embodiments of the compositions, methods, and uses described herein, a SLAMF7 inhibitor or antagonist comprises at least one SLAMF7 structural analog. The term “SLAMF7 structural analogs,” refer to compounds that have a similar three dimensional structure as part of that of SLAMF7 and which bind to SLAMF7 under physiological conditions in vitro or in vivo, wherein the binding at least partially inhibits a SLAMF7 biological activity. Suitable SLAMF7 structural analogs and can be designed and synthesized through molecular modeling. The SLAMF7 structural analogs can be monomers, dimers, or higher order multimers in any desired combination of the same or different structures to obtain improved affinities and biological effects.

In some embodiments of the compositions, methods, and uses described herein, a SLAMF7 inhibitor or antagonist comprises at least one antisense molecule capable of blocking or decreasing the expression of functional SLAMF7 by targeting nucleic acids encoding SLAMF7. Nucleotide sequences encoding the various SLAMF7 isoforms are known. Methods are known to those of ordinary skill in the art for the preparation of antisense oligonucleotide molecules that will specifically bind one or more or all SLAMF7 isoform mRNAs without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In some embodiment of these aspects and all such aspects described herein, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In some embodiments of the compositions, methods, and uses described herein, a SLAMF7 inhibitor or antagonist comprises at least one siRNA molecule capable of blocking or decreasing the expression of functional SLAMF7 by targeting nucleic acids encoding one or more or all SLAMF7 isoforms. It is routine to prepare siRNA molecules that will specifically target one or more or all SLAMF7 isoform mRNAs without cross-reacting with other polynucleotides. siRNA molecules for use in the compositions, methods, and uses described herein can be generated by methods known in the art, such as by typical solid phase oligonucleotide synthesis, and often will incorporate chemical modifications to increase half life and/or efficacy of the siRNA agent, and/or to allow for a more robust delivery formulation. Alternatively, siRNA molecules are delivered using a vector encoding an expression cassette for intracellular transcription of siRNA.

Other SLAMF7 inhibitors or antagonists for use in the compositions, methods, and uses described herein can be identified or characterized using methods known in the art, such as protein-protein binding assays, biochemical screening assays, immunoassays, and cell-based assays, which are well known in the art, including, but not limited to, those described herein in the Examples.

For example, to identify a molecule that inhibits interaction between SLAMF7 and its receptor, binding assays can be used. For example, a SLAMF7 or receptor polypeptide is immobilized on a microtiter plate by covalent or non-covalent attachment. The assay is performed by adding the non-immobilized component (ligand or receptor polypeptide), which can be labeled by a detectable label, to the immobilized component, in the presence or absence of the testing molecule. When the reaction is complete, the non-reacted components are removed and binding complexes are detected. If formation of binding complexes is inhibited by the presence of the testing molecule, the testing molecule can be deemed a candidate antagonist that inhibits binding between SLAMF7 and its receptor. Cell-based or membrane-based assays can also be used to identify aSLAMF7 antagonists. In other embodiments, by detecting and/or measuring levels of SLAMF7 gene expression, antagonist molecules that inhibit SLAMF7 gene expression can be tested. SLAMF7 gene expression can be detected and/or measured by a variety of methods, such as real time RT-PCR, enzyme-linked immunosorbent assay (“ELISA”), Northern blotting, or flow cytometry, and as known to one of ordinary skill in the art.

IL-1β cytokine is typically produced by activated macrophages as a proprotein, which is proteolytically processed to its active form by caspase 1 (CASP1/ICE). This cytokine is an important mediator of the inflammatory response, and is involved in a variety of cellular activities, including cell proliferation, differentiation, and apoptosis. The induction of cyclooxygenase-2 (PTGS2/COX2) by this cytokine in the central nervous system (CNS) is found to contribute to inflammatory pain hypersensitivity. This gene and eight other interleukin 1 family genes form a cytokine gene cluster on chromosome 2. As used herein, “IL-1β” or “IL-1,” or “IL1F2,” refers to a member of the interleukin 1 cytokine family having the amino acid sequence of:

(SEQ ID NO: 11)
MAEVPELASEMMAYYSGNEDDLFFEADGPKQMKCSFQDLDLCPLDGGIQL
RISDHHYSKGFRQAASVVVAMDKLRKMLVPCPQTFQENDLSTFFPFIFEE
EPIFFDTWDNEAYVHDAPVRSLNCTLRDSQQKSLVMSGPYELKALHLQGQ
DMEQQVVFSMSFVQGEESNDKIPVALGLKEKNLYLSCVLKDDKPTLQLES
VDPKNYPKKKMEKRFVFNKIEINNKLEFESAQFPNWYISTSQAENMPVFL
GGTKGGQDITDFTMQFVSS,

as described by, e.g., NP_000567.1, together with any other naturally occurring allelic, splice variants, and processed forms (e.g., the mature forms) thereof. Typically, IL-1β refers to human IL-1β. Specific residues of IL-1β can be referred to as, for example, “IL-1β (23).”

As used herein, the terms “IL-1β inhibitor,” “IL-1β antagonist,” “IL-1β inhibitor agent,” and “IL-1β antagonist agent” refer to a molecule or agent that significantly blocks, inhibits, reduces, or interferes with IL-1β (mammalian, such as human IL-1β) biological activity in vitro, in situ, and/or in vivo, including activity of downstream pathways mediated by IL-1β signaling, such as, for example, IL-1β mRNA or protein upregulation, and/or elicitation of a cellular response to IL-1β, e.g., inflammasome dependent IL-1β signaling. Exemplary IL-1β inhibitors contemplated for use in the various aspects and embodiments described herein include, but are not limited to, anti-IL-1β antibodies or antigen-binding fragments thereof that specifically bind IL-1β; anti-sense molecules directed to a nucleic acid encoding IL-1β; short interfering RNA (“siRNA”) molecules directed to a nucleic acid encoding IL-1β; a IL-1β inhibitory compound; RNA or DNA aptamers that bind to IL-1β, and inhibit/reduce/block IL-1β mediated signaling; IL-1β structural analogs, such as anakinra; soluble IL-1β proteins or fusion polypeptides thereof. In some embodiments of these aspects and all such aspects described herein, a IL-1β inhibitor (e.g., an antibody or antigen-binding fragment thereof) binds (physically interacts with) IL-1β, targets downstream IL-1β signaling, and/or inhibits (reduces) IL-1β synthesis, production or release. In some embodiments of these aspects and all such aspects described herein, a IL-1β inhibitor binds IL-1β and prevents its binding to its receptor. In some embodiments of these aspects and all such aspects described herein, an IL-1β inhibitor specifically reduces or eliminates expression (i.e., transcription or translation) of IL-1β.

As used herein, a IL-1β inhibitor or antagonist has the ability to reduce the activity and/or expression of IL-1β in a cell (e.g., T cells, such as CD4+ cytotoxic T cells) by at least 5%, at least 10%, at least 20%, at least 30%, at least 40%, at least 50%, at least 60%, at least 70%, at least 80%, at least 90%, at least 95%, at least 98%, at least 99%, or more, relative to the activity or expression level in the absence of the IL-1β inhibitor or antagonist.

In some embodiments of the methods and assays described herein, an IL-1β inhibitor or antagonist is a monoclonal antibody.

In some embodiments of the compositions, methods, and uses described herein, an IL-1β inhibitor or antagonist is an antibody fragment or antigen-binding fragment. The terms “antibody fragment,” “antigen binding fragment,” and “antibody derivative” as used herein, refer to a protein fragment that comprises only a portion of an intact antibody, generally including an antigen binding site of the intact antibody and thus retaining the ability to bind antigen, and as described elsewhere herein.

In some embodiments of the compositions, methods, and uses described herein, an IL-1β inhibitor or antagonist is a chimeric antibody derivative of an IL-1β antagonist antibody or antigen-binding fragment thereof.

The IL-1β inhibitor or antagonist antibodies and antigen-binding fragments thereof described herein can also be, in some embodiments, a humanized antibody derivative.

In some embodiments, the IL-1β inhibitor or antagonist antibodies and antigen-binding fragments thereof described herein, i.e., antibodies that are useful for targeting cytotoxic CD4+ T cells, include derivatives that are modified, i.e., by the covalent attachment of any type of molecule to the antibody, provided that the covalent attachment does not prevent the antibody from binding to the target antigen, e.g., IL-1β.

In some embodiments of the methods, and uses described herein, completely human antibodies are used, which are particularly desirable for the therapeutic treatment of human patients.

Non-limiting examples of a publicly available IL-1β antibody or antigen-binding fragments thereof that can be used as inhibitory agents in the methods described herein include a monoclonal antibody directed against human IL-1β, termed canakinumab, and is a monoclonal antibody that binds to and antagonizes IL-1β and is being studied in clinical trials (48). Other examples of IL-1β inhibitor agents can be found, for example, in U.S. Pat. No. 8,398,966, the contents of which are herein incorporated by reference in their entireties.

In some embodiments of the compositions, methods, and uses described herein, a IL-1β inhibitor or antagonist is a small molecule inhibitor or antagonist, including, but is not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da. In some embodiments, an IL-1β inhibitor or antagonist comprises a small molecule that binds to IL-1β receptor and inhibits an IL-1β biological activity.

In some embodiments of the compositions, methods, and uses described herein, an IL-1β inhibitor or antagonist is an RNA or DNA aptamer that binds or physically interacts with IL-1β, and blocks interactions between IL-1β and its receptor. In some embodiments of the compositions, methods, and uses described herein, an IL-1β inhibitor or antagonist is an RNA or DNA aptamer that reduces, impedes, or blocks downstream IL-1β signaling.

In some embodiments of the compositions, methods, and uses described herein, a IL-1β inhibitor or antagonist comprises at least one IL-1β structural analog. The term “IL-1β structural analogs,” refer to compounds that have a similar three dimensional structure as part of that of IL-1β and which bind to IL-1β under physiological conditions in vitro or in vivo, wherein the binding at least partially inhibits a IL-1β biological activity. Suitable IL-1β structural analogs and can be designed and synthesized through molecular modeling. The IL-1β structural analogs can be monomers, dimers, or higher order multimers in any desired combination of the same or different structures to obtain improved affinities and biological effects.

In some embodiments of the compositions, methods, and uses described herein, a IL-1β inhibitor or antagonist comprises at least one antisense molecule capable of blocking or decreasing the expression of functional IL-1β by targeting nucleic acids encoding IL-1β. Nucleotide sequences encoding IL-1β are known. Methods are known to those of ordinary skill in the art for the preparation of antisense oligonucleotide molecules that will specifically bind IL-1β mRNA without cross-reacting with other polynucleotides. Exemplary sites of targeting include, but are not limited to, the initiation codon, the 5′ regulatory regions, including promoters or enhancers, the coding sequence, including any conserved consensus regions, and the 3′ untranslated region. In some embodiment of these aspects and all such aspects described herein, the antisense oligonucleotides are about 10 to about 100 nucleotides in length, about 15 to about 50 nucleotides in length, about 18 to about 25 nucleotides in length, or more. In certain embodiments, the oligonucleotides further comprise chemical modifications to increase nuclease resistance and the like, such as, for example, phosphorothioate linkages and 2′-O-sugar modifications known to those of ordinary skill in the art.

In some embodiments of the compositions, methods, and uses described herein, a IL-1β inhibitor or antagonist comprises at least one siRNA molecule capable of blocking or decreasing the expression of functional IL-1β by targeting nucleic acids encoding IL-1β. It is routine to prepare siRNA molecules that will specifically target IL-1β mRNA without cross-reacting with other polynucleotides. siRNA molecules for use in the compositions, methods, and uses described herein can be generated by methods known in the art, such as by typical solid phase oligonucleotide synthesis, and often will incorporate chemical modifications to increase half life and/or efficacy of the siRNA agent, and/or to allow for a more robust delivery formulation. Alternatively, siRNA molecules are delivered using a vector encoding an expression cassette for intracellular transcription of siRNA.

Other IL-1β inhibitors or antagonists for use in the compositions, methods, and uses described herein can be identified or characterized using methods known in the art, such as protein-protein binding assays, biochemical screening assays, immunoassays, and cell-based assays, which are well known in the art, including, but not limited to, those described herein in the Examples.

For example, to identify a molecule that inhibits interaction between IL-1β and its receptor, binding assays can be used. For example, IL-1β or its receptor polypeptide is immobilized on a microtiter plate by covalent or non-covalent attachment. The assay is performed by adding the non-immobilized component (ligand or receptor polypeptide), which can be labeled by a detectable label, to the immobilized component, in the presence or absence of the testing molecule. When the reaction is complete, the non-reacted components are removed and binding complexes are detected. If formation of binding complexes is inhibited by the presence of the testing molecule, the testing molecule can be deemed a candidate antagonist that inhibits binding between IL-1β and its receptor. Cell-based or membrane-based assays can also be used to identify IL-1β antagonists. In other embodiments, by detecting and/or measuring levels of IL-1β gene expression, antagonist molecules that inhibit IL-1β gene expression can be tested. IL-1β gene expression can be detected and/or measured by a variety of methods, such as real time RT-PCR, enzyme-linked immunosorbent assay (“ELISA”), Northern blotting, or flow cytometry, and as known to one of ordinary skill in the art.

Antibodies, whether anti-SLAMF7 or anti-IL-1β, suitable for use in practicing the methods described herein are preferably monoclonal, and can include, but are not limited to, human, humanized or chimeric antibodies, comprising single chain antibodies, Fab fragments, F(ab′) fragments, fragments produced by a Fab expression library, and/or binding fragments of any of the above. Antibodies also refer to immunoglobulin molecules and immunologically active portions of immunoglobulin molecules, i.e., molecules that contain antigen or target binding sites or “antigen-binding fragments.” The immunoglobulin molecules described herein can be of any type (e.g., IgG, IgE, IgM, IgD, IgA and IgY), class (e.g., IgG1, IgG2, IgG3, IgG4, IgA1 and IgA2) or subclass of immunoglobulin molecule, as is understood by one of skill in the art.

Examples of antibody fragments encompassed by the terms antibody fragment or antigen-binding fragment include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CH1 domain; (v) the Fv fragment having the V_{L} and V_{H} domains of a single arm of an antibody; (vi) a dAb fragment (Ward et al., Nature 341, 544-546 (1989)) which consists of a V_{H} domain or a V_{L} domain; (vii) isolated CDR regions; (viii) F(ab′)₂ fragments, a bivalent fragment including two Fab′ fragments linked by a disulphide bridge at the hinge region; (ix) single chain antibody molecules (e.g. single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (V_{H}-C_{H}1-V_{H}-C_{H}1) which, together with complementary light chain polypeptides, form a pair of antigen binding regions (Zapata et al. Protein Eng. 8(10):1057-1062 (1995); and U.S. Pat. No. 5,641,870); and modified versions of any of the foregoing (e.g., modified by the covalent attachment of polyalkylene glycol (e.g., polyethylene glycol, polypropylene glycol, polybutylene glycol) or other suitable polymer).

As used herein, the terms “treat” “treatment” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with an immune disease such as an IgG4-RD disease or disorder (e.g., type I autoimmune pancreatitis, Mikulicz's syndrome, Reidel's thyroiditis, retroperitoneal fibrosis, Ki ttner's tumor, sclerosing cholangitis, eosinophilic angiocentric fibrosis, multifocal fibrosclerosis, lymphoplasmacytic sclerosing pancreatitis, autoimmune pancreatitis, inflammatory pseudotumor, fibrosing medistinitis, sclerosing mesenteritis, retroperitoneal fibrosis (Ormond disease), periarteritis/periortitis, inflammatory aortic aneurysm, cutaneous pseudolymphoma, idiopathic hypertrophic pachymeningitis, idiopathic tubulointerstitial nephritis, idiopathic hypocomplementemic tubulointerstitial nephritis with extensive tubulointerstitial deposits, idiopathic cervical fibrosis etc.), among others. Treatment is generally “effective” if one or more symptoms or clinical markers are reduced. Alternatively, treatment is “effective” if the progression of a disease is reduced or halted. That is, “treatment” includes not just the improvement of symptoms or markers, but can also include a cessation or at least slowing of progress or worsening of symptoms that would be expected in absence of treatment. Beneficial or desired clinical results include, but are not limited to, alleviation of one or more symptom(s) of an immune disease, diminishment of extent of the immune disease, stabilized (i.e., not worsening) state of the immune disease, delay or slowing of progression of the disease, amelioration or palliation of the immune disease state, and remission (whether partial or total), whether detectable or undetectable. The term “treatment” of a disease also includes providing relief from the symptoms or side-effects of the disease (including palliative treatment).

In one embodiment, as used herein, the term “prevention” or “preventing” when used in the context of a subject refers to stopping, hindering, and/or slowing down the development of an immune disease and symptoms associated with the immune disease.

As used herein, the term “therapeutically effective amount” means that amount necessary, at least partly, to attain the desired effect, or to delay the onset of, inhibit the progression of, or halt altogether, the onset or progression of the particular disease or disorder being treated (e.g., an immune disease). Such amounts will depend, of course, on the particular condition being treated, the severity of the condition and individual patient parameters including age, physical condition, size, weight and concurrent treatment. These factors are well known to those of ordinary skill in the art and can be addressed with no more than routine experimentation. In some embodiments, a maximum dose of a therapeutic agent is used, that is, the highest safe dose according to sound medical judgment. It will be understood by those of ordinary skill in the art, however, that a lower dose or tolerable dose can be administered for medical reasons, psychological reasons or for virtually any other reason.

In one embodiment, a therapeutically effective amount of a pharmaceutical formulation, or a composition described herein for a method of treating an immune disease is an amount of sufficient to reduce the level of at least one symptom of an immune disease (e.g., pain, inflammation, cytokine production, etc.) as compared to the level in the absence of the compound, the combination of compounds, the pharmaceutical composition/formulation or the composition. In other embodiments, the amount of the composition administered is preferably safe and sufficient to treat, delay the development of an immune disease, and/or delay onset of the immune disease. In some embodiments, the amount can thus cure or result in amelioration of the symptoms of an immune disease, slow the course of the disease, slow or inhibit a symptom of the disease, or slow or inhibit the establishment or development of secondary symptoms of the immune disease. For example, an effective amount of a composition described herein inhibits further pain and/or inflammation associated with an immune disease, cause a reduction in or even completely inhibit pain and/or inflammation associated with an immune disease, even initiate complete regression of the immune disease, and reduce clinical symptoms associated with the immune disease. In one embodiment, an effective amount for treating or ameliorating a disorder, disease, or medical condition is an amount sufficient to result in a reduction or complete removal of the symptoms of the disorder, disease, or medical condition. The effective amount of a given therapeutic agent will vary with factors such as the nature of the agent, the route of administration, the size and species of the animal to receive the therapeutic agent, and the purpose of the administration. Thus, it is not possible or prudent to specify an exact “therapeutically effective amount.” However, for any given case, an appropriate “effective amount” can be determined by a skilled artisan according to established methods in the art using only routine experimentation.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

As used herein, the term “reference value” refers to a reference value, or range of values, obtained for SLAMF7 or another cytotoxic CD4+ T-cell marker from e.g., at least one subject determined to lack a detectable immune disorder. The reference value or range of values can be obtained from a plurality of subjects in a population substantially free of an immune disorder (i.e., is not detectable by typical clinical means) or alternatively from a plurality of subjects in a population having an immune disease. The reference sample can be stored as a value(s) on a computer or PDA device to permit comparison with a value obtained from a subject using the methods described herein. The reference sample can also be obtained from the same subject e.g., at an earlier time point prior to onset of the immune disease or symptoms thereof using clinical tests known to those of skill in the art. One of skill in the art can determine an appropriate reference sample for use with the methods described herein. In one embodiment, the reference is obtained from a subject or plurality of subjects having, or diagnosed with having, an immune disease such as an IgG4-RD disease, type I autoimmune pancreatitis, Mikulicz's syndrome, Reidel's thyroiditis, retroperitoneal fibrosis, Kiittner's tumor, sclerosing cholangitis, etc., among others.

As used herein, the terms “biological sample” refers to a fluid sample, a cell sample, a tissue sample or an organ sample obtained from a subject or patient. Biological samples include, but are not limited to, tissue biopsies, tumor biopsies, scrapes (e.g., buccal scrapes), whole blood, plasma, serum, urine, saliva, cell culture, intestinal lavage, cerebrospinal fluid, circulating tumor cells, and the like. Samples can include frozen or paraffin-embedded tissue. The term “sample” includes any material derived by processing such a sample. Derived samples may, for example, include nucleic acids or proteins extracted from the sample or obtained by subjecting the sample to techniques such as amplification or reverse transcription of mRNA, isolation and/or purification of certain components, etc.

The terms “increased”,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “statistically significant” or “significantly” refers to statistical significance and generally means a two standard deviation (2SD) below normal, or lower, e.g., level of SLAMF7. The term refers to statistical evidence that there is a difference. It is defined as the probability of making a decision to reject the null hypothesis when the null hypothesis is actually true. The decision is often made using the p-value.

As used herein, the terms “free from detectable immune disease” and “substantially free of an immune disease” are used interchangeably and refer to subjects that do not exhibit any clinically detectable signs of an immune disease using routine clinical methods known to those skilled in the art (e.g., routine visual inspection by a health care professional; imaging such as blood screening, ultrasound, CAT scan, endoscopy, CT scan, MRI; palpation; mammogram; routine biopsy, etc).

As used herein, the term “plurality” refers to at least two subjects in a population used to define a reference level of SLAMF7 or another cytotoxic CD4+ T-cell marker, for example, at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, at least 100, at least 125, at least 150, at least 175, at least 200, at least 300, at least 400, at least 500, at least 600, at least 700, at least 800, at least 900, at least 1000, at least 1500, at least 2000, at least 5000, at least 104, at least 105, at least 106, or more subjects in a population.

The term “pharmaceutically acceptable” refers to compounds and compositions which may be administered to mammals without undue toxicity. The term “pharmaceutically acceptable carriers” excludes tissue culture medium. Exemplary pharmaceutically acceptable salts include but are not limited to mineral acid salts such as hydrochlorides, hydrobromides, phosphates, sulfates, and the like, and the salts of organic acids such as acetates, propionates, malonates, benzoates, and the like.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

### Obtaining a Biological Sample

A biological sample can be obtained from essentially any tissue including but not limited to, blood, plasma, serum, circulating cells, brain, liver, lung, gut, stomach, fat, muscle, spleen, testes, uterus, urinary tract, bladder, prostate, esophagus, ovary, skin, endocrine organ and bone, etc. In one embodiment, a biological sample comprises cells including, but not limited to, epithelial, endothelial, neuronal, adipose, cardiac, skeletal muscle, fibroblast, immune cells, hepatic, splenic, lung, circulating blood cells, reproductive cells, gastrointestinal, renal, bone marrow, and pancreatic cells. In one embodiment, the biological sample is a biopsy from a lesion (e.g., a fibrotic lesion, a storiform fibrotic are, a tumefactive lesion etc.).

In one embodiment, the biological sample comprises a tissue biopsy, such as, an aspiration biopsy, a brush biopsy, a surface biopsy, a needle biopsy, a punch biopsy, an excision biopsy, an open biopsy, an incision biopsy or an endoscopic biopsy, or a tumor sample. Biological samples can also be biological fluid samples, including but not limited to, urine, blood, serum, platelets, saliva, cerebrospinal fluid, nipple aspirates, and cell lysate (e.g., supernatant of whole cell lysate, microsomal fraction, membrane fraction, exosomes, or cytoplasmic fraction). Samples can be obtained by any method known to one of skill in the art including e.g., needle biopsy, fine needle aspiration, core needle biopsy, vacuum assisted biopsy, open surgical biopsy, among others.

### Immune Diseases

Essentially any immune disease or disorder can be diagnosed or treated using the methods and compositions described herein. The term “immune disease or disorder” also includes both acute and chronic inflammation.

In some embodiments, the term “immune disease or disorder” refers to diseases and conditions associated with inflammation which include but are not limited to: (1) inflammatory or allergic diseases such as systemic anaphylaxis or hypersensitivity responses, drug allergies, insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis; spondyloarthropathies; systemic sclerosis (e.g., both diffuse and limited variants); localized scleroderma; respiratory allergic diseases such as asthma, allergic rhinitis, hypersensitivity lung diseases, and the like, (2) autoimmune diseases, such as arthritis (rheumatoid and psoriatic), osteoarthritis, multiple sclerosis, systemic lupus erythematosus, diabetes mellitus, glomerulonephritis, and the like, (3) graft rejection (including allograft rejection and graft-v-host disease), and (4) other diseases in which undesired inflammatory responses are to be inhibited (e.g., myositis, inflammatory CNS disorders such as stroke and closed-head injuries, neurodegenerative diseases, Alzheimer's disease, encephalitis, meningitis, osteoporosis, gout, hepatitis, nephritis, sepsis, sarcoidosis, conjunctivitis, otitis, chronic obstructive pulmonary disease, sinusitis and Behcet's syndrome).

In other embodiments, the term “immune disease or disorder” refers to a state of acute or chronic inflammation. An acute inflammatory response is an immediate response by the immune system to a harmful agent. The response includes vascular dilatation, endothelial and neutrophil activation. An acute inflammatory response will either resolve or develop into chronic inflammation.

Chronic inflammation is an inflammatory response of prolonged duration, weeks, months, or even indefinitely, whose extended time course is provoked by the persistence of the causative stimulus to inflammation within the tissue or the development of an autoimmune disorder. The inflammatory process inevitably causes tissue damage. The exact nature, extent and time course of chronic inflammation is variable, and depends on a balance between the causative agent and the attempts of the body to remove it. Agents producing chronic inflammation include, but are not limited to: infectious organisms that can avoid or resist host defenses and so persist in the tissue for a prolonged period; infectious organisms that are not innately resistant but persist in damaged regions where they are protected from host defenses; irritant nonliving foreign material that cannot be removed by enzymatic breakdown or phagocytosis; or where the stimuli is a “normal” tissue component, causing an auto-immune disease. There is a vast array of diseases exhibiting a chronic inflammatory component. These include but are not limited to: inflammatory joint diseases (e.g., rheumatoid arthritis, osteoarthritis, polyarthritis and gout), chronic inflammatory connective tissue diseases (e.g., systemic lupus erythematosus, systemic sclerosis, localized scleroderma, Sjogren's syndrome, poly- and dermatomyositis, vasculitis, mixed connective tissue disease (MCTD), tendonitis, synovitis, bacterial endocarditis, osteomyelitis and psoriasis); chronic inflammatory lung diseases (e.g., chronic respiratory disease, pneumonia, fibrosing alveolitis, chronic bronchitis, bronchiectasis, emphysema, silicosis and other pneumoconiosis and tuberculosis); chronic inflammatory bowel and gastro-intestinal tract inflammatory diseases (e.g., ulcerative colitis and Crohn's disease); chronic neural inflammatory diseases (e.g., chronic inflammatory demyelinating polyradiculoneuropathy, chronic inflammatory demyelinating polyneuropathy, multiple sclerosis, Guillan-Barre Syndrome and myasthemia gravis); other inflammatory diseases (e.g., mastitis, laminitis, laryngitis, chronic cholecystitis, Hashimoto's thyroiditis, inflammatory breast disease); chronic inflammation caused by an implanted foreign body in a wound; and including chronic inflammatory renal diseases including crescentic glomerulonephritis, lupus nephritis, ANCA-associated glomerulonephritis, focal and segmental necrotizing glomerulonephritis, IgA nephropathy, membranoproliferative glomerulonephritis, cryoglobulinaemia and tubulointerstitial nephritis. Diabetic nephropathy may also have a chronic inflammatory component and chronic inflammatory responses are involved in the rejection of transplanted organs. Other non-limiting examples of diseases with symptoms of chronic inflammation include obesity, inflammatory bowel diseases such as Crohn's disease and ulcerative colitis, psoriasis, sarcoidosis, atherosclerosis including plaque rupture, acne rosacea, syphilis, chemical burns, bacterial ulcers, fungal ulcers, Behcet's syndrome, Stevens-Johnson's syndrome, Mycobacteria infections, Herpes simplex infections, Herpes zoster infections, protozoan infections, Mooren's ulcer, leprosy, granulomatosis with polyangiitis (formerly Wegener's granulomatosis), sarcoidosis, pemphigoid, lupus, systemic lupus erythematosus, polyarteritis nodosa, Lyme's disease, Bartonellosis, tuberculosis, histoplasmosis and toxoplasmosis.

Essentially any disease or disorder characterized by, caused by, resulting from, or becoming affected by inflammation can be treated with the methods and compositions described herein.

### Reference Value

As used herein, the terms “reference value” and “reference” refer to the level of SLAMF7, as that term is used herein, in a known sample against which another sample is compared (i.e., obtained from a subject suspected of having an immune disease or disorder). A standard is useful for determining the amount of SLAMF7 (or number of T-cells expressing SLAMF7) or the relative increase/decrease of SLAMF7 or SLAMF7-expressing T-cells in a biological sample. A standard serves as a reference level for comparison, such that samples can be normalized to an appropriate standard in order to infer the presence, absence or extent of an immune disorder in a subject.

In one embodiment, a biological standard is obtained at an earlier time point (presumably prior to the onset of an immune disease) from the same individual that is to be tested or treated as described herein. Alternatively, a standard can be from the same individual having been taken at a time after the onset or diagnosis of such an immune disease. In such instances, the standard can provide a measure of the efficacy of treatment.

A standard level can be obtained, for example, from a known biological sample from a different individual (e.g., not the individual being tested) that is substantially free of an immune disease. A known sample can also be obtained by pooling samples from a plurality of individuals to produce a standard over an averaged population, wherein a standard represents an average level of SLAMF7 (or number or proportion of SLAMF7-expressing T-cells) among a population of individuals. Thus, the level of SLAMF7 in a standard obtained in this manner is representative of an average level of this marker in a general population or a diseased population. An individual sample is compared to this population standard by comparing the level of SLAMF7 from a sample relative to the population standard. Generally, an increase in the amount of SLAMF7 over a standard (e.g., obtained from subjects substantially free of an immune disease) will indicate the presence of an immune disease, while a decrease in the amount of SLAMF7 will indicate no immune disease is present. The converse is contemplated in cases where a standard is obtained from a population of subjects having an immune disease. It should be noted that there is often variability among individuals in a population, such that some individuals will have higher levels of SLAMF7, while other individuals have lower levels of SLAMF7. However, one skilled in the art can make logical inferences on an individual basis regarding the detection and treatment of the immune disease as described herein.

A standard or series of standards can also be synthesized. A known amount of SLAMF7 (or a series of known amounts) can be prepared within the typical range for SLAMF7 that is observed in a general population. This method has an advantage of being able to compare the extent of disease in two individuals in a mixed population. This method can also be useful for subjects who lack a prior sample to act as a standard or for routine follow-up post-diagnosis. This type of method can also allow standardized tests to be performed among several clinics, institutions, or countries etc.

### Antibodies

In one embodiment, a therapeutic antibody that binds to e.g., SLAMF7 and/or another cytotoxic CD4+ T-cell marker are used herein in the treatment of an IgG4-related disease. In another embodiment, one or more antibodies that bind to SLAMF7 and/or another cytotoxic CD4+ T-cell marker are used herein to determine the amount of SLAMF7 or the amount/number of SLAMF7-expressing T-cells in a biological sample obtained from a subject.

An “antibody” that can be used according to the methods described herein includes complete immunoglobulins, antigen binding fragments of immunoglobulins, as well as antigen binding proteins that comprise antigen binding domains of immunoglobulins. Antigen binding fragments of immunoglobulins include, for example, Fab, Fab′, F(ab′)2, scFv and dAbs. Modified antibody formats have been developed which retain binding specificity, but have other characteristics that may be desirable, including for example, bispecificity, multivalence (more than two binding sites), and compact size (e.g., binding domains alone). Single chain antibodies lack some or all of the constant domains of the whole antibodies from which they are derived. Therefore, they can overcome some of the problems associated with the use of whole antibodies. For example, single-chain antibodies tend to be free of certain undesired interactions between heavy-chain constant regions and other biological molecules. Additionally, single-chain antibodies are considerably smaller than whole antibodies and can have greater permeability than whole antibodies, allowing single-chain antibodies to localize and bind to target antigen-binding sites more efficiently. Furthermore, the relatively small size of single-chain antibodies makes them less likely to provoke an unwanted immune response in a recipient than whole antibodies. Multiple single chain antibodies, each single chain having one VH and one VL domain covalently linked by a first peptide linker, can be covalently linked by at least one or more peptide linker to form multivalent single chain antibodies, which can be monospecific or multispecific. Each chain of a multivalent single chain antibody includes a variable light chain fragment and a variable heavy chain fragment, and is linked by a peptide linker to at least one other chain. The peptide linker is composed of at least fifteen amino acid residues. The maximum number of linker amino acid residues is approximately one hundred. Two single chain antibodies can be combined to form a diabody, also known as a bivalent dimer Diabodies have two chains and two binding sites, and can be monospecific or bispecific. Each chain of the diabody includes a VH domain connected to a VL domain. The domains are connected with linkers that are short enough to prevent pairing between domains on the same chain, thus driving the pairing between complementary domains on different chains to recreate the two antigen-binding sites. Three single chain antibodies can be combined to form triabodies, also known as trivalent trimers. Triabodies are constructed with the amino acid terminus of a VL or VH domain directly fused to the carboxyl terminus of a VL or VH domain, i.e., without any linker sequence. The triabody has three Fv heads with the polypeptides arranged in a cyclic, head-to-tail fashion. A possible conformation of the triabody is planar with the three binding sites located in a plane at an angle of 120 degrees from one another. Triabodies can be monospecific, bispecific or trispecific. Thus, antibodies useful in the methods described herein include, but are not limited to, naturally occurring antibodies, bivalent fragments such as (Fab′)2, monovalent fragments such as Fab, single chain antibodies, single chain Fv (scFv), single domain antibodies, multivalent single chain antibodies, diabodies, triabodies, and the like that bind specifically with an antigen.

Antibodies can also be raised against a polypeptide or portion of a polypeptide by methods known to those skilled in the art. Antibodies are readily raised in animals such as rabbits or mice by immunization with the gene product, or a fragment thereof. Immunized mice are particularly useful for providing sources of B cells for the manufacture of hybridomas, which in turn are cultured to produce large quantities of monoclonal antibodies. Antibody manufacture methods are described in detail, for example, in Harlow et al., 1988. While both polyclonal and monoclonal antibodies can be used in the methods described herein, it is preferred that a monoclonal antibody is used where conditions require increased specificity for a particular protein.

Useful monoclonal antibodies and fragments can be derived from any species (including humans) or can be formed as chimeric proteins which employ sequences from more than one species. Human monoclonal antibodies or “humanized” murine antibodies are also used in accordance with the methods and assays described herein. For example, a murine monoclonal antibody can be “humanized” by genetically recombining the nucleotide sequence encoding the murine Fv region (i.e., containing the antigen binding sites) or the complementarily determining regions thereof with the nucleotide sequence encoding a human constant domain region and an Fc region. Humanized targeting moieties are recognized to decrease the immunoreactivity of the antibody or polypeptide in the host recipient, permitting an increase in the half-life and a reduction of the possibly of adverse immune reactions. The murine monoclonal antibodies should preferably be employed in humanized form. Antigen binding activity is determined by the sequences and conformation of the amino acids of the six complementarily determining regions (CDRs) that are located (three each) on the light and heavy chains of the variable portion (Fv) of the antibody. The 25-kDa single-chain Fv (scFv) molecule is composed of a variable region (VL) of the light chain and a variable region (VH) of the heavy chain joined via a short peptide spacer sequence. Techniques have been developed to display scFv molecules on the surface of filamentous phage that contain the gene for the scFv. scFv molecules with a broad range of antigenic-specificities can be present in a single large pool of scFv-phage library.

Chimeric antibodies are immunoglobin molecules characterized by two or more segments or portions derived from different animal species. Generally, the variable region of the chimeric antibody is derived from a non-human mammalian antibody, such as murine monoclonal antibody, and the immunoglobin constant region is derived from a human immunoglobin molecule. In some embodiments, both regions and the combination have low immunogenicity as routinely determined

### Dosage and Administration

In some aspects, the methods described herein provide a method for treatment an immune disease (e.g., IgG4-RD spectrum disorders such as type I autoimmune pancreatitis, Mikulicz's syndrome, Reidel's thyroiditis, retroperitoneal fibrosis, Kiittner's tumor, and sclerosing cholangitis, among others) or inhibiting cytotoxic CD4+ T cells in a subject. In another embodiment of this aspect and all other aspects described herein, the immune disease is an IgG4-RD disease or disorder. In one embodiment, the subject can be a mammal. In another embodiment, the mammal can be a human, although the approach is effective with respect to all mammals. The methods comprise administering to the subject an effective amount of a pharmaceutical composition comprising an inhibitor that binds SLAMF7, IL-1β, or a combination thereof in a pharmaceutically acceptable carrier.

The dosage range for the agent depends upon the potency, and includes amounts large enough to produce the desired effect, e.g., immune response modulation. The dosage should not be so large as to cause unacceptable adverse side effects. Generally, the dosage will vary with the type of inhibitor (e.g., an antibody or fragment, small molecule, siRNA, etc.), and with the age, condition, and sex of the patient. The dosage can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

A therapeutically effective amount is an amount of an agent that is sufficient to produce a statistically significant, measurable change in immune response (see “Efficacy Measurement” below). Such effective amounts can be gauged in clinical trials as well as animal studies for a given agent.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. In one embodiment it is preferred that the agents for the methods described herein are administered directly to a lesion (e.g., during surgery or by direct injection). The agent can be administered systemically, if so desired.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. In some embodiments, an agent can be targeted to a tissue by using bispecific antibodies, for example produced by chemical linkage of an anti-ligand antibody (Ab) and an Ab directed toward a specific target. To avoid the limitations of chemical conjugates, molecular conjugates of antibodies can be used for production of recombinant bispecific single-chain Abs directing ligands and/or chimeric inhibitors at cell surface molecules. The addition of an antibody to an agent permits the agent to accumulate additively at the desired target site (e.g., lesion). Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

### Pharmaceutical Compositions

The present invention includes, but is not limited to, therapeutic compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

### Efficacy Measurement

The efficacy of a given treatment for an immune disease (e.g., IgG4-RD spectrum disease or disorder, type I autoimmune pancreatitis, Mikulicz's syndrome, Reidel's thyroiditis, retroperitoneal fibrosis, Kiittner's tumor, sclerosing cholangitis, among others) can be determined by the skilled clinician. However, a treatment is considered “effective treatment,” as the term is used herein, if any one or all of the signs or symptoms of the immune disease is/are altered in a beneficial manner, other clinically accepted symptoms or markers of disease are improved, or even ameliorated, e.g., by at least 10% following treatment with an agent that comprises an inhibitor that bind SLAMF7. Efficacy can also be measured by failure of an individual to worsen as assessed by stabilization of the immune disease, hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the immune disease; or (2) relieving the disease, e.g., causing regression of symptoms; and (3) preventing or reducing the likelihood of the development of the immune disease, or preventing secondary diseases/disorders associated with the immune disease (e.g., scarring, tumors, cancer metastasis).

An effective amount for the treatment of a disease means that amount which, when administered to a mammal in need thereof, is sufficient to result in effective treatment as that term is defined herein, for that disease. Efficacy of an agent can be determined by assessing physical indicators of the immune disease, such as e.g., redness, pain, inflammation, lung capacity, size of lesions, tumor growth rate, mobility of subject, etc.

### Systems

Embodiments of the invention also provide for systems (and computer readable media for causing computer systems) to perform a method for diagnosing an immune disease or disorder in a subject, or assessing a subject's risk of developing such a disease or disorder.

Embodiments of the invention can be described through functional modules, which are defined by computer executable instructions recorded on computer readable media and which cause a computer to perform method steps when executed. The modules are segregated by function for the sake of clarity. However, it should be understood that the modules/systems need not correspond to discreet blocks of code and the described functions can be carried out by the execution of various code portions stored on various media and executed at various times. Furthermore, it should be appreciated that the modules may perform other functions, thus the modules are not limited to having any particular functions or set of functions.

The computer readable storage media #30 can be any available tangible media that can be accessed by a computer. Computer readable storage media includes volatile and nonvolatile, removable and non-removable tangible media implemented in any method or technology for storage of information such as computer readable instructions, data structures, program modules or other data. Computer readable storage media includes, but is not limited to, RAM (random access memory), ROM (read only memory), EPROM (eraseable programmable read only memory), EEPROM (electrically eraseable programmable read only memory), flash memory or other memory technology, CD-ROM (compact disc read only memory), DVDs (digital versatile disks) or other optical storage media, magnetic cassettes, magnetic tape, magnetic disk storage or other magnetic storage media, other types of volatile and non-volatile memory, and any other tangible medium which can be used to store the desired information and which can accessed by a computer including and any suitable combination of the foregoing.

Computer-readable data embodied on one or more computer-readable storage media may define instructions, for example, as part of one or more programs that, as a result of being executed by a computer, instruct the computer to perform one or more of the functions described herein, and/or various embodiments, variations and combinations thereof. Such instructions may be written in any of a plurality of programming languages, for example, Java, J#, Visual Basic, C, C#, C++, Fortran, Pascal, Eiffel, Basic, COBOL assembly language, and the like, or any of a variety of combinations thereof. The computer-readable storage media on which such instructions are embodied may reside on one or more of the components of either of a system, or a computer readable storage medium described herein, may be distributed across one or more of such components.

The computer-readable storage media can be transportable such that the instructions stored thereon can be loaded onto any computer resource to implement the aspects of the present invention(s) discussed herein. In addition, it should be appreciated that the instructions stored on the computer-readable medium, described above, are not limited to instructions embodied as part of an application program running on a host computer. Rather, the instructions can be embodied as any type of computer code (e.g., software or microcode) that can be employed to program a computer to implement aspects of the present invention. The computer executable instructions can be written in a suitable computer language or combination of several languages. Basic computational biology methods are known to those of ordinary skill in the art and are described in, for example, Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The functional modules of certain embodiments of the invention(s) include at minimum a determination system #40, a storage device #30, a comparison module #80, and a display module #110. The functional modules can be executed on one, or multiple, computers, or by using one, or multiple, computer networks. The determination system has computer executable instructions to provide e.g., SLAMF7 expression information in computer readable form.

The determination system #40, can comprise any system for detecting a signal representing the level of SLAMF7. Such systems can include colorimetric assays, flow cytometry, immunocytochemistry, assays etc.

The information determined in the determination system can be read by the storage device #30. As used herein the “storage device” is intended to include any suitable computing or processing apparatus or other device configured or adapted for storing data or information. Examples of electronic apparatus suitable for use with the present invention include stand-alone computing apparatus, data telecommunications networks, including local area networks (LAN), wide area networks (WAN), Internet, Intranet, and Extranet, and local and distributed computer processing systems. Storage devices also include, but are not limited to: magnetic storage media, such as floppy discs, hard disc storage media, magnetic tape, optical storage media such as CD-ROM, DVD, electronic storage media such as RAM, ROM, EPROM, EEPROM and the like, general hard disks and hybrids of these categories such as magnetic/optical storage media. The storage device is adapted or configured for having recorded thereon values representing levels of SLAMF7 information. Such information may be provided in digital form that can be transmitted and read electronically, e.g., via the Internet, on diskette, via USB (universal serial bus) or via any other suitable mode of communication.

As used herein, “stored” refers to a process for encoding information on the storage device. Those skilled in the art can readily adopt any of the presently known methods for recording information on known media to generate manufactures comprising expression information.

In one embodiment the reference data stored in the storage device to be read by the comparison module is e.g., SLAMF7 expression data obtained from a population of subjects that are substantially free of immune disease.

The “comparison module” #80 can use a variety of available software programs and formats for the comparison operative to compare sequence information data determined in the determination system to reference samples and/or stored reference data. In one embodiment, the comparison module is configured to use pattern recognition techniques to compare information from one or more entries to one or more reference data patterns. The comparison module can be configured using existing commercially-available or freely-available software for comparing patterns, and may be optimized for particular data comparisons that are conducted. The comparison module provides computer readable information related to the amount of SLAMF7 or number of SLAMF7-expressing T-cells present in a biological sample obtained from a subject.

The comparison module, or any other module of the invention, can include an operating system (e.g., UNIX) on which runs a relational database management system, a World Wide Web application, and a World Wide Web server. World Wide Web application includes the executable code necessary for generation of database language statements (e.g., Structured Query Language (SQL) statements). Generally, the executables will include embedded SQL statements. In addition, the World Wide Web application may include a configuration file which contains pointers and addresses to the various software entities that comprise the server as well as the various external and internal databases which must be accessed to service user requests. The Configuration file also directs requests for server resources to the appropriate hardware--as may be necessary should the server be distributed over two or more separate computers. In one embodiment, the World Wide Web server supports a TCP/IP protocol. Local networks such as this are sometimes referred to as “Intranets.” An advantage of such Intranets is that they allow easy communication with public domain databases residing on the World Wide Web (e.g., the GenBank or Swiss Pro World Wide Web site). Thus, in one embodiment of the methods described herein, users can directly access data (via Hypertext links for example) residing on Internet databases using a HTML interface provided by Web browsers and Web servers.

The comparison module provides a computer readable comparison result that can be processed in computer readable form by predefined criteria, or criteria defined by a user, to provide a content based in part on the comparison result that can be stored and output as requested by a user using a display module #110.

The content based on the comparison result, can be data relating to the amount of SLAMF7 or number of SLAMF7-expressing T-cells in a biological sample indicating the presence or absence of an immune disease in a subject.

In one embodiment of the invention, the content based on the comparison result is displayed on a computer monitor #120. In one embodiment of the invention, the content based on the comparison result is displayed through printable media #130, #140. The display module can be any suitable device configured to receive from a computer and display computer readable information to a user. Non-limiting examples include, for example, general-purpose computers such as those based on Intel PENTIUM-type processor, Motorola PowerPC, Sun UltraSPARC, Hewlett-Packard PA-RISC processors, any of a variety of processors available from Advanced Micro Devices (AMD) of Sunnyvale, California, or any other type of processor, visual display devices such as flat panel displays, cathode ray tubes and the like, as well as computer printers of various types.

In one embodiment, a World Wide Web browser is used for providing a user interface for display of the content based on the comparison result. It should be understood that other modules of the systems described herein can be adapted to have a web browser interface. Through the Web browser, a user may construct requests for retrieving data from the comparison module. Thus, the user will typically point and click to user interface elements such as buttons, pull down menus, scroll bars and the like conventionally employed in graphical user interfaces.

The methods described herein therefore provide for systems (and computer readable media for causing computer systems) to perform methods for diagnosing an immune disease or assessing risk for developing such a disorder in a subject.

Systems and computer readable media described herein are merely illustrative embodiments of the invention(s) described herein for performing methods of diagnosis in an individual, and are not intended to limit the scope of the invention. Variations of the systems and computer readable media described herein are possible and are intended to fall within the scope of the invention.

The modules of the machine, or those used in the computer readable medium, may assume numerous configurations. For example, function may be provided on a single machine or distributed over multiple machines.

It is understood that the foregoing description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

All patents and other publications identified are expressly incorporated herein by reference for the purpose of describing and disclosing, for example, the methodologies described in such publications that could be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Study Summary

Background: IgG4-related disease (IgG4-RD) is a poorly understood, multi-organ, chronic inflammatory disease characterized by storiform fibrosis, tumefactive lesions and elevated plasma IgG4 levels. Direct evidence implicating T cells has been lacking, although Th2 cells have been assumed to be important in pathogenesis. The mechanism of clinical improvement following B-cell depletion with rituximab remains unclear, since IgG4 is regarded as a non-inflammatory immunoglobulin.

Methods: We searched for clonally expanded B and T cells in the peripheral blood of subjects with active, untreated, biopsy-proven IgG4-RD using next-generation sequencing of antigen receptor genes and by single cell PCR and sequencing. RNA expression profiles of clonally expanded CD4+CD45RO+CD27loCD62Llo effector/memory T (TEM or T_{EM}) cells were obtained from subjects whose clinical presentations varied widely. Clonally expanded CD19+CD27+CD38hi plasmablasts and TEM cells were monitored by flow cytometry after rituximab-mediated B-cell depletion therapy.

Results: Clonal expansions of TEM cells and plasmablasts were found in IgG4-RD subjects. TEM cells from subjects with different clinical presentations also expressed T-bet, CD11b, perform, granzyme B, and secreted both IL-1β and IFN-γ. SLAMF7, a cell-surface protein that is not expressed on T cells from healthy controls helped define both the TEM and plasmablast expansions.

Conclusions: These studies implicate clonal SLAMF7-expressing T_{EM} cells in the pathogenesis of IgG4-RD. SLAMF7 represents a rational therapeutic target for IgG4-RD.

### EXAMPLE 1: B Cell-Targeted Therapy Depletes Clonally-Expanded Circulating CD4+ Effector/Memory T Cells in IgG4-Related Disease

IgG4-related disease (IgG4-RD) is a multi-organ chronic inflammatory condition characterized by tumefactive lesions, storiform fibrosis, and mild to moderate tissue eosinophilial, a lymophoplasmacytic infiltrate rich in IgG4+ plasma cells and frequently elevated serum IgG4 concentration¹⁻³. IgG4-RD includes subjects previously diagnosed with other disorders that were typically defined by the dominant pattern of organ involvement. Examples of such diagnoses that are now classified as part of the IgG4-RD spectrum are type I autoimmune pancreatitis, Mikulicz's syndrome, Reidel's thyroiditis, retroperitoneal fibrosis, Küttner's tumor, and sclerosing cholangitis, among others⁴⁻⁶. The clinical manifestations of this syndrome have been reviewed elsewhere². Little is known about the pathogenesis of IgG4-RD, but autoantibodies have been identified in subsets of subjects⁷⁻⁹ and depletion of B cells with rituximab results in striking clinical improvement¹⁰.

There are conflicting reports that restimulated circulating CD4+ T cells can produce IFNγ and not IL-4 and vice versa in IgG4-RD⁹⁻¹¹. Cytokines such as IL-4 and IL-10 have also been reported within tissue lesions¹²⁻¹³ and it is likely that CD4+ T cells play a role in disease pathogenesis¹⁴. However, these cytokines can be secreted by a number of cell types other than CD4+ T cells, including innate immune cells, innate lymphoid cells and B cells. Direct evidence implicating CD4+ T cells is lacking.

Studies in rodents have demonstrated that B cells maintain CD4+ memory T cells¹⁷⁻¹⁸. We considered the possibility that B-cells in IgG4-RD subjects sustain specific clonally-expanded T_{EM} cells, which are the true drivers of the disease process.

Autoantibodies have been described in the majority of subjects with type 1 autoimmune pancreatitis^{8,9,19}, and oligoclonal expansion of IgG4+ B cells has been inferred by next-generation sequencing of immunoglobulin (Ig) heavy (H) chain genes in subjects with IgG4-related sclerosing cholangitis²⁰. Given that IgG4-RD is marked by the predominance of a single immunoglobulin isotype, we analyzed antigen receptor gene usage in circulating activated B and T cells using next generation sequencing and single cell polymerase chain reaction (PCR) approaches. We reasoned that the identification of clonal expansions of disease causing effector CD4+ T cells would allow the determination of the specific T-cell-derived cytokines and effector molecules that drive the aberrant activation of innate immune cells, which in turn induce the fibrotic phenotype of IgG4-RD.

Methods All studies were approved by the Human Studies Institutional Review Board at the Massachusetts General Hospital.

Peripheral blood mononuclear cells were isolated using endotoxin-free Ficoll-PaquePLUS (GE HEALTHCARE) density-gradient centrifugation. Cell surface staining was performed using fluorophore-conjugated antibodies against IgG4, CD19, CD38, CD27, CD4, CD45RO, and CD127. Specific TCR Vβ antibodies and lymphocyte sub-populations were analyzed or sorted using by flow cytometry (BD LSR II, BD FACS ARIA III).

Individual IgG4+ plasmablasts were sorted into a 96-well plate. Using primers specific for the IgG4 heavy chain, paired IgG4 heavy (IgH) and light chain sequences from single cells were determined using single cell polymerase chain reaction (PCR) and Sanger sequencing. Next-generation sequencing of IgH chains and TCRβ chains was performed on flow-sorted plasmablasts and CD4+ effector/memory populations, respectively, using the IMMUNOSEQ platform from ADAPTIVE BIOTECHNOLOGIES INC. Non-productive rearrangements were excluded from the analysis. Gene expression analysis was performed on effector and naive T cells from IgG4-RD subjects and healthy controls using an nCounter panel human immunology-related gene array (NANOSTRING TECHNOLOGIES). Biopsies were analyzed for gene expression using in situ hybridization and immunofluorescence approaches.

Results: Immunological analyses were conducted on subjects with IgG4-RD and controls. All disease subjects had multi-organ disease, elevated serum IgG4 concentrations, and biopsy-proven IgG4-RD with tumefactive lesions characterized by storiform fibrosis.

Clonal Expansion of plasmablasts in IgG4-RD: In general, flow cytometry examination of the peripheral blood was performed prior to therapeutic intervention and then at ten days, one month, and three months following the initiation of therapy. Since preliminary gene expression and flow cytometric studies revealed the expression of SLAMF7 specifically on disease-related effector memory CD4+ T cells, a finding that has potential therapeutic significance, plasmablasts from patients were also interrogated for SLAMF7 expression. Plasmablasts from IgG4-RD subjects expressed high levels of SLAMF7 as well.

### Clonal Expansion of T_{EM} Cells in IgG4-RD

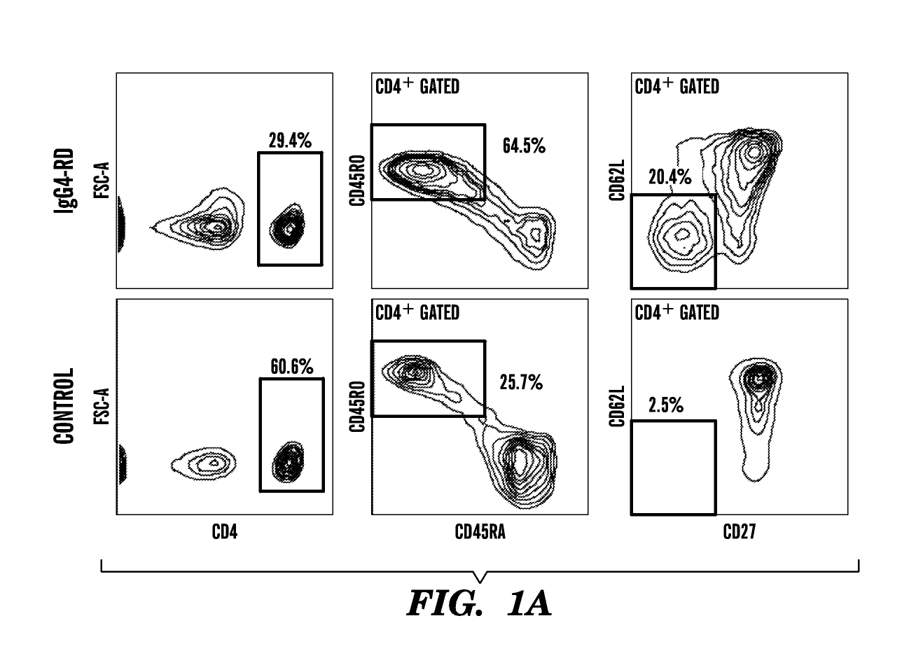

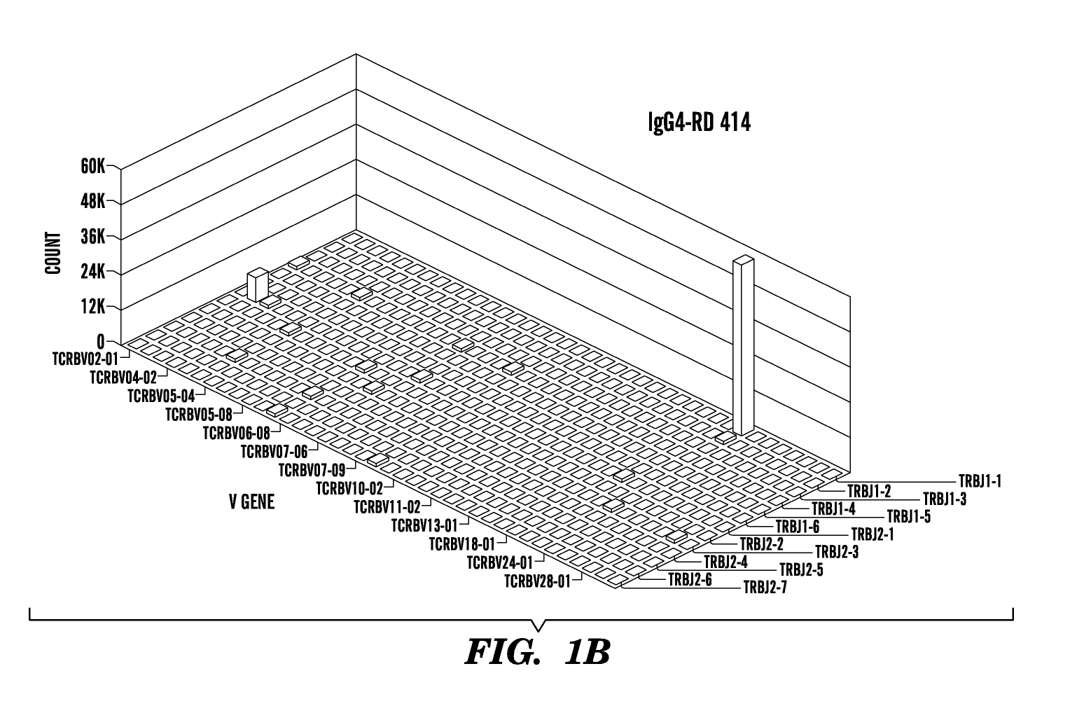

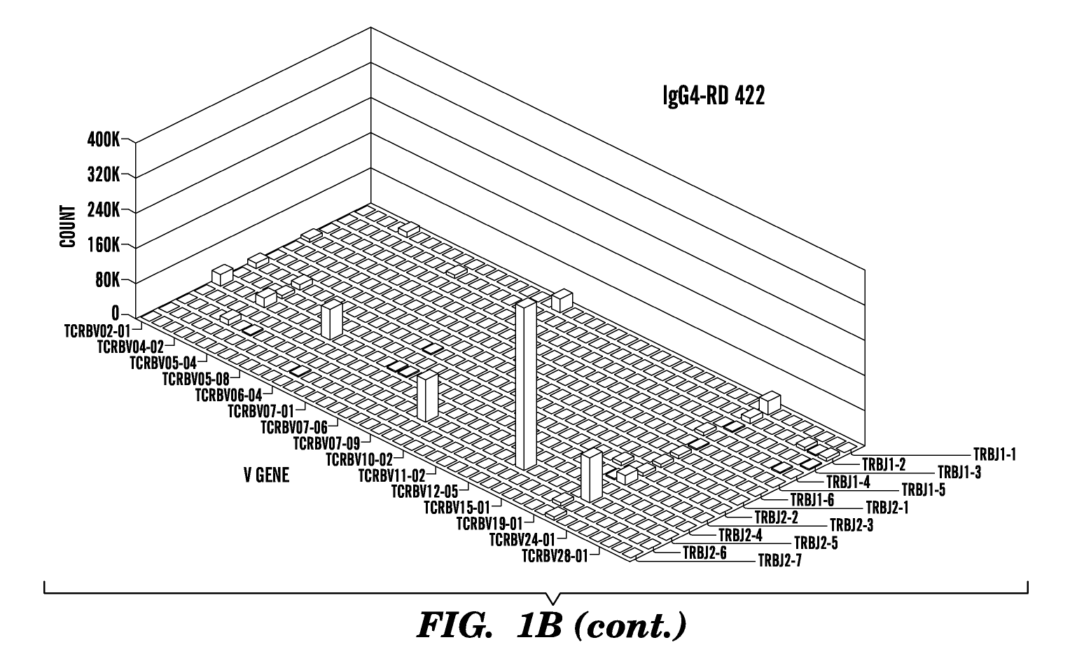

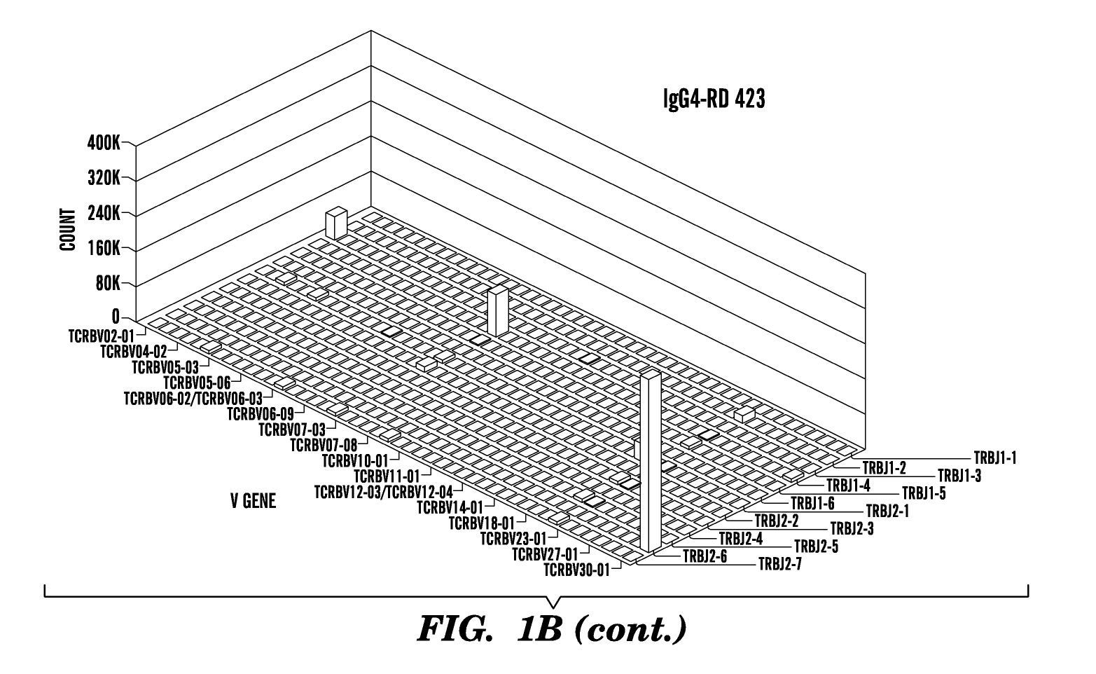

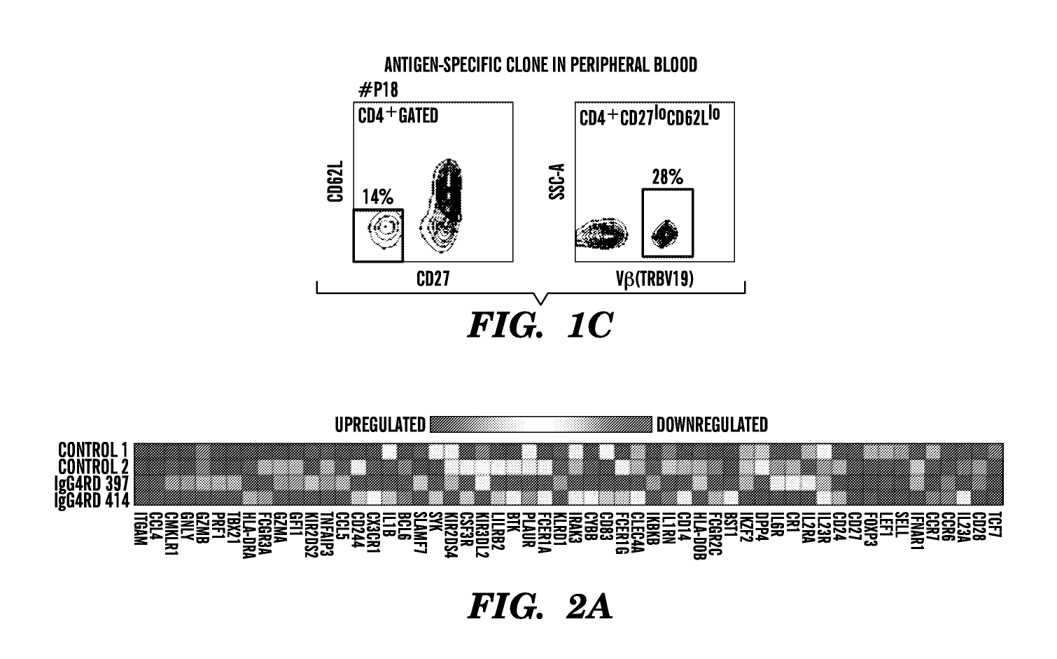

T cells are abundant in the lymphocytic infiltrate in IgG4-RD tissue lesions²¹. In addition, both the immunoglobulin class switch and the somatic mutations that characterize the humoral immune response in IgG4-RD are T-cell dependent. We therefore investigated CD4+ T-cell populations in subjects with active IgG4-RD affecting different organs and identified an expansion of antigen-experienced CD4⁺CD45RO⁺ T cells, especially the CD27^{lo}CD62L^{lo} TEM subset, in the majority of IgG4-RD subjects as shown for a representative index case (FIG. 1A). FoxP3-expressing regulatory T cells and T follicular helper cells were not prominent in the circulation of IgG4-RD subjects with active disease (data not shown). Next-generation sequencing of the TCRIβ repertoire of TEM cells from untreated subjects with active disease revealed oligoclonal expansions of TEM cells, typically with a single dominant clone (FIG. 1B). Several minor expanded clones were also seen in each patient. Disease-related T_{EM} clones detected by next-generation sequencing were validated against TCR Vβ as shown in an index case for whom Vβ-specific antibodies were available (FIG. 1C

### Gene Expression Profiling and Flow Cytometry Defines the Disease-Related TEM Cell Pool as CD4+T-bet+SLAMF7+CD11b+2B4+CD28lo Cells

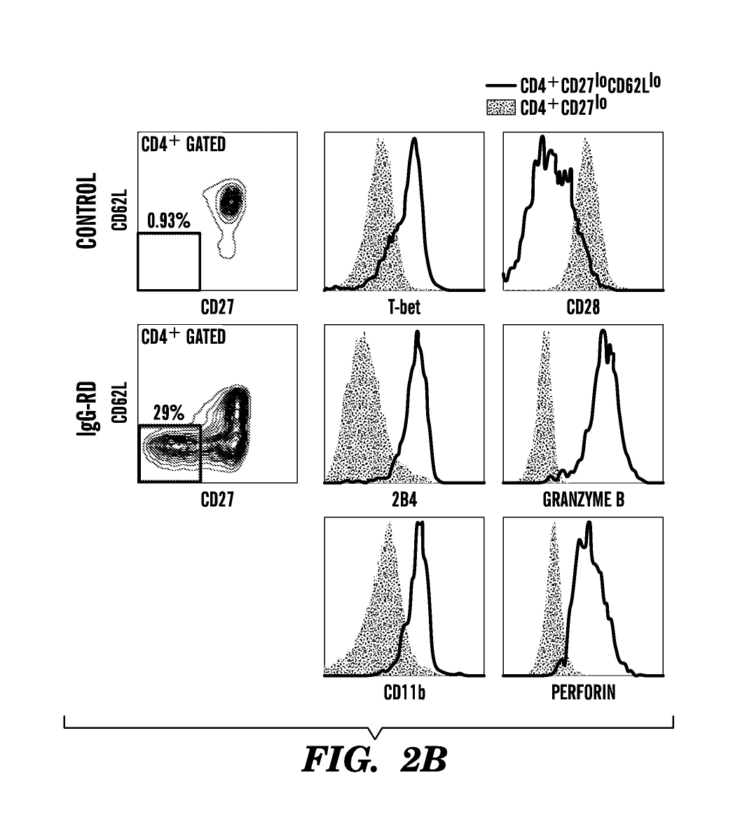

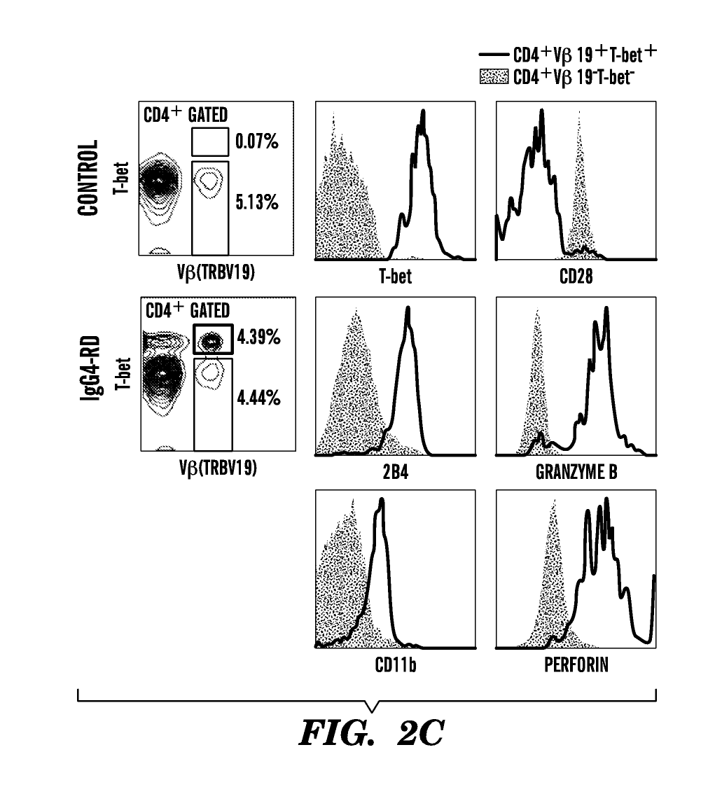

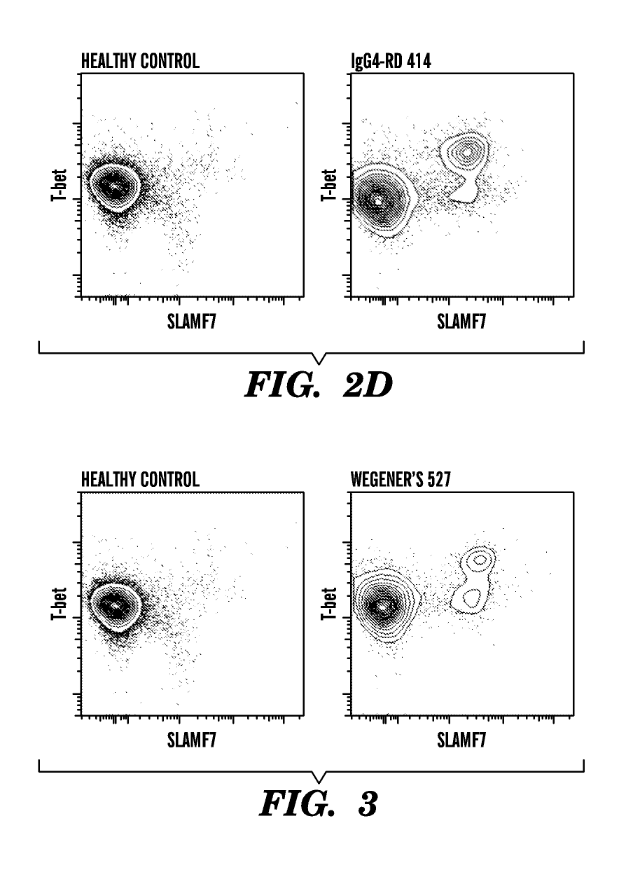

Flow-sorted T_{EM} cells from subjects with active disease but different clinical presentations and distinct dominant organ involvement were compared by expression profiling of 458 immune-related genes. Total CD4+CD45RO+ cells from IgG4-RD subjects and healthy controls were used for comparison. We identified a disease-specific pattern of gene expression in the expanded T_{EM} cells that was conserved across different disease subjects (FIG. 2A). We specifically searched for genes encoding membrane proteins that were uniquely expressed in the expanded T_{EM} cells. One surface protein that fit this criterion was SLAMF7. These cells therefore comprise a T_{EM} subset with a unique expression profile that includes specific markers such as SLAMF7 with the co-expression of CD11b, 2B4, granzyme B, perform, and the T-bet transcription factor, and loss of CD28. We validated these findings by flow cytometry in IgG4-RD subjects (FIG. 2B).These markers were also confirmed on clonally-expanded TEM cells identified using TCR Vβ-specific antibodies (FIG. 2C).

SLAMF7 is a unique marker of this disease-related T_{EM} population (FIG. 2D). This protein is not expressed on naïve CD4+ T cells from IgG4-RD subjects, nor is it expressed on naïve or memory CD4+ T cells from healthy controls. Furthermore, it is not expressed on circulating B cells in healthy individuals but is expressed on plasmablasts in IgG4-RD subjects. This protein therefore represents an ideal therapeutic target for IgG4-RD and possibly for other inflammatory or fibrotic disorders, as well. SLAMF7 has also been found on T_{EM} cells from a patients with Wegener's disease (FIG. 3) supporting the notion of its link to other fibrotic and inflammatory diseases as well. These latter disease include a range of autoimmune diseases including, but not limited to, systemic lupus erythematosus, rheumatoid arthritis, multiple sclerosis, type I diabetes and others.

This study illustrates several novel and clinically relevant findings. Oligoclonal expansions of circulating SLAMF7+ plasmablasts and clonal CD4+ Tbet+SLAMF7+CD11b+2B4+T_{EM} cells are seen in IgG4-RD, especially in active disease. These T-cell clones are unusual in terms of their ability to make both IFN-γ and IL-1β, the latter being a cytokine typically thought to be made by non-lymphoid cells²⁵. The expanded T cell clones are also found in affected tissues and the decline in their numbers following rituximab therapy parallels the observed clinical improvement, implicating these T cells in disease pathogenesis. The production of large amounts of IFN-γ and IL-1β by these T cells following brief restimulation, the expression of pre-formed cytotoxic mediators, and the strong correlation of this subset with disease activity indicates that the CD4+Tbet+SLAMF7+CD11b+2B4+T_{EM} population is largely comprised of CD4+ effector T cells that drive this disease. These cells may have been presumed to be Th1 effectors in prior studies on account of their expression of T-bet and IFN-γ.

Another finding of therapeutic relevance is the striking observation that both the effector T-cell clones and plasmablasts in IgG4-RD subjects express a plasma membrane protein of the SLAM family, SLAMF7, which is absent on naive T cells in IgG4-RD subjects and on naive or memory T or B cells in healthy controls. Thus, SLAMF7 represents a highly specific potential therapeutic target and therapeutic antibodies against SLAMF7 are already being evaluated in a clinical trial for multiple myeloma^{28,29}. Our observations indicate that this antibody selectively targets the two types of cells that appear central to the pathophysiology of IgG4-RD, and represents a more direct approach to treating IgG4-RD than the depletion of CD20-expressing B cells.

Fibrosis is an essential feature of the histopathology of IgG4-RD, and the fibrosis has a characteristic “storiform” morphology²¹. However, the driver of fibrosis in IgG4-RD remains unclear. Our observations show that clonally-expanded CD4+Tbet+SLAMF7+CD11b+2B4+ T_{EM} cells express granzyme B, perform, IFN-γ and IL1-β are prominent in this disease. Transient expression of IL1-β alone in the rat lung has been shown to result in tissue damage and progressive fibrosis³⁰. Transgenic overexpression of IL-1(3 in the murine pancreas results in a fibrotic pancreatitis³¹. IL-1R1/MyD88 signaling and the inflammasome are essential drivers of the fibrotic process in the widely studied murine model of pulmonary fibrosis induced by bleomycin³². IFN-γ has been shown to contribute to fibrosis in a murine thyroiditis model³³. These CD4+Tbet+SLAMF7+CD11b+2B4+ TEM cells serve as an abundant source of both IFN-γ and IL-1β in the tissue lesions of IgG4-RD, thereby potentially driving fibrosis.

CD4+CD28^{lo} cells, which have been shown to express cytotoxic mediators and IFN-γ in some studies, are observed in idiopathic pulmonary fibrosis, severe rheumatoid arthritis, and granulomatosis with polyangiitis (formerly Wegener's granulomatosis)³⁴⁻³⁶. The CD4+Tbet+SLAMF7+CD11b+2B4+ TEM cells that we have identified in IgG4-RD also express reduced levels of CD28 and may be related to these previously described CD4+CD28^{lo} cells. Thus, targeting SLAMF7-expressing cells represents a rational therapeutic strategy in other immune-mediated conditions associated with severe tissue damage and fibrosis. A survey of SLAM family protein expression in systemic lupus erythematosus revealed higher levels of SLAMF7 in some B- and T cells, but the cell phenotypes were not characterized further³⁷. Studies in a wide range of diseases examining the prevalence of similar CD4+T-bet+SLAMF7+CD11b+2B4+ T cells are currently being undertaken.

CD4+Tbet+SLAMF7+CD11b+2B4+ effector T cells producing IFN-γ and IL1-β are present in all IgG4-RD subjects examined so far and their numbers strongly correlate with disease activity. Although Th2 cells have been previously implicated in IgG4-RD in some studies¹¹⁻¹³, we have not seen a consistent expansion of Th2 effector cells in active IgG4-RD. Instead, we find that Th2 central memory cells are particularly expanded in a subset of IgG4-RD patients with a history of chronic allergies, a frequent clinical finding in this disease. Thus, IgG4-RD subjects can be divided into two groups based on the degree of expansion of Th2 memory cells. Given the presumed role of Th2 cells in IgG4 class-switching, there are clinical and pathogenic implications of such sub-groupings.

Without wishing to be bound or limited by theory, one scenario is that the clonally-expanded CD4+T-bet+SLAMF7+CD11b+2B4+ effector T cells drive the inflammatory process in IgG4-RD, whereas Th2 memory cells expressing IL-4 induce isotype switching to the non-inflammatory IgG4 subclass. IgG4 antibodies are generally considered to be non-inflammatory since they do not efficiently engage activating Fc receptors and complement and can be functionally monovalent in vivo³⁹. We have observed active disease in subjects with relatively low levels of plasma IgG4 but a clear expansion of IgG4+ plasmablasts and of the CD4+T-bet+SLAMF7+CD11b+2B4+ T effector cell population. The participation, if at all, of IgG4 antibodies in disease pathogenesis is therefore unclear. Without wishing to be bound by theory, it is possible that the IgG4 response seen in disease represents an exaggerated but ineffectual attempt to dampen inflammation. Alternatively, and without wishing to be bound or limited by theoryIgG4 antibodies and IgG4+ plasmablasts may contribute in some way to the fibrotic disease process by mechanisms that are yet to be elucidated.

### REFERENCES

1. Stone JH, Zen Y, Deshpande V. IgG4-related disease. N Engl J Med 2012;366:539-51.

2. Hamano H, Kawa S, Horiuchi A, et al. High serum IgG4 concentrations in patients with sclerosing pancreatitis. N Engl J Med 2001;344:732-8.

3. Kamisawa T, Egawa N, Nakajima H. Autoimmune pancreatitis is a systemic autoimmune disease. Am J Gastroenterol 2003;98:2811-2.

4. Stone JH, Khosroshahi A, Deshpande V, et al. Recommendations for the nomenclature of IgG4-related disease and its individual organ system manifestations. Arthritis Rheum 2012;64:3061-7.

5. Umehara H, Okazaki K, Masaki Y, et al. A novel clinical entity, IgG4-related disease (IgG4RD): general concept and details. Mod Rheumatol 2012;22:1-14.

6. Khosroshahi A, Stone JH. A clinical overview of IgG4-related systemic disease. Curr Opin Rheumatol 2011;23:57-66.

7. Aoki S, Nakazawa T, Ohara H, et al Immunohistochemical study of autoimmune pancreatitis using anti-IgG4 antibody and patients' sera. Histopathology 2005;47:147-58.

8. Nishimori I, Miyaji E, Morimoto K, Nagao K, Kamada M, Onishi S. Serum antibodies to carbonic anhydrase IV in patients with autoimmune pancreatitis. Gut 2005;54:274-81.

9. Okazaki K, Uchida K, Ohana M, et al. Autoimmune-related pancreatitis is associated with autoantibodies and a Th1/Th2-type cellular immune response. Gastroenterology 2000;118:573-81

10. Khosroshahi A, Bloch DB, Deshpande V, Stone JH. Rituximab therapy leads to rapid decline of serum IgG4 levels and prompt clinical improvement in IgG4-related systemic disease. Arthritis Rheum 2010;62:1755-62.

11. Kanari H, Kagami S, Kashiwakuma D, et al. Role of Th2 cells in IgG4-related lacrimal gland enlargement. Int Arch Allergy Immunol 2010;152 Supp! 1:47-53.

12. Zen Y, Fujii T, Harada K, et al. Th2 and regulatory immune reactions are increased in immunoglobin G4-related sclerosing pancreatitis and cholangitis. Hepatology 2007;45:1538-46.

13. Tanaka A, Moriyama M, Nakashima H, et al. Th2 and regulatory immune reactions contribute to IgG4 production and the initiation of Mikulicz disease. Arthritis Rheum 2012;64:254-63.

14. Zen Y, Nakanuma Y. Pathogenesis of IgG4-related disease. Curr Opin Rheumatol 2011;23:114-8.

15. Ouyang W, Rutz S, Crellin NK, Valdez PA, Hymowitz SG. Regulation and functions of the IL-10 family of cytokines in inflammation and disease. Annu Rev Immunol 2011;29:71-109.

16. Gessner A, Mohrs K, Mohrs M. Mast cells, basophils, and eosinophils acquire constitutive IL-4 and IL-13 transcripts during lineage differentiation that are sufficient for rapid cytokine production. J Immunol 2005;174:1063-72.

17. Crawford A, Macleod M, Schumacher T, Corlett L, Gray D. Primary T cell expansion and differentiation in vivo requires antigen presentation by B cells. J Immunol 2006;176:3498-506

18. Whitmire JK, Asano MS, Kaech SM, et al. Requirement of B cells for generating CD4+ T cell memory. J Immunol 2009;182:1868-76.

19. Frulloni L, Lunardi C, Simone R, et al. Identification of a novel antibody associated with autoimmune pancreatitis. N Engl J Med 2009;361:2135-42.

20. de Buy Wenniger LJ, Doorenspleet ME, Klarenbeek PL, et al. IgG4+ clones identified by next-generation sequencing dominate the b-cell receptor repertoire in IgG4-associated cholangitis. Hepatology 2013.

21. Deshpande V, Zen Y, Chan JK, et al. Consensus statement on the pathology of IgG4-related disease. Mod Pathol 2012;25:1181-92.

22. Sallusto F, Lenig D, Forster R, Lipp M, Lanzavecchia A. Two subsets of memory T lymphocytes with distinct homing potentials and effector functions. Nature 1999;401:708-12.

23. Khosroshahi A, Carruthers MN, Deshpande V, Unizony S, Bloch DB, Stone JH. Rituximab for the treatment of IgG4-related disease: lessons from 10 consecutive patients. Medicine (Baltimore) 2012;91:57-66.

24. Carruthers MN, Stone JH, Deshpande V, Khosroshahi A. Development of an IgG4-RD Responder Index. Int J Rheumatol 2012;2012:259408.

25. Dinarello CA. Immunological and inflammatory functions of the interleukin-1 family. Annu Rev Immunol 2009;27:519-50.

26. Barr TA, Shen P, Brown S, et al. B cell depletion therapy ameliorates autoimmune disease through ablation of IL-6-producing B cells. J Exp Med 2012;209:1001-10.

27. Rock KL, Benacerraf B, Abbas AK. Antigen presentation by hapten-specific B lymphocytes. I. Role of surface immunoglobulin receptors. J Exp Med 1984;160:1102-13.

28. Benson DM, Jr., Byrd JC. CS1-directed monoclonal antibody therapy for multiple myeloma. J Clin Oncol 2012;30:2013-5.

29. Hsi ED, Steinle R, Balasa B, et al. CS1, a potential new therapeutic antibody target for the treatment of multiple myeloma. Clin Cancer Res 2008;14:2775-84.

30. Kolb M, Margetts PJ, Anthony DC, Pitossi F, Gauldie J. Transient expression of IL-1beta induces acute lung injury and chronic repair leading to pulmonary fibrosis. J Clin Invest 2001;107:1529-36.

31. Marrache F, Tu SP, Bhagat G, et al. Overexpression of interleukin-1beta in the murine pancreas results in chronic pancreatitis. Gastroenterology 2008;135:1277-87.

32. Gasse P, Mary C, Guenon I, et al. IL-1R1/MyD88 signaling and the inflammasome are essential in pulmonary inflammation and fibrosis in mice. J Clin Invest 2007;117:3786-99.

33. Chen K, Wei Y, Sharp GC, Braley-Mullen H. Balance of proliferation and cell death between thyrocytes and myofibroblasts regulates thyroid fibrosis in granulomatous experimental autoimmune thyroiditis (G-EAT). Journal of leukocyte biology 2005;77:166-72.

34. Gilani SR, Vuga LJ, Lindell KO, et al. CD28 down-regulation on circulating CD4 T-cells is associated with poor prognoses of patients with idiopathic pulmonary fibrosis. PLoS One 2010;5:e8959.

35. Komocsi A, Lamprecht P, Csernok E, et al. Peripheral blood and granuloma CD4(+)CD28(-) T cells are a major source of interferon-gamma and tumor necrosis factor-alpha in Wegener's granulomatosis. Am J Pathol 2002;160:1717-24.

36. Martens PB, Goronzy JJ, Schaid D, Weyand CM. Expansion of unusual CD4+ T cells in severe rheumatoid arthritis. Arthritis Rheum 1997;40:1106-14.

37. Kim JR, Mathew SO, Patel RK, Pertusi RM, Mathew PA. Altered expression of signalling lymphocyte activation molecule (SLAM) family receptors CS1 (CD319) and 2B4 (CD244) in patients with systemic lupus erythematosus. Clin Exp Immunol 2010;160:348-58.

38. Aalberse RC, Schuurman J. IgG4 breaking the rules. Immunology 2002;105:9-19.

39. Aalberse RC, Stapel SO, Schuurman J, Rispens T. Immunoglobulin G4: an odd antibody. Clin Exp Allergy 2009;39:469-77.

### Example 2: Additional Methods

Patients: Informed consent was obtained from serially encountered patients with IgG4-RD referred to or presenting at the rheumatology clinic of the Massachusetts General Hospital. Patients with active untreated IgG4-RD were chosen for this study. 15 mL of peripheral blood was collected in EDTA or ACD tubes (BD VACUTAINER) and transported to the laboratory for cell isolation on the same day.

Isolation of mononuclear cells: Mononuclear cells were isolated from peripheral blood of IgG4-RD subjects and healthy controls by FICOLL-PLAQUE PLUS (GE HEALTHCARE) density-gradient centrifugation following the manufacturer's protocol. To facilitate subsequent analysis of cells in batches, mononuclear cells were resuspended in fetal bovine serum containing 10% dimethyl sulfoxide and cryopreserved in vapor phase liquid nitrogen.

Histology and immunofluorescence: A biopsy of the enlarged submandibular salivary gland was fixed and processed for hematoxylin and eosin staining, and immunohistochemical detection. Anti-human TCR Vβ19 conjugated to PE clone ELL1.4, BECKMAN-COULTER) was used for immunofluorescence detection of clonally-expanded T cells Immunohistochemistry and immunofluorescence was done using previously published protocols.

Flow cytometric analysis: Fluorescence labeling for flow cytometry was performed by incubating cells in staining buffer (BIOLEGEND) containing optimized concentrations of fluorochrome-conjugated antibodies. Except where indicated, all antibodies were procured from BIOLEGEND. The following monoclonal antibodies were used in this study: anti-human CD19-Pacific Blue (clone HIB19), anti-human CD27-APC (clone O323), anti-human CD38-FITC (clone HIT2), anti-human IgG4 (clone 6025, SOUTHERN BIOTECH), anti-human CD4-PECy7 (clone OKT4), anti-human-CD45RA-PE (clone HI100), anti-human CD45RO-APC (clone UCHL1), anti-human CD62L-FITC (clone DREG56), anti-human CD244-biotin (clone C1.7, EBIOSCIENCE), anti-human CD319(SLAMF7)-PE (clone 162.1), anti-human CD11b-APC Cy7 (clone ICRF44), anti-human CD28-PerCP Cy5.5 (clone CD28.2), anti-human TCR V1323 FITC (clone αHUT7), anti-human TCR Vβ19-PE (clone ELL1.4, BECKMAN-COULTER), anti-human TCR Vβ2-PE (clone MPB2D5, BECKMAN-COULTER). A table of concordance between the TCR Vβ gene nomenclatures and IMGT gene names was used to verify that the appropriate Vβ-specific antibody clones were selected to detect clonally-expanded T-cell clones identified by next-generation sequencing¹. For intracellular staining of transcription factors (T-bet, GATA-3 and Foxp3) as well as cytolytic molecules (granzyme B and perforin) cells were fixed and permeabilized with the Foxp3-staining kit (EBIOSCIENCE) according to manufacturer's guidelines. Cells were then stained in permeabilization buffer with anti-human T-bet PECy7 (clone 4B10, EBIOSCIENCE), anti-human GATA-3 PECy7 (clone L50-823, BD BIOSCIENCES), anti-human Foxp3 (clone 206D, BIOLEGEND), anti-human Perforin PE (clone B-D48) and anti-human granzyme B FITC (clone GB11).

Intracellular staining for cytokines in restimulated T cells: For detecting intracellular levels of cytokines, mononuclear cells were stimulated with 100 ng/mL of phorbol-myristoyl acetate (SIGMA-ALDRICH) and 100 ng/mL of ionomycin (1NVITROGEN) in the presence of Brefeldin A (SIGMA-ALDRICH) for 4 hrs at 37° C. They were subsequently labeled with the LIVE/DEAD fixable violet viability dye (INVITROGEN) in phosphate-buffered saline for 20 minutes and stained for cell surface markers. Cells were then fixed/permeabilized and stained with anti-human IFN-γ APC (clone 4S.B3), anti-human IL-4 ALEXA FLUOR® 488 (clone 8D4-8) for 45 minutes on ice. Cells were washed two times with permeabilization buffer and once with PBS and acquired/analyzed on a BD LSR II (BD BIOSCIENCES). The fcs files were analyzed using FLOWJO software (version 9.6.3, TREESTAR).

Cell sorting: To preserve cell viability mononuclear cells were stained with relevant cell surface markers in DMEM (GIBCO) with ITS+ Universal Culture Supplement (BD BIOSCIENCES). For batch sorts, antibody-stained cells were resuspended at ˜20 million/mL in HBSS (GIBCO) plus 10 mM glucose and sorted on a BD FACSARIA II (BD BIOSCIENCES). Sorted cells were collected in 5 mL tubes containing 1 mL collection medium (DMEM with 30% FBS) and re-analyzed on the sorter to ensure >99% purity in defined gates. For single plasmablast sorting, cells were collected in 96-well PCR plates (VWR) containing 4 μL of cell lysis buffer (0.5X PBS containing 10 mM DTT and 8 U RNasin (PROMEGA) and the plates were stored at −80° C. to preserve RNA integrity.

Single-cell PCR and sequencing: Single-cell PCR and sequencing of individually sorted plasmablasts was carried out with minor modifications to the method described by Tiller et al². cDNA was synthesized in the original 96-well PCR plate, containing individual plasmablasts in each well, using 150 ng phosphorylated random hexamers (pd(N)6, AMERSHAM PHARMACIA BIOTECH), 0.5 μL dNTP-Mix (10 mM each nucleotide), 1 μl 0.1 M DTT, 0.5% v/v NP40, 8 U RNAse Inhibitors and 50 U Superscript III reverse transcriptase (INVITROGEN) at 37° C. for 55 minutes. 3.5 μl of cDNA or 1st-PCR products were used to amplify IgH (using degenerate IgVH forward primers and a ss), Igλ or Igk transcripts (using degenerate forward IgVκ/Vλ and reverse Cκ/Cλ primers) by two successive rounds of PCR in 20 μl reactions containing 20 pM primers and 1.2 U Hotstart Taq DNA polymerase (QIAGEN). Each round of PCR was performed for 50 cycles at 94° C. for 30 sec, 57° C. (IgH/Igκ) or 60° C. (Igλ) for 30 sec, 72° C. for 55 sec (1st-PCR) or 45 sec (2nd PCR). Except for the IgG4-CH outer reverse primer (AGGGCGCCAGGGGGAAGACG) (SEQ ID NO: 12), the primer sequences used were identical to those described by Tiller et al². Successful PCR amplification was possible from >80% of individually sorted plasmablasts. All PCR products were purified (QIAQUICK PCR purification kit, QIAGEN), sequenced and analyzed on the V-Quest server³.

Next generation sequencing analysis of BCR IgH and TCR Vβ repertoire: Next-generation sequencing analysis of BCR IgH and TCR Vβ repertoire was undertaken using the IMMUNOSEQ® platform at ADAPTIVE BIOTECHNOLOGIES™ Inc. at the “survey” level of sequencing depth, that is designed to target an output of 200,000 assembled output sequences after preprocessing filters^{4,5}. Briefly, genomic DNA was isolated from flow-sorted plasmablasts or effector/memory CD4+ T cells from individual IgG4-RD subjects. Sorted cell numbers ranged from ˜5,000 to ˜40,000, assuring at least 5-fold depth of sequencing. Genomic rearrangements of V-D-J gene segments at the TCRβ and IgH loci were amplified using multiplex PCR with forward and reverse primers specific for V and J gene segments respectively, and analyzed by paired-end ILLUMINA sequencing. Use of barcoded primers allowed multiplexing of next-generation sequencing samples on the ILLUMINA HI-SEQ instrument. The sequences were assembled in silico, and V-D-J regions were reconstructed, following standard IMGT gene nomenclature for BCR Ig VH and TCR Vβ gene segments¹. Non-productive rearrangements were excluded from the analysis. Sequence assembly and initial bioinformatic analysis was performed by ADAPTIVE BIOTECHNOLOGIES™. Analysis of somatic hypermutation in the rearranged IGH sequences was performed by the authors using the V-Quest server and BASELINe (version 1.1)^{3.6}.

Gene-expression analysis: The nCounter® human immunology panel (NANOSTRING Technologies™), comprising ˜500 immunology-related genes was used to quantify the gene expression of effector/memory T cells in IgG4-RD. RNA was extracted from 10,000-50,000 flow-sorted T cells using the QIAGEN RNAEASY MICRO kit according to manufacturer's protocol. Targets were reverse-transcribed and pre-amplified for 14 cycles using the standard multiplexed target enrichment (MTE) protocol (NANOSTRING TECHNOLOGIES™) for 458 target genes, which has been previously validated to yield a linear response. The amplified products were hybridized in solution to color-coded NCOUNTER capture and reporter probes and captured on an NCOUNTER Cartridge for high-resolution digital scanning and analysis on the GEN2 Digital Analyzer at NANOSTRING TECHNOLOGIES™. The raw gene expression data was normalized to the mean of the spiked-in internal positive control probes to correct for technical assay variation and subsequently normalized to the mean of 15 housekeeping genes included in the NCOUNTER codeset to correct for differences in sample input or variation in reverse-transcription/pre-amplification. Biological replicates were evaluated for consistency and differential expression analysis of gene expression was undertaken using the ComparativeMarkerSelection module in the GenePattern pipeline (version 3.5.0)^{7.8}.

ELISpot for IL1-β: Prior to coating, 96-well polyvinylidene difluoride (PVDF) membrane plates (MILLIPORE) were prewet with 50 ul 70% ethanol/well for 2 minutes and washed three times with 200 ul sterile filtered water. ELISPOT assay for IL-1β was performed using human IL-1β ELISPOT READY-SET-GO kit (EBIOSCIENCES) according to the manufacturer's recommendations. In brief, plates were coated overnight at 4° C. with the anti-human IL-1β antibody provided, followed by gentle washing with ELISpot wash buffer (1X PBS plus 0.05% Tween-20) and blocking with complete medium (RPMI plus 10% fetal bovine serum) for 2 hours at room temperature. 10000 sorted CD4+CD45RO+ cells from healthy controls and IgG4-RD patients were rested on the plate in complete medium for 4 hours post-sorting, and stimulated with PMA (100 ng/ml) and ionomycin (100 ng/ml) overnight at 37° C. Cells were decanted gently using wash buffer to detach any adherent cells and plates were washed three times with wash buffer followed by incubation with the recommended dilution of biotinylated detection antibody for 2 hours at room temperature. After two additional washes, plates were incubated with horse-radish peroxidase conjugated with streptavidin for 45 minutes at room temperature. The plates were washed extensively 4 times with wash buffer followed by two additional washes with PBS to remove any traces of tween-20. 100 μL of TMB substrate (MABTECH) was added and spots were allowed to develop for up to 20 minutes. Counting and visual analysis of the spots were done using a computer-operated CTL ELISpot reader and the fraction of IL-1β secreting cells was quantified as the number of spots detected per 10,000 cells applied to the well.

HLA typing: Genomic DNA was isolated from PBMCs from 1-2 mL of whole blood from 24 IgG4-RD subjects using a QIAGEN WHOLE BLOOD DNA MIDI kit. HLA class I and class II alleles were typed to a 4-digit resolution at the Carrington lab, NIH, using a multiplexed next-generation sequencing protocol⁹. This method is based on the use of the FLUIDIGM ACCESS ARRAY System, and allows multi-locus PCR amplification using 14 locus-specific primer pairs (covering exons 2, 3, and 4 of the class I loci and exons 2 of DRBI, 3/4/5, DQA1, DQB1, DPB1, and exon 3 of DQB1), in combination with a unique multiplex identifier for each subject for next-generation sequencing. Pooled exonic amplicons were sequenced using the 454 Life Sciences GS FLX System. HLA genotypes were assigned to each patient using the CONEXIO software.

IgG4-RD Responder Index: The IgG4-RD Responder Index is a clinical measure of outcome and disease activity¹⁰. It is based on clinical assessment, imaging and diagnostic test results, developed along the lines of the Birmingham Vasculitis Activity Score, specifically for use in IgG4-RD subjects. The IgG4-RD Responder Index was calculated for all patients for whom adequate clinical data was available.

### REFERENCES

1. Lefranc MP, Pommie C, Ruiz M, et al. IMGT unique numbering for immunoglobulin and T cell receptor variable domains and Ig superfamily V-like domains. Dev Comp Immunol 2003;27:55-77.

2. Tiller T, Meffre E, Yurasov S, Tsuiji M, Nussenzweig MC, Wardemann H. Efficient generation of monoclonal antibodies from single human B cells by single cell RT-PCR and expression vector cloning. J Immunol Methods 2008;329:112-24.

3. Brochet X, Lefranc MP, Giudicelli V. IMGT/V-QUEST: the highly customized and integrated system for IG and TR standardized V-J and V-D-J sequence analysis. Nucleic Acids Res 2008;36:W503-8.

4. Larimore K, McCormick MW, Robins HS, Greenberg PD. Shaping of human germline IgH repertoires revealed by deep sequencing. J Immunol 2012;189:3221-30.

5. Robins HS, Campregher PV, Srivastava SK, et al. Comprehensive assessment of T-cell receptor beta-chain diversity in alpha beta T cells. Blood 2009;114:4099-107.

6. Yaari G, Uduman M, Kleinstein SH. Quantifying selection in high-throughput Immunoglobulin sequencing data sets. Nucleic Acids Res 2012;40:e134.

7. Gould J, Getz G, Monti S, Reich M, Mesirov JP. Comparative gene marker selection suite. Bioinformatics 2006;22:1924-5.

8. Reich M, Liefeld T, Gould J, Lerner J, Tamayo P, Mesirov JP. GenePattern 2.0. Nat Genet 2006;38:500-1.

9. Moonsamy PV, Williams T, Bonella P, et al. High throughput HLA genotyping using 454 sequencing and the Fluidigm Access Array System for simplified amplicon library preparation. Tissue Antigens 2013;81:141-9.

10. Carruthers MN, Stone JH, Deshpande V, Khosroshahi A. Development of an IgG4-RD Responder Index. Int J Rheumatol 2012;2012:259408.

### Example 3

Fibrosis is almost always a consequence of inflammation. It can involve virtually any organ and is a prominent feature of many chronic inflammatory disorders, including rheumatoid arthritis, systemic sclerosis, systemic lupus erythematosus and IgG4-related disease among others. Many distinct triggers are known to contribute to fibrosis, but a detailed understanding of this pathological process has proved elusive (1).

Both innate and adaptive immune mechanisms may drive fibrotic responses (2) but it is unclear what constitutes the tipping point between physiological wound healing and pathological fibrosis. Activated macrophages secrete cytokines such as tumor necrosis factor alpha (TNF-α) and interleukin-1β(IL-1β) which activate fibroblasts and induce the overproduction of extracellular matrix (ECM) proteins (2). In the murine model of bleomycin-induced pulmonary fibrosis, inflammasome activation and IL-1R/MyD88 signaling are critical aspects of the profibrotic activity of IL-1β (3). Transient expression of IL-1β alone in the rat lung has been shown to result in tissue damage and progressive fibrosis (4). Transgenic overexpression of IL-1β in the murine pancreas also results in a fibrotic pancreatitis (5).

Numerous studies have implicated the type 2 cytokines, IL-4, IL-5 and IL-13, in driving progressive fibrosis (6). IL-4 can directly induce mouse and human fibroblasts to synthesize ECM proteins (6-8). IL-13 secreted by Th2 cells mediates fibrotic remodeling in a TGF-β dependent or independent manner in experimental lung fibrosis, and may contribute to the pathogenesis of idiopathic pulmonary fibrosis, systemic sclerosis, dermatitis induced skin fibrosis and liver fibrosis induced by persistent infections (9-14). M2 macrophages that have been triggered by IL-4 and IL-13 can induce other cells to produce IL-4, IL-10, IL-13 and TGF-β and thus contribute to fibrosis. Macrophages involved in wound healing also secrete large amounts of TGF-β (15). Although fibrosis is generally linked to what is presumably the uncontrolled activity of Th2 cells, M2 macrophages and fibroblasts, the prominent role in fibrosis of IL-β, a cytokine typically made by M1 macrophages, indicates that such Th2 biased models can be applicable to a subset of fibrotic diseases and that a presumed Th2 basis for a number of fibrotic diseases may sometimes represent an oversimplification of a complex and poorly understood pathogenic processes.

IgG4-related disease (IgG4-RD) is a chronic inflammatory syndrome whose pathogenesis is poorly understood. This disease can affect virtually every organ system of the body and is characterized by tumefactive lesions, storiform fibrosis, obliterative phlebitis and the presence in diseased tissues of IgG4 secreting plasma cells. The analysis of circulating Th1 and Th2 cells has led to conflicting results in IgG4-RD subjects. One study reported a Th1 skew in peripheral blood T cells in autoimmune pancreatitis while other studies in IgG4-RD patients with lacrimal gland enlargement showed an increase in Th2 phenotype cells in the peripheral blood (16-19).

As described herein, we have recently reported that atopic manifestations are seen in about 40% of IgG4-RD subjects, a proportion that is within the range seen in the population at large (20). We also performed studies on GATA-3 expressing circulating Th2 cells in IgG4-RD subjects. Circulating GATA-3^{±} IL-4, IL-5 and IL-13 secreting Th2 cells were only found in IgG4-RD subjects with concomitant atopy (21). Since expansions of circulating Th2 cells were not observed in IgG4-RD subjects without atopy, we undertook an unbiased next-generation sequencing approach to study the clonality of effector CD4⁺ T cells in patients with active untreated IgG4-RD, in an effort to identify the antigen-driven clonal expansion of effector CD4⁺ T cells in subjects with this disease. We report herein that CD4⁺ T cells with a cytotoxic T lymphoid phenotype are clonally expanded in IgG4-RD subjects. These unusual CD4⁺ T cells can synthesize and secrete IL-1β following TCR or TLR triggering and apart from being expanded in the blood are also found within diseased tissue sites. Their numbers decline concomitant with a clinical response to rituximab therapy, indicating a contributory role for these IL-1β secreting CD4⁺ cytotoxic T cells in the pathogenesis of this systemic fibrotic disease.

### Effector Memory T Cells are Expanded in IgG4-RD

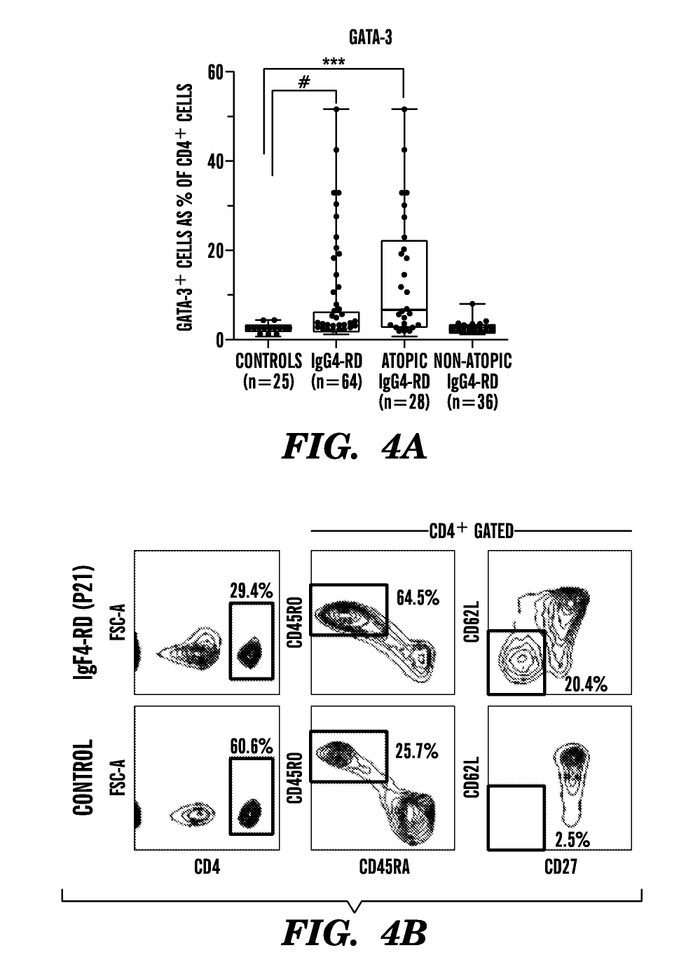

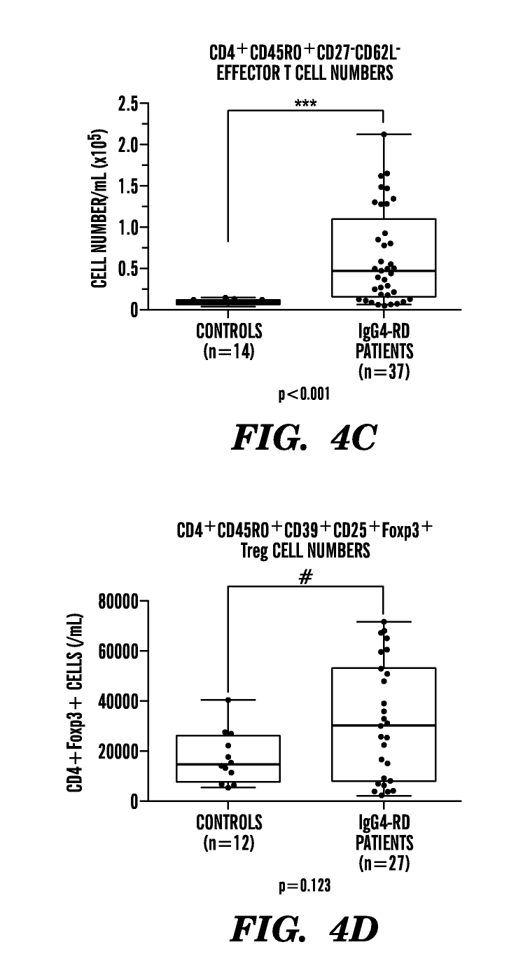

T cells are abundant in the lymphocytic infiltrate in IgG4-RD tissue lesions (22). In addition, both the immunoglobulin class switch and the high degree of somatic hypermutation that characterize the humoral immune response in IgG4-RD are T-cell dependent events (23). Based on circumstantial evidence from peripheral blood and disease lesions, Th2 cells have been implicated in the pathogenesis of IgG4-RD. We validated our previous findings (21) in a larger cohort of IgG4-RD patients and found a mild but statistically insignificant increase in CD4⁺ T cells with a Th2 phenotype as determined by GATA3 expression (FIG. 4A) as compared with non-atopic controls. As reported earlier, subsets of patients with concomitant atopy were observed to have a strong correlation with the presence of GATA3⁺ memory Th2 cells in circulation (21; FIG. 4A) while non-atopic IgG4-RD subjects did not exhibit an increase in cells of this T cell subset.

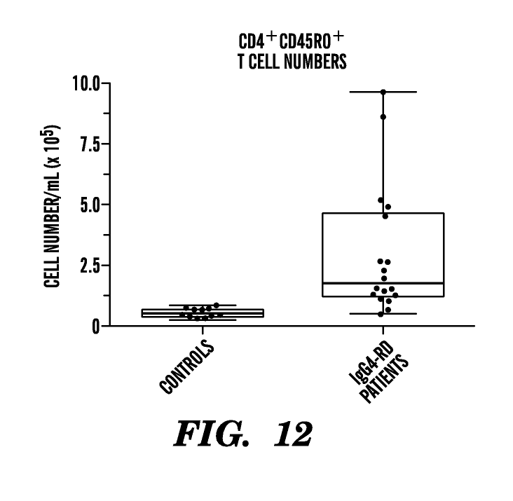

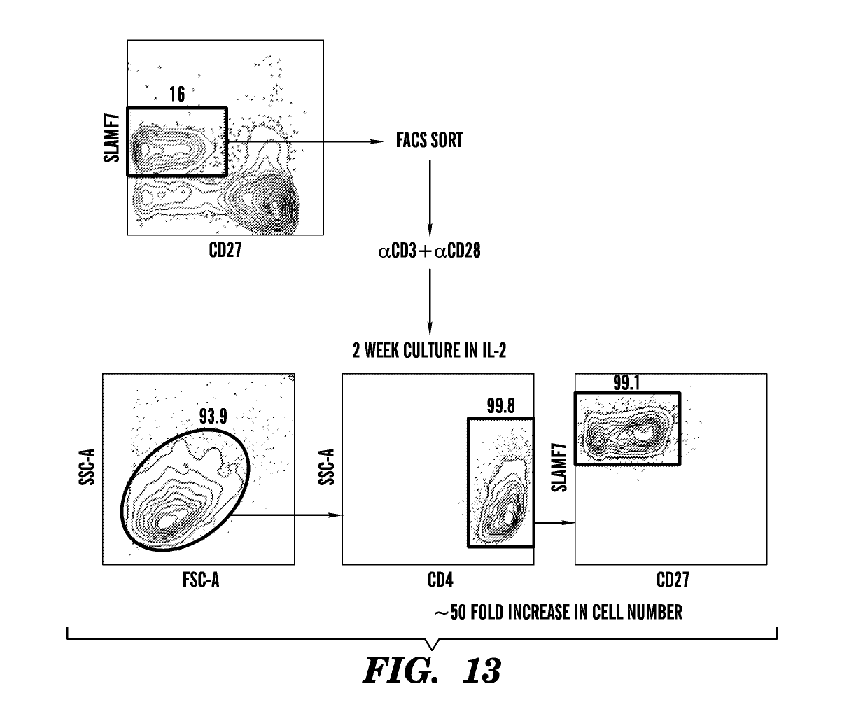

We investigated IgG4-RD subjects with active disease for the presence of CD4⁺ T-cell populations that have an effector/memory phenotype presumably resulting from persistent antigenic stimulation (24, 25). The numbers and frequency of CD4⁺CD45RO⁺ antigen experienced cells were increased in IgG4-RD patients compared to healthy controls (FIG. 4A and FIG. 12). Within this population, we observed an expansion of the CD27^{lo}CD62L^{lo} T cell subset in the majority of IgG4-RD subjects analyzed (FIG. 4B and 4C). CD4⁺CD45RO⁺CD27^{lo}CD62L^{lo} cells represent CD4⁺ T effector/memory (T_{EM}) cells, which arise from persistent exposure to potential auto-antigens (24). In addition, a marginal increase was observed in regulatory T cells in the effector pool that were identified as CD4⁺CD45RO⁺CD39⁺CD25⁺ Foxp3⁺ cells in PBMCs from IgG4-RD subjects; this difference was not statistically significant when compared to controls (FIG. 4D) (16, 18, 27). Given the selective increase of CD4⁺ T_{EM} cells in IgG4-RD patients, we decided to characterize these cells further and investigate their abundance and role in IgG4-RD.

### Expanded _{TEM} Cells Have a Highly Restricted TCRβ Repertoire

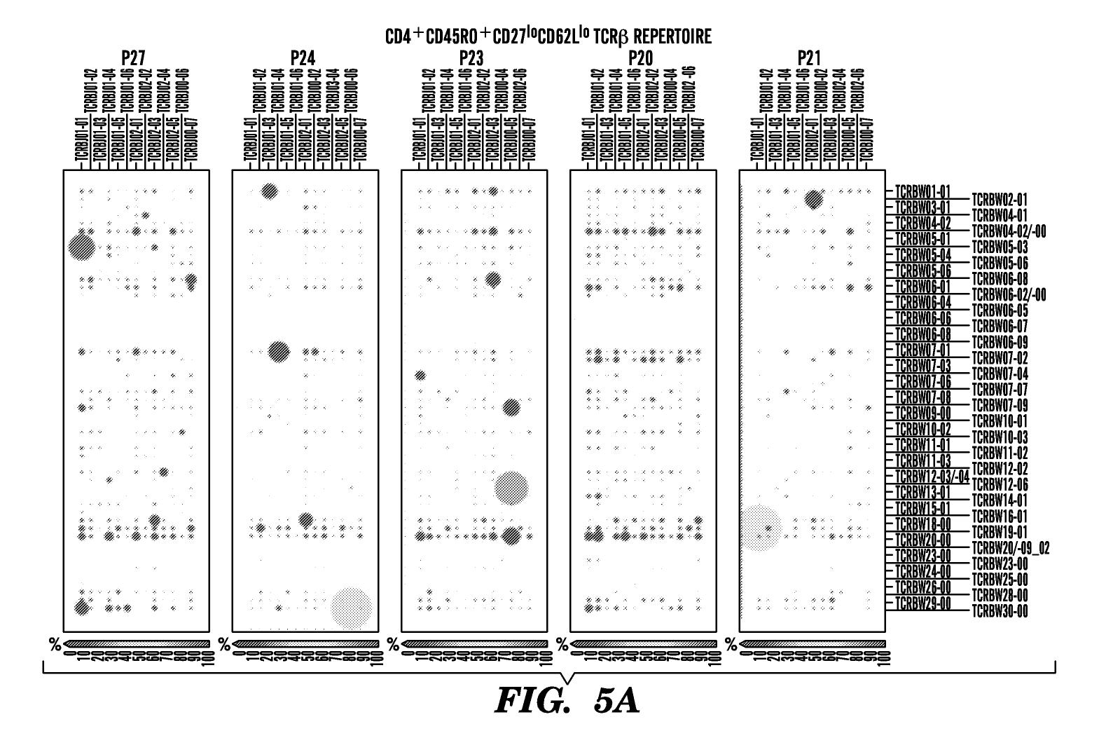

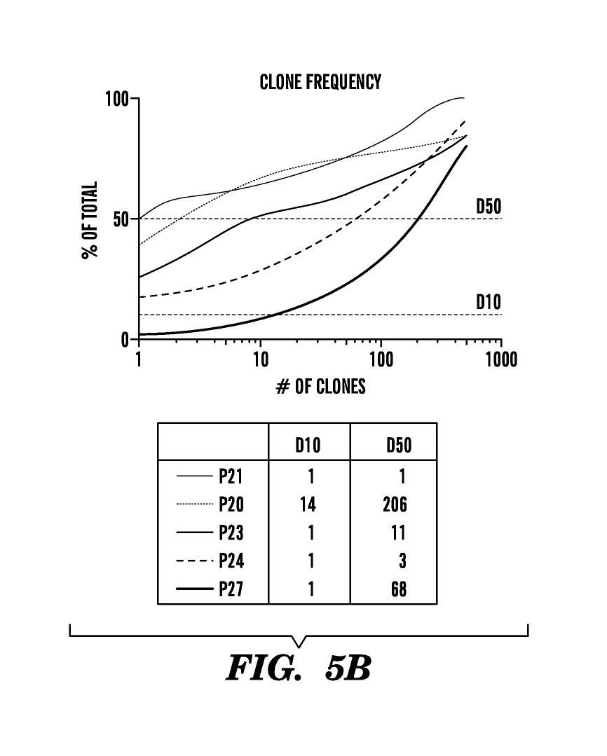

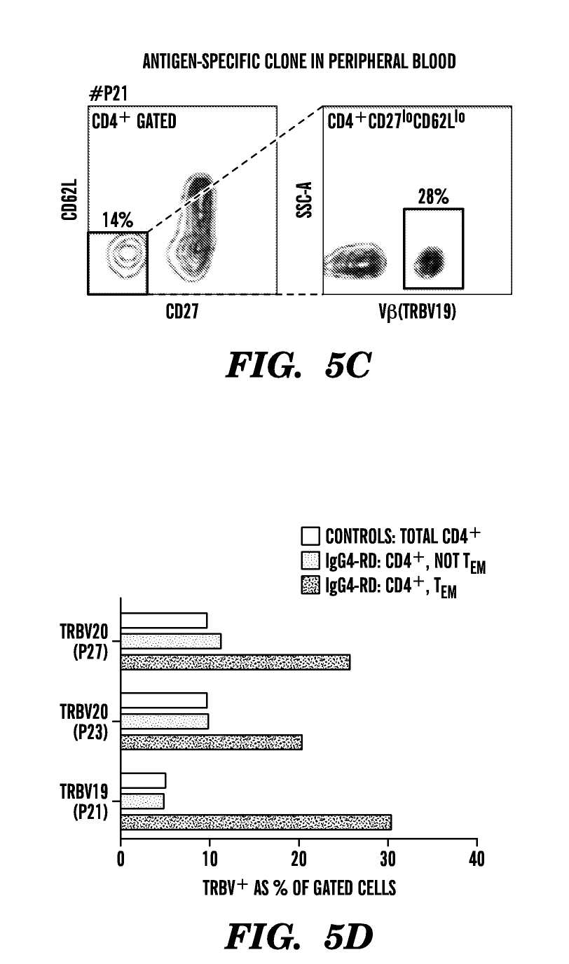

We wished to determine whether there were clonal expansions of CD4⁺T_{EM} cells by using next-generation sequencing of rearranged TCRβ chain genes in order to identify potentially pathogenic T cell subsets that were expanded in response to a potential antigen (self or foreign). We accordingly analyzed the TCRβ chain gene repertoire of T_{EM} cells from five untreated subjects with active IgG4-RD. We observed oligoclonal expansions of these T_{EM} cells, typically with a single dominant clone representing 3% to 50% of all the in-frame V-D-J rearrangements detected (FIGS. 5A and 5B). Several minor clones were also seen in each patient (FIG. 5A). The Vβ-Jβ gene segment usage of the most expanded clones was not identical across subjects and there were no clones with shared CDR3 sequences across individuals. The TCR Vβ gene usage in a subset of expanded T_{EM} clones as determined by Next-Generation Sequencing was validated using TCR Vβ-specific antibodies in all 3 subjects for whom Vβ-specific antibodies could be obtained (FIGS. 5C and 5D).

### Disease-Related T_{EM} Cells are CD4⁺ T-bet⁺ CD28^{lo} SLAMF7⁺ GZMB⁺ PRF1⁺ Cytotoxic T Lymphocytes Seen in the Blood and Also in Fibrotic Lesions

We sought to determine whether there was any similarity between the expanded effector CD4⁺ T_{IM} cells found in different subjects with IgG4-RD with very different clinical presentations. A focused gene expression analysis of 458 immune-related genes using a NANOSTRING codeset was performed on the expanded _{TEM} cells from 4 IgG4-RD patients, all with active disease manifesting in different organs. Very similar gene expression changes were seen in all 4 samples (FIG. 6A). Surprisingly, IL-1β was expressed at high levels in these T cells and this was also the cytokine gene with the maximum fold-difference in expression when compared to CD4⁺CD45RO⁺ T memory cells from healthy controls. A number of genes expressed in cytotoxic T lymphocytes (perforin, granzyme B, 2B4, T-bet and IFN-γ) were also expressed at higher levels in these disease related T_{EM} cells as was cell surface SLAMF7 and the chemokines, CCL4 and CCL5. The expression of many of these molecules was validated at the protein level using flow cytometry and a large proportion of the expanded CD4⁺ T_{EM} cells in disease subjects co-expressed SLAMF7, perforin, granzyme B and T-bet (FIG. 6B). The above markers were also found to be co-expressed on a single expanded clone identified using the corresponding TCR Vβ-specific antibody, suggesting that the expanded clones represent a uniformly differentiated sub-population that can be tracked by the expression of SLAMF7 and T-bet (FIG. 6C).

The clonally expanded CD4⁺CTLs maintained their phenotype in vitro in presence of anti-CD3 stimulation and recombinant human IL-2 (20 ng/mL) (FIG. 12). These cells bear a striking resemblance to cytotoxic CD4⁺ T lymphocytes reported in mice in the context of chronic antigenic stimulation (28). The development of these cells in mice is associated with the gain of Eomes and Runx3 and the loss of ThPO expression (28). Like the murine CD4⁺ CTLs, CD4⁺SLAMF7⁺ T cells from IgG4-RD patients exhibited decreased levels of ThPOK and increased expression of Runx3 when compared to CD4⁺CD45RA⁺ naïve cells and CD4⁺CD45RO⁺ memory cells from healthy controls (FIG. 6D). These cells also express surface CD8α as has been reported in mice (FIG. 6E) (28). Upon in vitro stimulation with anti-CD3, these cells undergo degranulation as inferred from the surface expression of CD107α (FIG. 6F) and also exhibit cytotoxic activity against co-cultured allogeneic EBV-transformed B cell targets (FIG. 6G and FIG. 14). Of note, none of the genes characteristic of Th2 cells were over-expressed in the expanded CD4⁺SLAMF7⁺ T cells. These expanded CD4⁺SLAMF7⁺ T cells therefore have polarized in an unusual manner and share some features of cytotoxic T cells and some of myeloid cells.

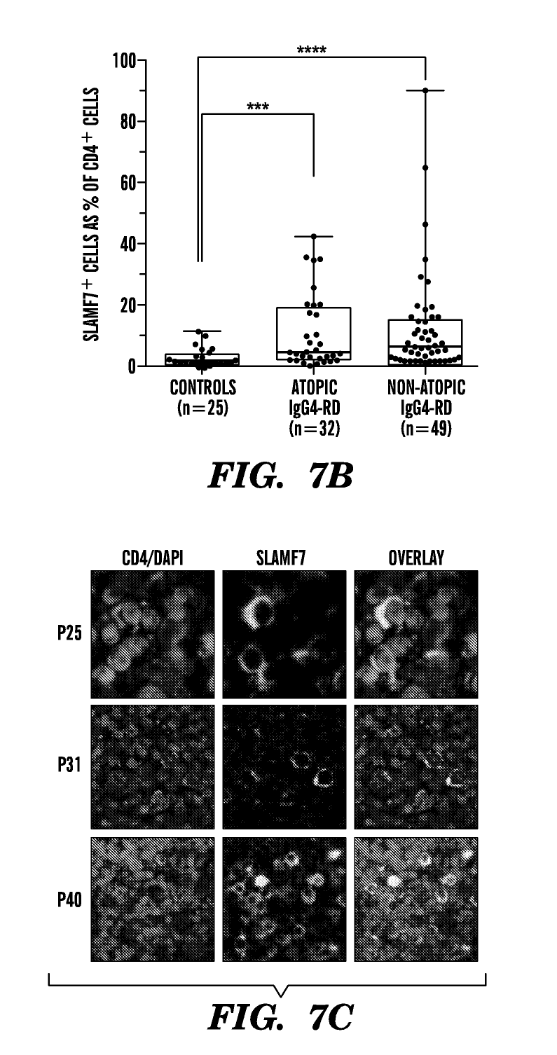

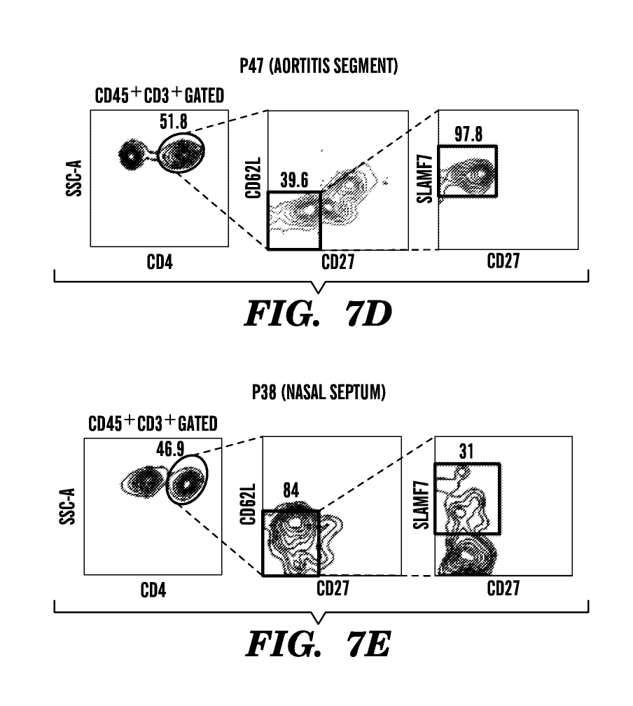

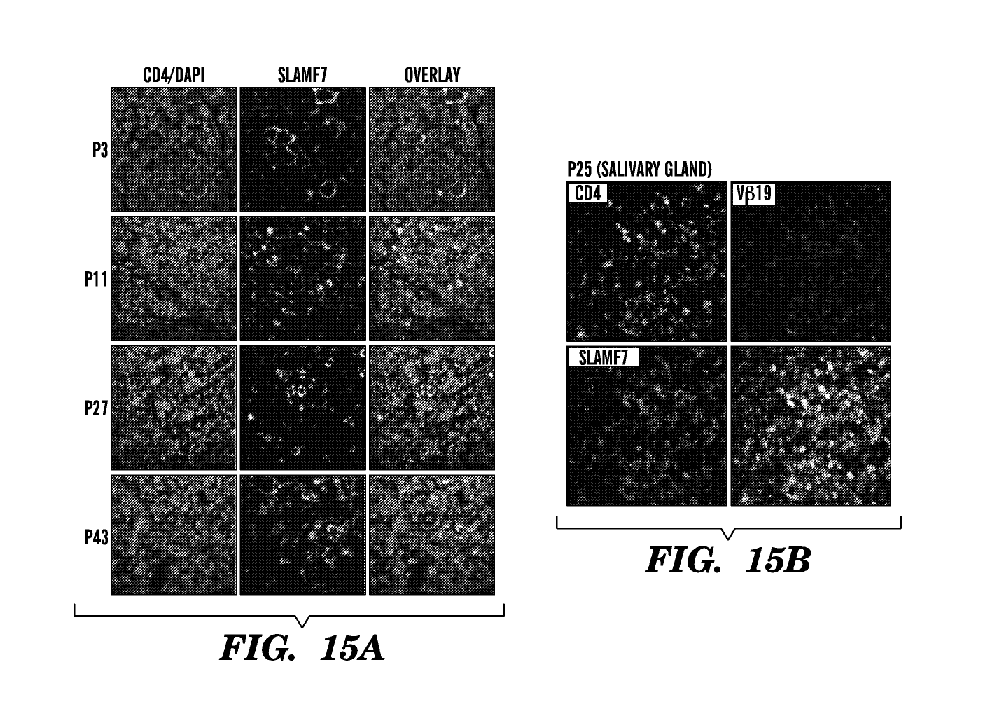

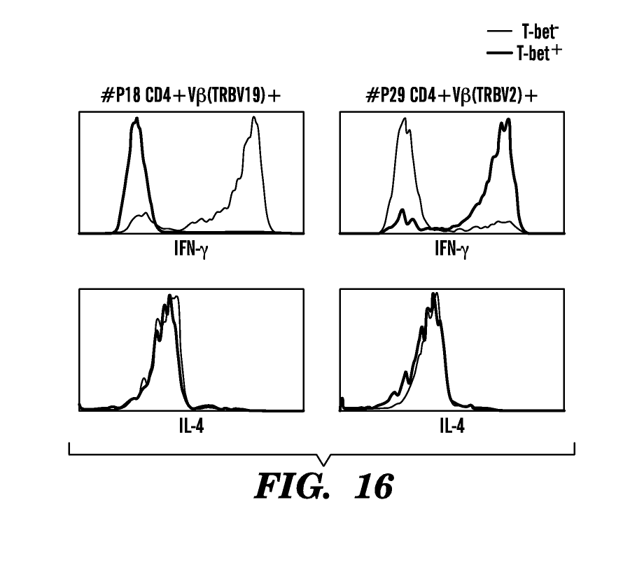

CD4⁺SLAMF7⁺ CTLs cells were elevated in the peripheral blood of 88 IgG4-RD subjects studied compared to healthy controls (p<0.01) (FIG. 7A). Unlike circulating GATA-3⁺ Th2 memory cells, CD4⁺CTLs are equally distributed among patients with or without atopy (FIG. 7B). This underlines the importance of these cells in IgG4-RD pathogenesis. Multi-color immunofluorescence staining of the affected organs of 6 IgG4 RD subjects showed tissue infiltration of CD4⁺SLAMF7⁺ CTLs (FIG. 7C and FIG. 15A). Indeed, the expanded Vβ19⁺ T-cell clone that was dominant in the circulation in this patient (FIG. 5A) was also abundant in the inflamed tissue (FIG. 15B). A segment of diseased aorta obtained at autopsy from a previously described patient (P47), who succumbed to IgG4-related aortitis showed the presence of CD4⁺SLAMF7⁺ cells in the vessel wall by flow cytometric analysis (FIG. 7D) (29). Similarly, CD4⁺SLAMF7⁺ cells were also detected in the biopsy of the involved nasal septum from a patient (P38) with IgG4-related midline destructive lesion (FIG. 7E) (30). However no accumulation of Th2 phenotype cells was observed the tissue samples analyzed. These data indicate that the expanded CD4⁺CTL cells represent an unusual CD4⁺ T cell subset that may be directly involved in driving the pathology of the affected tissues.

### Th2 Phenotype Cells are not Central to the Pathogenesis of IgG4-RD

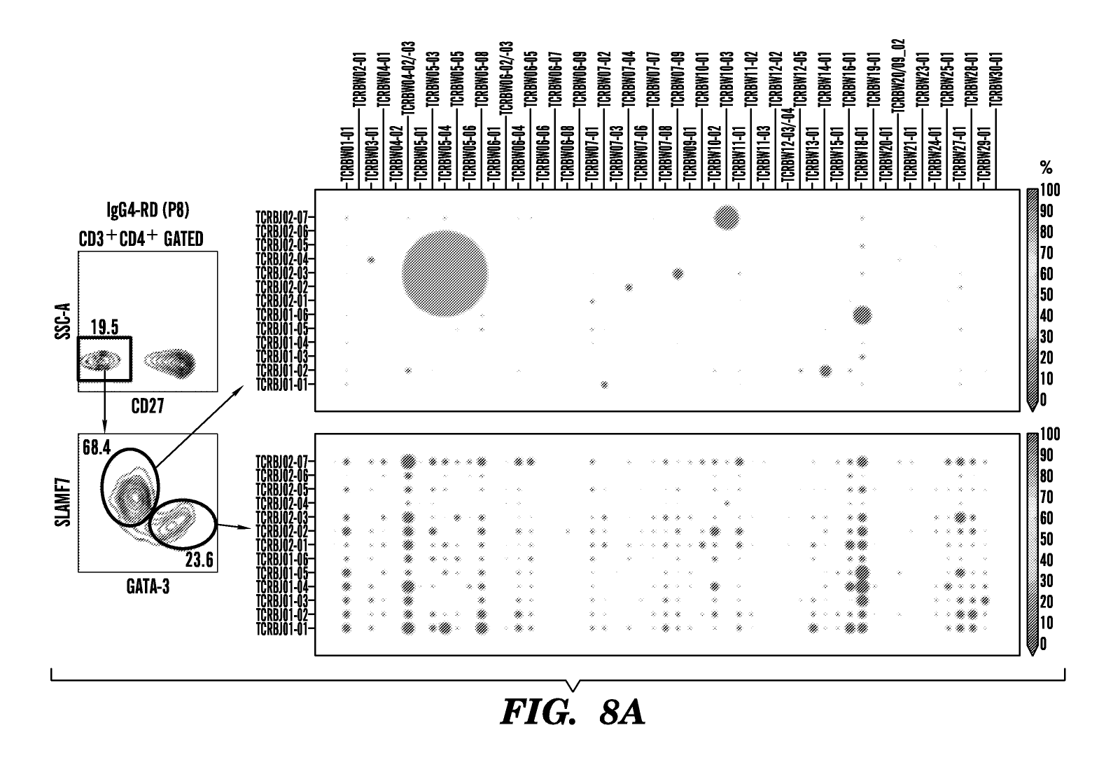

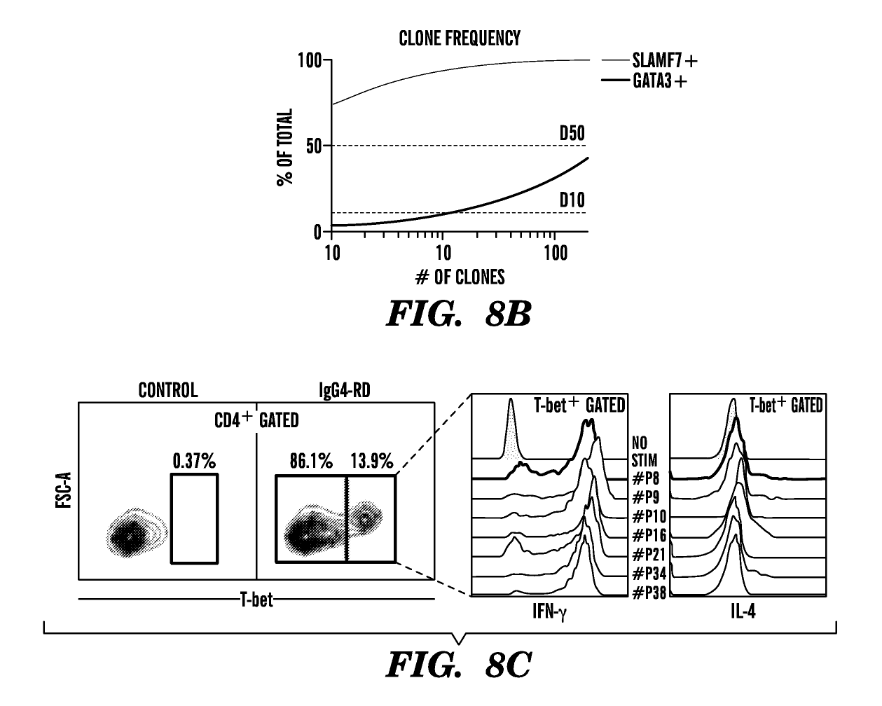

Concurrent expansions of circulating Th2 cells are observed in only a subset of IgG4-RD patients with atopy (21). We analyzed the TCRβ repertoire of CD4 effector subsets in an IgG4-RD subject with both expanded GATA-3⁺ memory Th2 cells and CD4⁺SLAMF7⁺ CTLs by next-generation sequencing. CD4⁺SLAMF7⁺ CTLs were highly oligoclonal with one expanded clone representing ˜80% of the productive sequencing reads (FIGS. 8A and 8B). In contrast, the most expanded clones represented <2% of the sequencing reads in the GATA-3⁺Th2 cells (FIGS. 8A and 8B). These data strongly indicate that CD4⁺SLAMF7⁺ CTLs have expanded in response to a specific causal antigen, possibly an auto-antigen, and thus have a direct role in disease pathogenesis, while the repertoire of Th2 phenotype cells may reflect the accumulated T cell memory against a wide range of environmental allergens.

In order to understand the functional behavior of the disease-related CD4⁺CTLs, peripheral blood mononuclear cells were briefly re-stimulated with PMA and ionomycin to mimic TCR signaling, followed by intracellular staining for T-bet, IFN-γ and IL-4. Since T-bet is a lineage-determining transcription factor that is exclusively expressed in all the clonally expanded CD4⁺CTLs in IgG4-RD subjects (FIGS. 6B and 6C), we identified re-stimulated CD4⁺CTLs among total CD4⁺ lymphocytes using T-bet staining.

We did not identify any T-bet⁺ cells lacking the expression of perforin or granzyme B, that could represent bona fide Th1 cells, in these patients (FIGS. 6B and 6C). Re-stimulated CD4⁺ CTLs cells from all IgG4-RD patients made large amounts of IFN-y but no IL-4 (FIG. 8C). Clonally expanded CD4⁺ CTLs, identified using specific TCR Vβ antibody staining, consistently expressed IFN-y but not IL-4 upon re-stimulation (FIG. 16).

As mentioned above, Th2 cell expansions are not observed in the blood in most patients with IgG4-RD without concurrent atopic disease (21). The data presented herein indicate that Th2 cells in the blood of an IgG4-RD patients with concomitant atopy appear not be clonally expanded. Tissue sites that have been analyzed, all in patients without concurrent atopy, contain CD4⁺ CTLs that correspond to clonal expansions seen in the blood but Th2 cells were not seen at these sites. These data indicate that Th2 cells may not be of pathogenic relevance in IgG4-RD while CD4⁺ CTLs appear, by all criteria examined, to be linked to the pathogenesis of this disease.

### Clonally Expanded Disease-Related CD4⁺SLAMF7⁺CD4 CTLs Make IL1-β Upon Restimulation

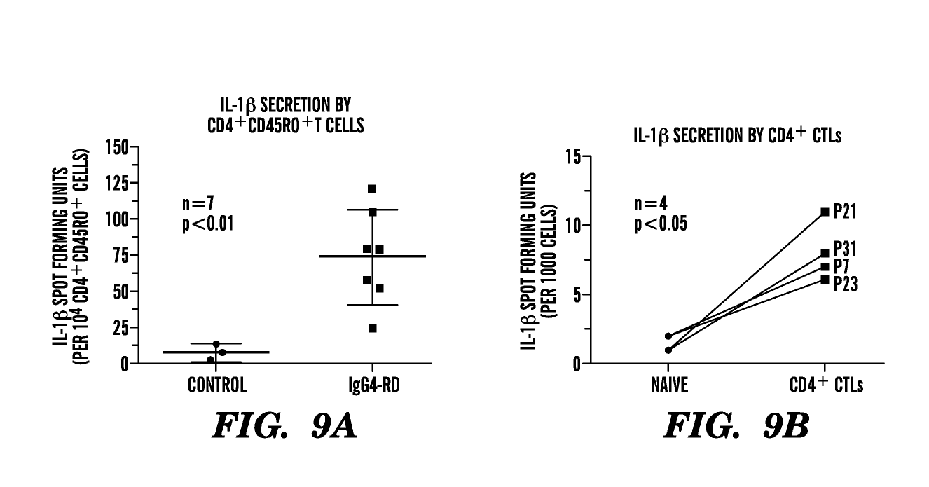

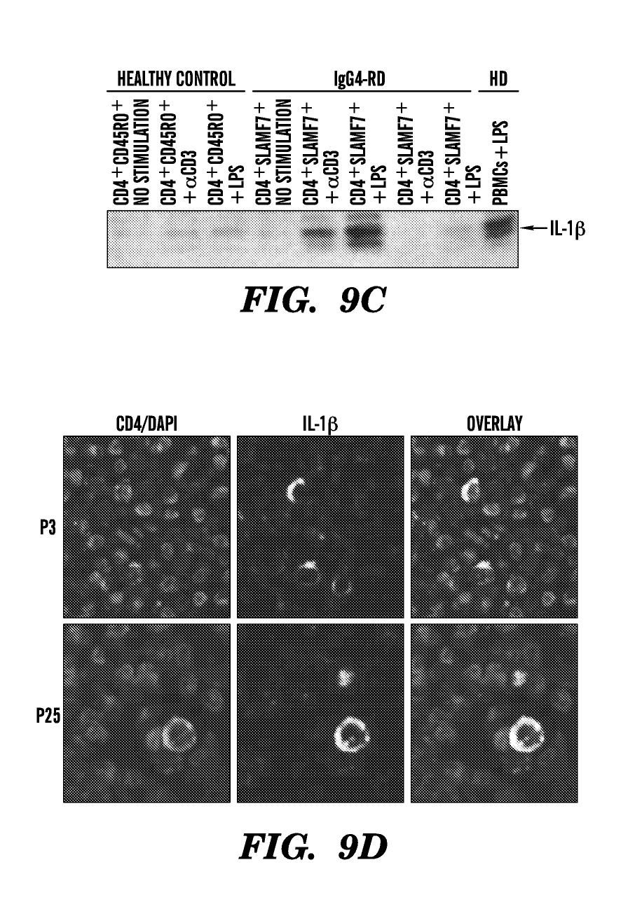

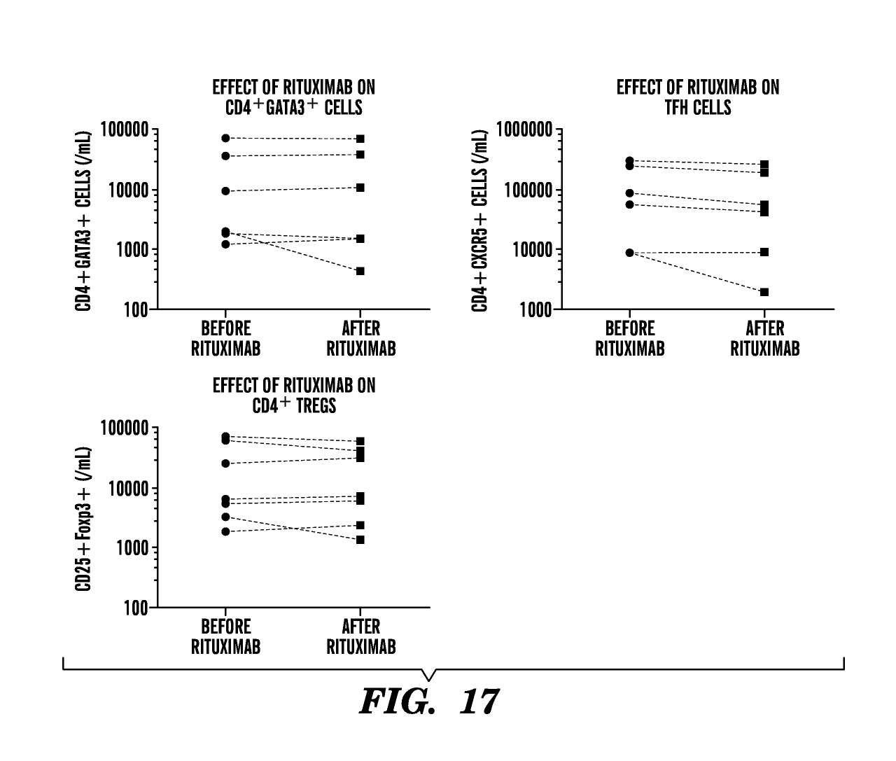

IL-1β was the most prominent cytokine at the mRNA level in the expanded TEM population and it was also analyzed at the protein level along with other cytokines more typically seen in effector T cells. Upon in vitro restimulation with PMA and ionomycin, IL-1β producing cells were enriched in flow-sorted CD4⁺CD45RO⁺ T cells from IgG4-RD subjects as detected by ELISPOT analysis compared to CD4⁺CD45RO⁺ T cells from healthy controls (FIG. 9A). Flow-sorted CD4⁺ CTLs cells contained more IL-1β producers compared to naïve CD4⁺CD45RA⁺ T cells from four IgG4-RD subjects tested (FIG. 9B). These cells could be expanded in vitro and maintained in IL-2 with intermittent TCR stimulation. The expanded CD4⁺ CTLs retained their SLAMF7 expression and cytotoxic markers and were found to secrete the processed form (17 KDa) of IL-1β upon restimulation with either anti-CD3 or LPS as determined by Western blot analysis of culture supernatants (FIG. 9C). The amount of IL-1β secreted was comparable to the amount secreted by myeloid cells exposed to LPS. Multi-color immunofluorescence staining of the affected organs of 4 IgG4-RD subjects showed high expression of IL-1β in a large proportion of CD4⁺ cells (FIG. 9D and FIG. 17).

### Rituximab Depletes Circulating CD4⁺SLAMF7⁺ CTLs in IgG4-RD Subjects

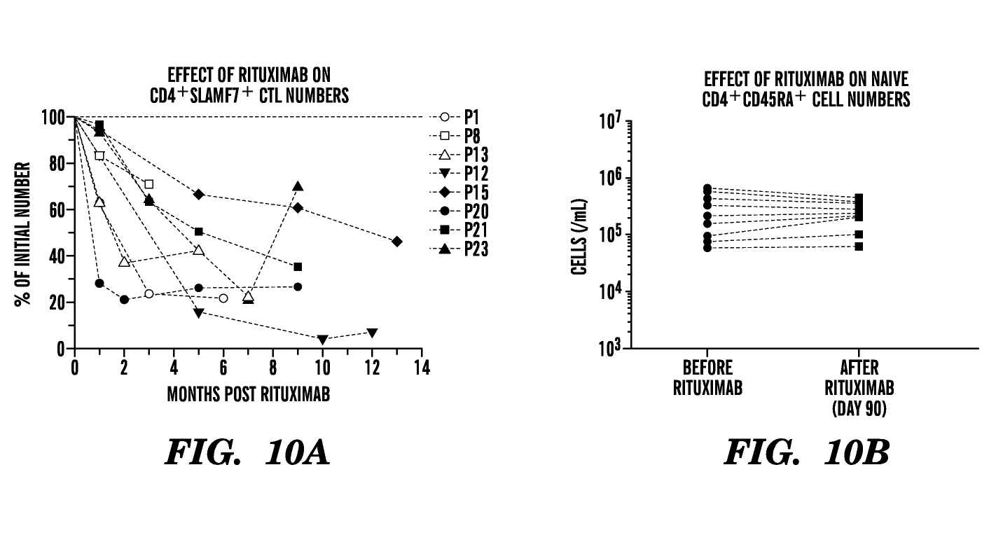

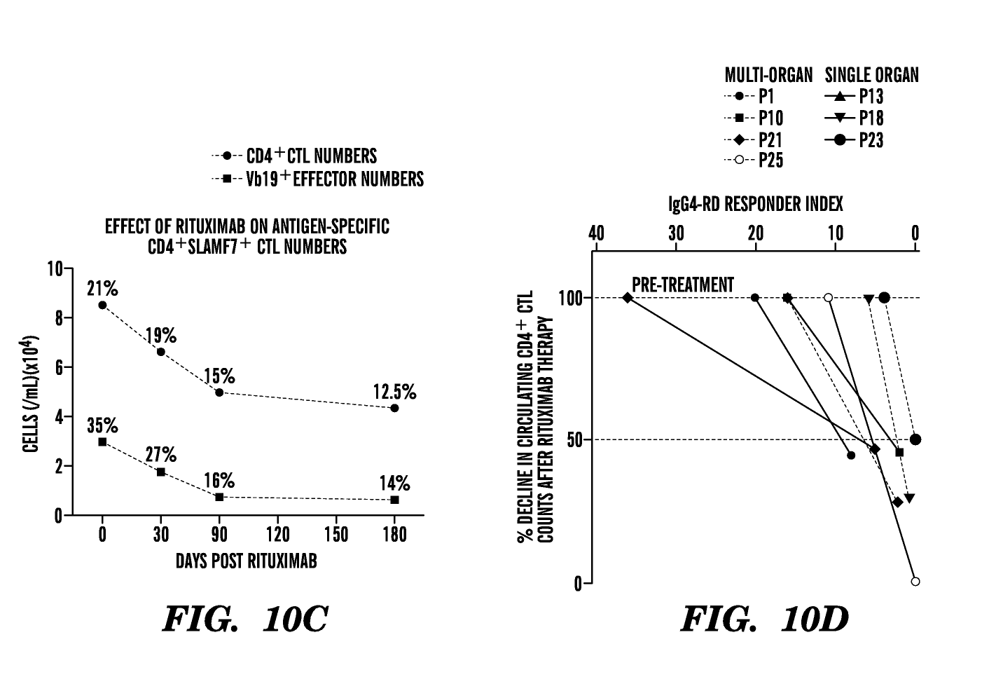

Therapeutic B-cell depletion with rituximab (an anti-CD20 monoclonal antibody) generally results in clinical improvement in IgG4-RD (31, 32). CD19⁺ B cells decline dramatically after rituximab therapy, followed by a clinically apparent reduction in disease activity as measured using the IgG4-RD Responder Index (33). Interestingly, a significant decrease in the percentages and numbers of CD4⁺SLAMF7⁺ CTLs was also observed up to 12 months after rituximab treatment while the number of naïve CD4⁺CD45RA⁺ T cells remained stable (FIGS. 10A & 10B). In one serially monitored subject, for whom a TCR Vβ-specific antibody was available, rituximab treatment induced a decline in the Vβ19⁺ CTL clone as well as of total CD4⁺SLAMF7⁺ CTLs (FIG. 10C). A positive clinical response to rituximab therapy was accompanied by a greater than 50% reduction in circulating CD4⁺SLAMF7⁺ CTL numbers (FIG. 10B). Rituximab, however, had minimal or no impact on the frequency and number of CD4⁺GATA3⁺ Th2 phenotype cells, CD4⁺CXCR5⁺ memory _{TFH} cells, or CD4⁺CD25⁺Foxp3⁺T regulatory cells in peripheral blood (FIG. 18). These data provide strong evidence for a pathogenic role for IL-1β secreting CD4⁺ CTLs in IgG4-RD.

### Circulating CD4⁺SLAMF7⁺ CTL Counts are Increased in IgG4-RD as Well as Other Immune-Mediated Fibrotic Conditions

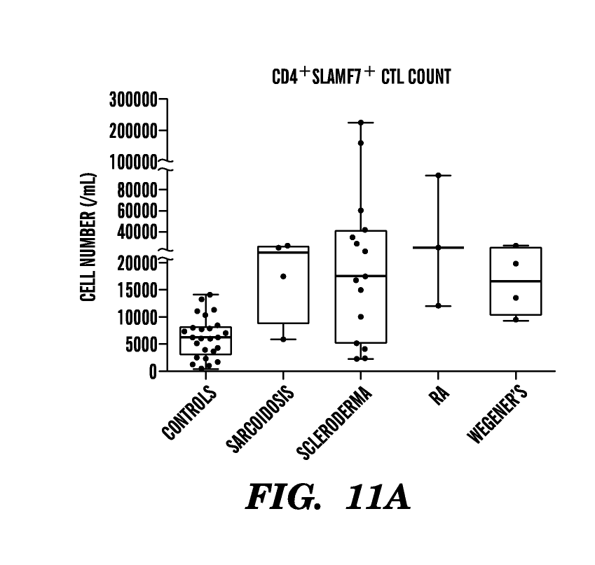

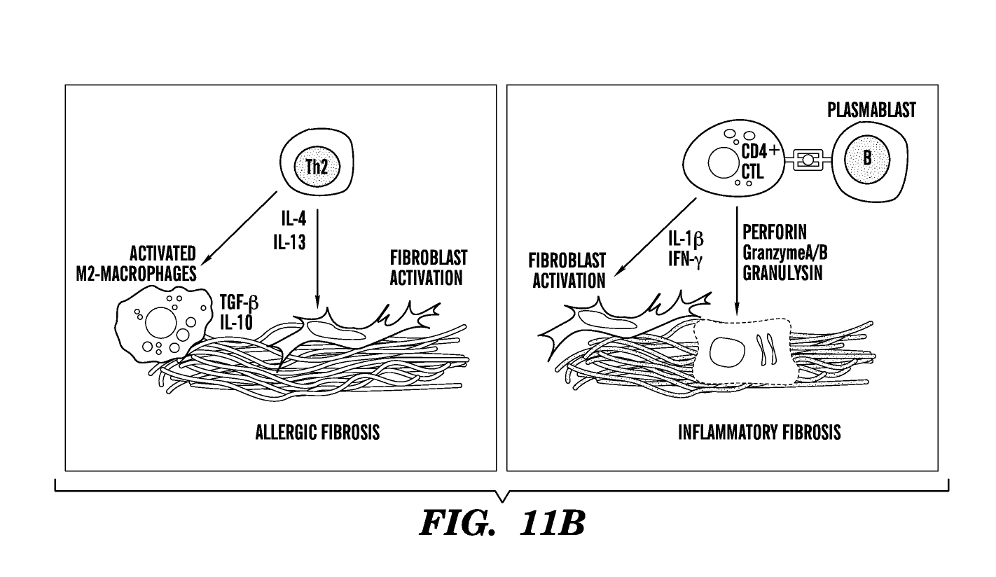

Since fibrosis is a commonly observed complication in many T-cell mediated autoimmune diseases, we tested if CD4⁺ CTLs are elevated in the circulation in such patients relative to healthy controls. We examined patients with active disease with clinical diagnoses of rheumatoid arthritis, systemic sclerosis, granulomatosis with polyangiitis (formerly Wegener's granulomatosis), or sarcoidosis and found a significant elevation in the CD4⁺ CTL counts of these subjects compared to healthy controls (p<0.01) (FIG. 11A). These CD4⁺ CTLs exhibited the same constellation of markers as seen in IgG4-RD and expressed T-bet, SLAMF7, CD 11b and 2B4. Thus this subset is not unique to IgG4-RD and may be of pathogenic relevance in a broad range of human autoimmune diseases with fibrotic manifestations.

### Discussion

One of the challenges in human immunology is the difficulty in advancing from correlation to causation. However, clonal expansion established by next-generation sequencing of a specific type of lymphocyte with unique functional attributes, its localization at sites of disease, and its decline upon successful therapy provide strong evidence for a causal role for IL-1β producing, CD4+ cytotoxic T cells in IgG4-related disease, and other fibrotic inflammatory disorders. Such an approach, determining which lymphocytes are clonally expanded and infiltrate tissue sites of disease, can serve as a template for implicating specific adaptive immune cells in disease processes.

The studies described herein demonstrate several novel and clinically relevant findings. Oligoclonal expansions of circulating CD4⁺SLAMF7⁺ CTLs are seen in IgG4-RD patients, especially during active disease. These T-cell clones are unusual in terms of their ability to make both IL-1β and IFN-γ, the former being a cytokine typically thought to be made by non-lymphoid cells (34). These expanded T cells were also found in affected tissues and our ability to correlate CD4⁺SLAMF7⁺ CTL expansions in IgG4 RD patients with the severity of their clinical presentations provides direct evidence for their role in disease pathogenesis. The production of large amounts of IL-1β by these T cells following brief re-stimulation, the expression of pre-formed cytotoxic mediators, and the strong correlation of this subset with disease activity indicates that the CD4⁺SLAMF7⁺ CTL population represents an unusual and not easily categorized subset of human CD4⁺ effector T cells that drives this disease, perhaps, without wishing to be bound or limited by theory, in collaboration with other T cells.

Fibrosis is an essential feature of the histopathology of IgG4-RD (22), but the etiology of the fibrosis in IgG4-RD has not been clear. IgG4 antibodies are generally considered to be non-inflammatory since they do not efficiently engage activating Fc receptors and complement and may be functionally monovalent in vivo (35). We have observed active disease in a number of subjects with relatively low levels of plasma IgG4 but a clear expansion of the CD4⁺SLAMF7⁺ CTL population. The participation, if at all, of IgG4 antibodies in disease pathogenesis is therefore unclear. It is possible that the IgG4 response seen in disease represents an exaggerated but ineffectual attempt to dampen inflammation.

As described herein, our observations show that clonally-expanded CD4⁺ CTLs that express granzyme B, perforin, IFN-y and IL-1β are prominent in this disease and can induce the fibrotic pathology seen in IgG4-RD. Transient expression of IL-1β alone in the rat lung has been shown to result in tissue damage and progressive fibrosis (4). Transgenic overexpression of IL-1β in the murine pancreas results in a fibrotic pancreatitis (5). IL-1R1/MyD88 signaling and the inflammasome are essential drivers of the fibrotic process in the widely studied murine model of pulmonary fibrosis induced by bleomycin (3). IFN-γ has been shown to contribute to fibrosis in a murine thyroiditis model (36). Indeed IL-1β induced fibrosis in mice has been shown to be dependent on IL-17A and IFN-γ. These CD4⁺ CTLs observed in IgG4-RD represent an abundant source of both IL-1β and IFN-γ in the tissue lesions of IgG4-RD, thereby having the ability to drive fibrosis. DNA sequence based analyses of HLA class II alleles has been performed on 24 subjects with IgG4 RD.

In addition to our studies on IgG4-RD, studies on smaller numbers of subjects with other inflammatory fibrotic diseases indicate that CD4⁺Tbet⁺SLAMF7⁺CD11b⁺2B4⁺ CTLs contribute to the pathogenesis of a range of fibrotic inflammatory diseases. CD4⁺ CD28^{lo} cells, which have been shown to express cytotoxic mediators and IFN-γ in some studies, have previously been identified in idiopathic pulmonary fibrosis, severe rheumatoid arthritis, multiple sclerosis and granulomatosis with polyangiitis (formerly Wegener's granulomatosis) (16, 37-39) and a small proportion of healthy elderly subjects. The CD4⁺SLAMF7⁺ CTLs that we have identified in IgG4-RD subjects, also express reduced levels of CD28 and may be related to these previously described CD4⁺CD28^{lo} cells. A survey of SLAM family protein expression in systemic lupus erythematosus revealed higher levels of SLAMF7 in some B and T cells, but the cell phenotypes were not characterized further (40).

Many types of fibrosis including murine models of schistosomiasis and bleomycin-induced pulmonary fibrosis have been linked to Th2 cells and Th2 cytokines (6). Although Th2 cells have been previously implicated in IgG4-RD in some studies (16-18), we have recently demonstrated that Th2 cell expansions are highly correlated with allergic history of the patients. We have not seen a consistent expansion of Th2 effector cells in active IgG4-RD. Instead, we find that IgG4-RD subjects with chronic allergies in addition to fibrotic tumescent lesions have an expansion of both Th2 cells as well as IL-1β producing CD4⁺ CTLs. These CD4+ CTL's actually synthesize IL-1β in affected fibrotic tissues, indicating that fibrotic disease mechanisms that have been elucidated downstream of IL-1β are of importance in IgG4-RD.

Analysis of a subject with expansions of both CD4⁺ CTL as well as Th2 memory cells revealed that although the CD4⁺ CTL exhibit large oligoclonal expansions, the GATA-3⁺Th2 memory cells have a highly diverse TCR β repertoire. The oligoclonal expansion of CD4⁺ CTLs but not Th2 phenotype cells within the same individual strongly implies that CD4⁺ CTL expansions are driven by disease related antigens, perhaps auto-antigen(s), and are likely to contribute to pathogenesis. In contrast Th2 memory cells are more likely to accumulate in response to environmental allergens and may not play a role in IgG4-RD disease pathogenesis. The absence of expansions of Th2 cells in the peripheral blood in the majority of IgG4-RD subjects, the lack of clonal expansions of Th2 cells in subjects with concomitant allergy although CD4⁺ CTLs are clonally expanded, and the inability to identify bona fide Th2 cells in fibrotic lesions (that however do contain clonally expanded CD4⁺ CTLs) taken together indicate that Th2 cells, which may be crucial in the pathogenesis of a subset of fibrotic diseases, are not of pathogenic relevance in IgG4-RD.

We consider it likely that IL-4 producing follicular helper T cells (T_{FH} cells) may be relevant to the IgG4 class switch event that occurs in subjects with IgG4-RD and that such T_{FH} cells may be discovered in the blood, in draining lymph nodes and occasionally in tertiary lymphoid organs in this disease. We suspect that previous reports using immunohistochemical detection to identify occasional IL-4 producing cells in tissues (16-18) may well have identified T_{FH} cells linked to the IgG4 class switch that may have play no role in the fibrotic process. It is worth noting that the molecular basis for the induction of IL-4 gene expression in Th2 cells and _{TFH} cells is distinct (41, 42). Our studies indicate that the inflammatory fibrosis driven by CD4⁺ CTLs in IgG4-RD is distinct from the fibrosis seen in Th2-mediated allergies and helminthic infestations (FIG. 12B). We have recently demonstrated marked expansions of IgG4⁺ plasmablasts in IgG4-RD subjects with active disease (43). Since these plasmablasts express high levels of MHC class II molecules, are depleted by rituximab, and are also present in disease lesions, it is possible that they play an important role in the reactivation of the CD4⁺CTLs and induce them to either make inflammatory cytokines including IL-1β, or to kill parenchymal cells by a non-apoptotic mechanism in this inflammatory milieu. The loss of parenchymal cells, especially in subjects with telomerase mutations is an established pathogenic process in a subset of subjects with idiopathic pulmonary fibrosis but it is unclear what role if any parenchymal cell death plays in the induction of remodeling and fibrosis observed in IgG4-RD (44).

Subjects lacking prominent allergic symptoms have prominent CD4⁺ CTLs but lack Th2 effectors expressing GATA-3 and IL-4. However we do not discount the possibility that some IL-4 producing cells and IL-1β secreting CD4⁺ CTLs might collaborate in the process of pathogenesis. These CD4⁺ CTLs do not produce known inhibitory cytokines such as IL-10 or TGF-β, although these cytokines have been implicated in fibrosis as well. Interestingly, these CD4⁺ CTLs secreted IL1-β in response to a LPS, suggesting that the effector function of these cells may be modulated by innate microbial signals in the diseased tissues. From our data, we cannot exclude the possibility that the CD4⁺ CTLs originate from further differentiation of Th1 cells.

The selective decline in CD4⁺ CTLs including the expanded clones after rituximab-mediated B-cell depletion has broad relevance. These observations imply that in humans, as in rodents, B cells play an important role in the maintenance of effector/memory CD4 T cells, including disease-associated, “rogue” T-cell clones (45, 46). These findings help explain why rituximab is effective in diseases such as relapsing-remitting multiple sclerosis in which end-organ damage is primarily mediated by autoreactive T cells. B cells that have the unique ability to bind specific autoantigens can act as potent antigen-presenting cells at low antigen concentrations (47), and can provide efficient access to T-cell epitopes. Thus, in T-cell mediated autoimmune disorders that are responsive to rituximab therapy, the effector/memory CD4 T cells response is maintained by B cells. Alternatively, pathogenic T cells can be dependent upon B cell derived growth factors (45). These possibilities are not mutually exclusive.

From a therapeutic standpoint, depleting SLAMF7-expressing cells as well as neutralizing IL1-β can represent novel, rational strategies in a range of immune-mediated conditions associated with severe tissue damage and fibrosis. A few biologics targeting SLAMF7 or IL1-β are already in the market or under advanced stages of drug development. A humanized monoclonal antibody directed against the human SLAMF7, elotuzumab, has shown promise in patients with advanced multiple myeloma and is being pursued in phase III clinical trials (48) Anakinra, a non-glycosylated recombinant form of the naturally occurring IL-1 receptor antagonist which blocks inflammasome dependent IL1-β signaling has been successfully used in type 2 diabetes, asbestosis, and other conditions (49). Canakinumab is a moncolonal antibody that binds to and antagonizes IL-1β and is being studied in a number of clinical trials (50).

In summary, our studies described herein indicate that CD4⁺ CTLs with a unique hitherto undescribed phenotype clonally expand in the circulation and tissue sites and can mediate the pathological changes seen in IgG4-RD. These cells make a unique combination of cytokines some of which have been shown to contribute to fibrosis in animal models, and the numbers of these cells correlate well with clinical disease activity. Furthermore, therapeutic improvement in IgG4-RD mediated by B cell depletion is linked to a specific reduction of these CD4⁺ CTLs and not of naive T cells, regulatory T cells or memory T follicular helper cells. Examining untreated active disease has allowed the identification and characterization of clonally expanded effector T cells linked to disease and to the observation of their attenuation by rituximab.

### MATERIALS AND METHODS

### Patients

This study was approved by the institutional review board and informed, written consent was obtained from all subjects with IgG4-RD referred to or presenting at the rheumatology clinic of the Massachusetts General Hospital. Samples from 90 patients with IgG4-RD were chosen for this study. The IgG4-RD patients were compared with 25 healthy controls (age 32-70 years). Twenty-three of these patients with active disease were treated with two 1000 mg doses of rituximab, 15 days apart. 15 ml of peripheral blood was collected at initial presentation and each subsequent clinical visit. Twelve of the rituximab-treated patients were longitudinally followed for 9-15 months at the rheumatology clinic. Peripheral blood was collected in EDTA or ACD tubes (BD VACUTAINER) and transported to the laboratory for cell isolation on the same day.

### Isolation of mononuclear cells

Mononuclear cells were isolated from peripheral blood of IgG4-RD subjects and healthy controls by FICOLL-PAQUE PLUS (GE HEALTHCARE) density-gradient centrifugation following the manufacturer's protocol. To facilitate subsequent analysis of cells in batches, PBMCs were resuspended in fetal bovine serum containing 10% dimethyl sulfoxide and cryopreserved in vapor phase liquid nitrogen.

### Immunofluorescence

Anti-human TCRVβ19-PE (clone ELL1.4, BECKMAN-COULTER), anti human CD4-biotin and streptavidin APC (LIFE TECHNOLOGIES), mouse anti-human CD319 and goat anti-mouse Alexa fluor 488 were used for immunofluorescence detection of clonally-expanded T cells in a deparaffinized section of a submandibular salivary gland biopsy using established protocols.

### Flow Cytometric Analysis

Fluorescence labeling for flow cytometry was performed by incubating cells in staining buffer (BIOLEGEND) containing optimized concentrations of fluorochrome conjugated antibodies. Except where indicated, all antibodies were procured from BIOLEGEND. The following monoclonal antibodies were used in this study: anti-human CD19-Pacific Blue (clone HIB19), anti-human CD27-APC (clone O323), anti-human CD38-FITC (clone HIT2), anti-human IgG4 (clone 6025, SOUTHERN BIOTECH), anti human CD4-PECy7 (clone OKT4), anti-human CD8α-PE, anti-human CXCR5-PE (clone J252D4), CD39 (Clone A1), anti-human CD45RA-PE (clone HI100), anti-human CD45RO-APC (clone UCHL1), anti-human CD62L-FITC (clone DREG56), anti-human CD244-biotin (clone C1.7, EBIOSCIENCE), anti-human CD319 (SLAMF7)-PE (clone 162.1), anti-human CD11b-APC Cy7 (clone ICRF44), anti-human CD28-PerCP Cy5.5 (clone CD28.2), anti-human TCR V1323 FITC (clone aHUT7), anti-human TCR V1319 PE (clone ELL1.4, BECKMAN-COULTER), anti-human TCR V132-PE (clone MPB2D5, BECKMAN-COULTER). A table of concordance between the TCR Vβ gene nomenclatures and IMGT gene names, which is available on the IMGT website on the world wide web, was used to verify that the appropriate Vβ-specific antibody clones were selected to detect clonally-expanded T-cell clones identified by next-generation sequencing (51). For intracellular staining of transcription factors (T-bet, GATA-3 and Foxp3) as well as cytolytic molecules (granzyme B and perforin) cells were fixed and permeabilized with the Foxp3-staining kit (EBIOSCIENCE) according to manufacturer's guidelines. Cells were then stained in permeabilization buffer with anti-human T-bet PECy7 (clone 4B10, EBIOSCIENCE), antihuman GATA-3 PECy7 (clone L50-823, BD Biosciences), anti-human Foxp3 (clone 206D, BIOLEGEND), anti-human Perforin PE (clone B-D48) and anti-human granzyme B FITC (clone GB11). For the degranulation assay, CD4⁺SLAMF7⁺ CTLs were stimulated with 3 μg/mL anti-human CD3 (OKT3) for 4 hours and surface staining for anti-human CD107a (BIOLEGEND) was performed followed by permeabilization and intra-cellular staining for Granzyme B as discussed above.

EBV-transformed B cell lines from an IgG4-RD patient (P46) were used as targets in the allogeneic CTL assay. 5×10⁴ EBV-transformed B cells were co-cultured for 12 hours with CD4⁺ CTLs from two patients at different ratios in presence or absence of anti-CD3. Cells were harvested and surface stained for anti-human CD4 as described above followed by staining with Annexin V-APC in Annexin V binding buffer (15 minutes at room temperature). DAPI was added to the cells at a final concentration of 1 ug/ml. Target cells were gated as CD3-negative and percentage of apoptotic/dead cells were estimated by Annexin V⁺/DAPI⁺ gates.

### Intracellular Staining for Cytokines in Restimulated T Cells

For detecting intracellular levels of cytokines, mononuclear cells were stimulated with 100 ng/mL of phorbol-myristoyl acetate (SIGMA-ALDRICH) and 100 ng/mL of ionomycin (Invitrogen) in the presence of Brefeldin A (SIGMA-ALDRICH) for 4 hrs at 37° C. They were subsequently labeled with the LIVE/DEAD* fixable violet viability dye (1NVITROGEN) in phosphate-buffered saline for 20 minutes and stained for cell surface markers. Cells were then fixed/permeabilized and stained with anti-human IFN-γ APC (clone 4S.B3), anti-human IL-4 Alexa FLUOR® 488 (clone 8D4-8) for 45 minutes on ice. Cells were washed two times with permeabilization buffer and once with PBS and acquired/analyzed on a BD LSR II (BD BIOSCIENCES). The fcs files were analyzed using FLOWJO software (version 9.6.3, TREESTAR).

Cell sorting:

To preserve cell viability mononuclear cells were stained with relevant cell surface markers in DMEM (GIBCO) with ITS+ Universal Culture Supplement (BD BIOSCIENCES). For batch sorts, antibody-stained cells were resuspended at ˜20 million/mL in HBSS (GIBCO) plus 10 mM glucose and sorted on a BD FACSARIA II (BD BIOSCIENCES). Sorted cells were collected in 5 mL tubes containing 1 mL collection medium (DMEM with 30% FBS) and re-analyzed on the sorter to ensure >99% purity in defined gates.

Next-generation sequencing analysis of TCR Vβ repertoire:

Next-generation sequencing analysis of the TCR V13 repertoire was undertaken using the IMMUNOSEQ® platform at ADAPTIVE BIOTECHNOLOGIES INC. at the “survey” level of sequencing depth, that is designed to target an output of 200,000 assembled output sequences after preprocessing filters (52, 53). Briefly, genomic DNA was isolated from flow-sorted CD4⁺ T cell subsets from individual IgG4-RD subjects. Sorted cell numbers ranged from ˜5,000 to ˜40,000, assuring at least 5-fold depth of sequencing. Genomic rearrangements of V-D-J gene segments at the TCRβ locus were amplified using multiplex PCR with forward and reverse primers specific for Vβ and Jβ gene segments respectively, and analyzed by paired-end Illumina sequencing. Use of barcoded primers allowed multiplexing of next-generation sequencing samples on the ILLUMINA HI-SEQ instrument. The sequences were assembled in silico, and V-D-J regions were reconstructed, following standard IMGT gene nomenclature for TCR Vβ gene segments (51). Non-productive rearrangements were excluded from the analysis.

Gene-expression analysis:

The NCOUNTER® human immunology panel (NANOSTRING TECHNOLOGIES), comprising ˜500 immunology-related genes was used to quantify the gene expression of effector/memory T cells in IgG4-RD. RNA was extracted from 10,000-50,000 flow-sorted T cells using the QIAGEN RNAEasy Micro kit according to manufacturer's protocol. Targets were reverse-transcribed and pre-amplified for 14 cycles using the standard multiplexed target enrichment (MTE) protocol (NANOSTRING TECHNOLOGIES) for 458 target genes, which has been previously validated to yield a linear response. The amplified products were hybridized in solution to color-coded NCOUNTER capture and reporter probes and captured on an NCOUNTER Cartridge for high-resolution digital scanning and analysis on the GEN2 Digital Analyzer at NANOSTRING TECHNOLOGIES. The raw gene expression data was normalized to the mean of the spiked-in internal positive control probes to correct for technical assay variation and subsequently normalized to the mean of 15 housekeeping genes included in the NCOUNTER codeset to correct for differences in sample input or variation in reverse-transcription/pre-amplification. Biological replicates were evaluated for consistency and differential expression analysis of gene expression was undertaken using the ComparativeMarkerSelection module in the GenePattern pipeline (version 3.5.0) (54, 55).

Quantitative real time PCR:

Total RNA was extracted from ˜10,000-40,000 cells using the RNAEASY PLUS MICRO kit (QIAGEN). cDNA was synthesized using a RT2 first-strand kit (QIAGEN) followed by quantitative real time PCR analysis (SYBR green; LIFE TECHNOLOGIES). GAPDH mRNA expression was used as the normalizing control. The primers used were:

ThPOK
(F: 5′-gtctgccacaagatcatcca-3′ (SEQ ID NO: 13,
R: 5′-tcgtagctgtgcaggaagc-3′ SEQ ID NO: 14),
Runx3
(F: 5′cagaagctggaggaccagac-3′ SEQ ID NO: 15,
R: 5′-gtcggagaatgggttcagtt-3′ SEQ ID NO: 16)
&
GAPDH
(F: 5′-atgttcgtcatgggtgtgaa-3′ SEQ ID NO: 17,
R: 5′-gtcttctgggtggcagtgat-3′ SEQ ID NO: 18)

ELISpot and western blotting for IL-1β:

Prior to coating, 96-well polyvinylidene difluoride (PVDF) membrane plates (MILLIPORE) were prewet with 50 ul 70% ethanol/well for 2 minutes and washed three times with 200 ul sterile filtered water. ELISpot assay for IL-1β was performed using human IL-1β ELISPOT READY-SET-GO KIT (EBIOSCIENCE) according to the manufacturer's recommendations. In brief, plates were coated overnight at 4° C. with the anti-human IL-1β antibody provided, followed by gentle washing with ELISpot wash buffer (1X PBS plus 0.05% Tween-20) and blocking with complete medium (RPMI plus 10% fetal bovine serum) for 2 hours at room temperature. 10000 sorted CD4⁺CD45RO⁺ cells from healthy controls and IgG4-RD patients were rested on the plate in complete medium for 4 hours post-sorting, and stimulated with PMA (100 ng/ml) and ionomycin (100 ng/ml) overnight at 37° C. Cells were decanted gently using wash buffer to detach any adherent cells and plates were washed three times with wash buffer followed by incubation with the recommended dilution of biotinylated detection antibody for 2 hours at room temperature. After two additional washes, plates were incubated with horse-radish peroxidase conjugated with streptavidin for 45 minutes at room temperature. The plates were washed extensively 4 times with wash buffer followed by two additional washes with PBS to remove any traces of tween-20. 100 μL of TMB substrate (MABTECH) was added and spots were allowed to develop for upto 20 minutes. Counting and visual analysis of the spots were done using a computer-operated CTL ELISpot reader and the fraction of IL-1β secreting cells was quantified as the number of spots detected per 10,000 cells applied to the well.

For Western blot analysis of secreted IL-1β, CD4⁺CD45RO⁺ cells from healthy donors, CD4⁺CD27⁻ gated SLAMF7⁺ and SLAMF7⁻ cells from an IgG4-RD subject were sorted and expanded in vitro in a U-bottomed 96-well plate (BD FALCON) with weekly anti-CD3 stimulation (BIOLEGEND; OKT3, 3 μg/ml) in media supplemented with 10 ng/mL of rhIL-2. 150,000 cells were re-stimulated with anti-human CD3 (BIOLEGEND; OKT3, 3μg/ml) or Lipopolysaccharide (LPS; SIGMA, 5 μg/ml) and the culture supernatants were harvested at 60 hrs. These supernatants were run on an SDS-PAGE gel and transferred onto Immobilon-P membranes. IL-113 was detected using a rabbit anti-human IL-113 antibody (BIOVISION) followed by goat anti-rabbit Ig-HRP (THERMO SCIENTIFIC) and developed using SuperSignal West Pico Chemiluminescent Substrate (BIORAD). LPS stimulated PBMCs were used as positive controls.

### REFERENCES

1. G. Wick et al., The immunology of fibrosis: innate and adaptive responses. Trends Immunol 31, 110 (Mar, 2010).

2. T. A. Wynn, T. R. Ramalingam, Mechanisms of fibrosis: therapeutic translation for fibrotic disease. Nat Med 18, 1028 (July, 2012).

3. P. Gasse et al., IL-1R1/MyD88 signaling and the inflammasome are essential in pulmonary inflammation and fibrosis in mice. J Clin Invest 117, 3786 (December, 2007).

4. M. Kolb, P. J. Margetts, D. C. Anthony, F. Pitossi, J. Gauldie, Transient expression of IL-1beta induces acute lung injury and chronic repair leading to pulmonary fibrosis. J Clin Invest 107, 1529 (June, 2001).

5. F. Marrache et al., Overexpression of interleukin-1beta in the murine pancreas results in chronic pancreatitis. Gastroenterology 135, 1277 (October, 2008).

6. T. A. Wynn, Fibrotic disease and the T(H)1/T(H)2 paradigm. Nat Rev Immunol 4, 583 (August, 2004).

7. C. Doucet et al., Interleukin (IL) 4 and IL-13 act on human lung fibroblasts. Implication in asthma. J Clin Invest 101, 2129 (May 15, 1998).

8. G. D. Sempowski, M. P. Beckmann, S. Derdak, R. P. Phipps, Subsets of murine lung fibroblasts express membrane-bound and soluble IL-4 receptors. Role of IL-4 in enhancing fibroblast proliferation and collagen synthesis. J Immunol 152, 3606 (Apr. 1, 1994).

9. P. Fuschiotti, Role of IL-13 in systemic sclerosis. Cytokine 56, 544 (Dec, 2011).

10. J. E. Kolodsick et al., Protection from fluorescein isothiocyanate-induced fibrosis in IL-13-deficient, but not IL-4-deficient, mice results from impaired collagen synthesis by fibroblasts. Journal of immunology 172, 4068 (Apr. 1, 2004).

11. C. G. Lee et al., Interleukin-13 induces tissue fibrosis by selectively stimulating and activating transforming growth factor beta(1). J Exp Med 194, 809 (Sep. 17, 2001).

12. L. A. Murray et al., Hyper-responsiveness of IPF/UIP fibroblasts: interplay between TGFbetal, IL-13 and CCL2. The international journal of biochemistry& cell biology 40, 2174 (2008).

13. M. H. Oh et al., IL-13 induces skin fibrosis in atopic dermatitis by thymic stromal lymphopoietin. Journal of immunology 186, 7232 (Jun. 15, 2011).

14. H. L. Weng et al., The etiology of liver damage imparts cytokines transforming growth factor beta1 or interleukin-13 as driving forces in fibrogenesis. Hepatology 50, 230 (July, 2009).

15. T. A. Wynn, L. Barron, Macrophages: master regulators of inflammation and fibrosis. Semin Liver Dis 30, 245 (August, 2010).

16. Tanaka et al., Th2 and regulatory immune reactions contribute to IgG4 production and the initiation of Mikulicz disease. Arthritis and rheumatism 64, 254 (January, 2012).

17. H. Kanari et al., Role of Th2 cells in IgG4-related lacrimal gland enlargement. Int Arch Allergy Immunol 152 Suppl 1, 47 (2010).

18. Y. Zen et al., Th2 and regulatory immune reactions are increased in immunoglobin G4-related sclerosing pancreatitis and cholangitis. Hepatology 45, 1538 (June, 2007).

19. K. Okazaki et al., Autoimmune-related pancreatitis is associated with autoantibodies and a Th1/Th2-type cellular immune response. Gastroenterology 118, 573 (March, 2000).

20. E. Della Tone et al., Prevalence of atopy, eosinophilia, and IgE elevation in IgG4-related disease. Allergy 69, 269 (February, 2014).

21. H. Mattoo, E. Della-Tone, V. S. Mahajan, J. H. Stone, S. Pillai, Circulating Th2 memory cells in IgG4-related disease are restricted to a defined subset of subjects with atopy. Allergy 69, 399 (March, 2014). (September, 2012).

22. L. J. Maillette de Buy Wenniger et al., Immunoglobulin G4+ clones identified by next-generation sequencing dominate the B cell receptor repertoire in immunoglobulin G4 associated cholangitis. Hepatology 57, 2390 (June, 2013).

23. R. De Jong et al., The CD27-subset of peripheral blood memory CD4+ lymphocytes contains functionally differentiated T lymphocytes that develop by persistent antigenic stimulation in vivo. Eur J Immunol 22, 993 (April, 1992).

24. F. Sallusto, D. Lenig, R. Forster, M. Lipp, A. Lanzavecchia, Two subsets of memory T lymphocytes with distinct homing potentials and effector functions. Nature 401, 708 (Oct. 14, 1999).

25. R. Morita et al., Human blood CXCR5(+)CD4(+) T cells are counterparts of T follicular cells and contain specific subsets that differentially support antibody secretion. Immunity 34, 108 (Jan. 28, 2011).

26. M. Koyabu et al., Analysis of regulatory T cells and IgG4-positive plasma cells among patients of IgG4-related sclerosing cholangitis and autoimmune liver diseases. J Gastroenterol 45, 732 (July, 2010).

27. D. Mucida et al., Transcriptional reprogramming of mature CD4(+) helper T cells generates distinct MHC class II-restricted cytotoxic T lymphocytes. Nat Immunol 14, 281 (March, 2013).

28. J. H. Stone et al., IgG4-related systemic disease and lymphoplasmacytic aortitis. Arthritis and rheumatism 60, 3139 (October, 2009).

29. E. Della-Torre et al., IgG4-related midline destructive lesion. Ann Rheum Dis, (Mar. 20, 2014).

30. Khosroshahi et al., Rituximab for the treatment of IgG4-related disease: lessons from 10 consecutive patients. Medicine (Baltimore) 91, 57 (January, 2012).

31. Khosroshahi, D. B. Bloch, V. Deshpande, J. H. Stone, Rituximab therapy leads to rapid decline of serum IgG4 levels and prompt clinical improvement in IgG4-related systemic disease. Arthritis and rheumatism 62, 1755 (June, 2010).

32. M. N. Carruthers, J. H. Stone, V. Deshpande, A. Khosroshahi, Development of an IgG4-RD Responder Index. Int J Rheumatol 2012, 259408 (2012).

33. A. Dinarello, Immunological and inflammatory functions of the interleukin-1 family. Annual review of immunology 27, 519 (2009).

34. R. C. Aalberse, S. O. Stapel, J. Schuurman, T. Rispens, Immunoglobulin G4: an odd antibody. Clin Exp Allergy 39, 469 (April, 2009).

35. K. Chen, Y. Wei, G. C. Sharp, H. Braley-Mullen, Balance of proliferation and cell death between thyrocytes and myofibroblasts regulates thyroid fibrosis in granulomatous experimental autoimmune thyroiditis (G-EAT). J Leukoc Biol 77, 166 (February, 2005).

36. S. R. Gilani et al., CD28 down-regulation on circulating CD4 T-cells is associated with poor prognoses of patients with idiopathic pulmonary fibrosis. PLoS One 5, e8959 (2010).

37. Komocsi et al., Peripheral blood and granuloma CD4(+)CD28(−) T cells are a major source of interferon-gamma and tumor necrosis factor-alpha in Wegener's granulomatosis. Am J Pathol 160, 1717 (May, 2002).

38. P. B. Martens, J. J. Goronzy, D. Schaid, C. M. Weyand, Expansion of unusual CD4+ T cells in severe rheumatoid arthritis. Arthritis and rheumatism 40, 1106 (June, 1997).

39. J. R. Kim, S. O. Mathew, R. K. Patel, R. M. Pertusi, P. A. Mathew, Altered expression of signalling lymphocyte activation molecule (SLAM) family receptors CS1 (CD319) and 2B4 (CD244) in patients with systemic lupus erythematosus. Clinical and experimental immunology 160, 348 (June, 2010).

40. Yusuf et al., Germinal center T follicular helper cell IL-4 production is dependent on signaling lymphocytic activation molecule receptor (CD150). Journal of immunology 185, 190 (Jul. 1, 2010).

41. V. Deshpande et al., Consensus statement on the pathology of IgG4-related disease. Mod Pathol 25, 1181.

42. S. Crotty, Follicular helper CD4 T cells (TFH). Annual review of immunology 29, 621 (2011).

43. H. Mattoo et al., De novo oligoclonal expansions of circulating plasmablasts in active and relapsing IgG4-related disease. J Allergy Clin Immunol, (provisionally accepted) (2014).

44. M. P. Steele, D. A. Schwartz, Molecular mechanisms in progressive idiopathic pulmonary fibrosis. Annu Rev Med 64, 265 (2013).

45. T. A. Barr et al., B cell depletion therapy ameliorates autoimmune disease through ablation of IL-6-producing B cells. J Exp Med 209, 1001 (May 7, 2012).

46. Crawford, M. Macleod, T. Schumacher, L. Corlett, D. Gray, Primary T cell expansion and differentiation in vivo requires antigen presentation by B cells. Journal of immunology 176, 3498 (Mar. 15, 2006).

47. L. Rock, B. Benacerraf, A. K. Abbas, Antigen presentation by hapten-specific B lymphocytes. I. Role of surface immunoglobulin receptors. J Exp Med 160, 1102 (Oct. 1, 1984).

48. A. Zonder et al., A phase 1, multicenter, open-label, dose escalation study of elotuzumab in patients with advanced multiple myeloma. Blood 120, 552 (Jul. 19, 2012).

49. P. Menu, J. E. Vince, The NLRP3 inflammasome in health and disease: the good, the bad and the ugly. Clinical and experimental immunology 166, 1 (October, 2011).

50. Chakraborty et al., Pharmacokinetic and pharmacodynamic properties of canakinumab, a human anti-interleukin-1beta monoclonal antibody. Clinical pharmacokinetics 51, el (Jun. 1, 2012).

51. P. Lefranc et al., IMGT unique numbering for immunoglobulin and T cell receptor variable domains and Ig superfamily V-like domains. Dev Comp Immunol 27, 55 (January, 2003).

52. K. Larimore, M. W. McCormick, H. S. Robins, P. D. Greenberg, Shaping of human germline IgH repertoires revealed by deep sequencing. Journal of immunology 189, 3221 (Sep. 15, 2012).

53. H. S. Robins et al., Comprehensive assessment of T-cell receptor beta-chain diversity in alphabeta T cells. Blood 114, 4099 (Nov. 5, 2009).

54. J. Gould, G. Getz, S. Monti, M. Reich, J. P. Mesirov, Comparative gene marker selection suite. Bioinformatics 22, 1924 (Aug. 1, 2006).

55. Reich et al., GenePattern 2.0. Nat Genet 38, 500 (May, 2006).

We claim:

1. A method for treating a subject having an immune disease or disorder, the method comprising: administering a therapeutically effective amount of an inhibitor that binds SLAMF7 to a subject having an immune disease or disorder, thereby treating the immune disease or disorder.

2. The method of claim 1, wherein the inhibitor that binds SLAMF7 comprises an antibody.

3. The method of claim 1, wherein the immune disease or disorder comprises an IgG4-RD spectrum disorder or a fibrotic disease.

4. The method of claim 1, further comprising a step of diagnosing the subject as having the immune disease or disorder.

5. A method for diagnosing an immune disease or disorder, the method comprising:
(a) measuring the amount of SLAMF7 in a biological sample obtained from a suspected of having an immune disease or disorder, and (b) comparing the amount of SLAMF7 with a reference value, and if the amount of SLAMF7 is increased relative to the reference value, identifying the subject as having the immune disease or disorder.

6. The method of claim 5, wherein the immune disease or disorder comprises an IgG4-RD spectrum disorder or a fibrotic disease.

7. The method of claim 5, wherein the step of measuring the amount of SLAMF7 comprises contacting the biological sample with an antibody specific for SLAMF7.

8. The method of claim 5, wherein the reference value is obtained from a subject or population of subjects lacking a detectable immune disease or disorder.

9. The method of claim 5, further comprising measuring at least one additional cytotoxic CD4+ T-cell marker.

10. The method of claim 9, wherein the at least one additional cytotoxic CD4+ T-cell marker is selected from the group consisting of: CD11b, 2B4, granzyme, perforin, and T-bet transcription factor.

11. An assay comprising:
(a) measuring the amount of SLAMF7 in a biological sample obtained from a subject having, or suspected of having, an immune disease or disorder, and
(b) comparing the amount of SLAMF7 with a reference value, and if the amount of SLAMF7 is increased relative to the reference value, identifying the subject as having, or at risk of developing, an immune disease or disorder.

12. The assay of claim 11, wherein the immune disease or disorder comprises an IgG4-RD spectrum disorder or a fibrotic disorder.

13. The assay of claim 11, wherein the step of measuring the amount of SLAMF7 comprises contacting the biological sample with an antibody specific for SLAMF7.

14. The assay of claim 11, wherein the reference value is obtained from a subject or population of subjects lacking a detectable immune disease or disorder.

15. The assay of claim 11, further comprising measuring at least one additional cytotoxic CD4+ T-cell marker.

16. The assay of claim 15, wherein the at least one additional cytotoxic CD4+ T-cell marker is selected from the group consisting of: CD11b, 2B4, granzyme, perforin, and T-bet transcription factor.
